(19) United States
(12) Patent Application Publication
(10) Pub. No.: US 2025/0027162 A1
(43) Pub. Date: Jan. 23, 2025

(54) METHODS FOR DETECTING DYSFUNCTIONAL NK CELLS IN LEUKEMIA PATIENTS

(71) Applicants: City of Hope, Duarte, CA (US); The Board of Trustees of the Leland Stanford Junior University, Stanford, CA (US)

(72) Inventors: Srividya SWAMINATHAN, Duarte, CA (US); Holden Maecker, Palo Alto, CA (US); Anil Kumar, Duarte, CA (US); Caroline Duault, Palo Alto, CA (US); Adeleh Taghi Khani, Duarte, CA (US)

(21) Appl. No.: 18/289,455

(22) PCT Filed: May 9, 2022
(86) PCT No.: PCT/US2022/028371

§ 371 Date: Nov. 3, 2023

Related U.S. Application Data
(60) Provisional application No. 63/186,020, filed on May 7, 2021.

(51) Int. Cl. C12Q 1/6886; G01N 33/569; G01N 33/574

(52) U.S. Cl. CPC C12Q 1/6886; G01N 33/56972; G01N 33/57426; C12Q 2600/158; G01N 2333/70596; G01N 2800/54

(57) ABSTRACT

Provided herein are, inter alia, methods for identifying dysfunctional natural killer (NK) cells in a subject with leukemia. Provided are methods for treating leukemia, including administering to the subject an effective amount of allogeneic NK cells.

### CROSS-REFERENCE TO RELATED APPLICATIONS

This application claims priority to U.S. Provisional Application No. 63/186,020, filed May 7, 2021, which is hereby incorporated by reference in its entirety and for all purposes.

### STATEMENT AS TO RIGHTS TO INVENTIONS MADE UNDER FEDERALLY SPONSORED RESEARCH AND DEVELOPMENT

This invention was made with government support under 1U24CA224309 and P30CA033572 awarded by the National Institutes of Health. The government has certain rights in the invention.

### BACKGROUND

B- and T-cell acute lymphoblastic leukemia (B/T-ALL) are aggressive and often recur after therapy. Despite treatment advances, because of resistance to existing therapies, the 5-year relative survival is only of 68.8%. Therapeutic resistance is often caused by suppression of anti-leukemia host immunity. In studying immune surveillance in transgenic ALL mouse models, it was found that specific subversion of anti-leukemia natural killer (NK) cell-mediated surveillance drives ALL development and recurrence. Because of the relatively poor survival in B/T-ALL, there is a need for improved methods to identify the causes of such failure and improved methods of treatment. Provided herein are solutions to these and other needs in the art.

### BRIEF SUMMARY

In an aspect is provided a method of identifying dysfunctional natural killer (NK) cells in a population of NK cells obtained from a subject having leukemia, wherein the method includes detecting in the population of NK cells: (a) an elevated expression level of CD56 relative to a standard control; (b) an elevated expression level of CD94 relative to the standard control; (c) an elevated ratio of CD11b⁻CD27⁻ NK cells to CD11b⁺CD27⁻ NK cells relative to the standard control; (d) the presence of CD69; (e) the presence of a cytokine; or (f) calcium (Ca²⁺) mobilization; thereby identifying the dysfunctional NK cells.

In another aspect is provided a method of treating leukemia in a subject in need thereof, including administering to the subject an effective amount of allogeneic NK cells, wherein 50% or more of a population of NK cells obtained from the subject are dysfunctional NK cells.

In another aspect a method of treating leukemia in a subject in need thereof is provided, the method including: a) identifying dysfunctional natural killer (NK) cells in a population of NK cells obtained from the subject; and b) administering to the subject an effective amount of allogeneic NK cells; wherein 50% or more of the population of NK cells are dysfunctional NK cells.

In an aspect a method of treating leukemia in a subject in need thereof is provided including administering an effective amount of an engineered NK cell, wherein 20% of more of a population of NK cells obtained from the subject are dysfunctional NK cells.

In an aspect is provided a method of determining a probability of survival or relapse in a subject having leukemia, including identifying dysfunctional natural killer (NK) cells in a population of NK cells obtained from the subject, wherein 50% or more of the population of NK cells are dysfunctional NK cells indicates that the subject has decreased probability of survival or increased probability of relapse relative to a subject wherein less than 50% of the population of NK cells are dysfunctional NK cells.

In another aspect a method of identifying a subject susceptible to leukemia relapse is provided, including identifying dysfunctional natural killer (NK) cells in a population of NK cells obtained from the subject, wherein 50% or more of the population of NK cells are dysfunctional NK cells indicates that the subject is susceptible to leukemia relapse.

In another aspect, a method of detecting a dysfunctional NK cell in a leukemia patient is provided, the method including: detecting the presence of CD56 or CD69, wherein the presence of CD56 or CD69 indicates that the NK cell is a dysfunctional NK cell. In embodiments, the method further comprises detecting CD94 in the NK cell or plurality of NK cells. In embodiments, the method further includes detecting an elevated frequency of CD11b−CD27− cells and a reduced frequency of CD11b+CD27− cells relative to a standard control in a plurality of NK cells.

In a further aspect, provided herein is a method of treating leukemia in a leukemia patient previously treated for leukemia, the method comprising administering an effective amount of allogeneic NK cells. In embodiments, the leukemia patient is a relapse patient.

### BRIEF DESCRIPTION OF THE DRAWINGS

FIGS. 1A-1D show NK frequency and cytotoxicity is reduced in high-risk B/T-ALL patients. (FIG. 1A) CyTOF analysis depicting frequencies of total CD56⁺ HLA-DR⁻ NK cells (after gating out CD14⁺ and/or CD33⁺ monocytes, then CD3⁺ T cells, and then CD19⁺ and/or CD20⁺ B cells referred to as ‘non-monocyte non-T non-B’ gate) in BMMC and PBMC of B/T-ALL patients (n=12 BMMC, n=8 PBMC) and healthy donors (n=12 BMMC, n=10 PBMC). Data shown as median±interquartile range. (FIG. 1B) Representative viSNE plots showing surface CD56 expression in the non-T-cell, non-B-cell, non-monocyte, HLA-DR⁻ population in BMMC and PBMC of healthy donors and B/T-ALL patients. The color scale represents the intensity of CD56 expression on each cell. (FIGS. 1C-1D) comparison of NK specific cytotoxicity of sorted CD3⁻CD56⁺ NK effector cells from PBMC of healthy donors (n=3) and B-ALL patients (n=3) after co-culture with either the commonly used K562 erythroleukemia NK-sensitive target for measuring NK function (FIG. 1C) or NK-sensitive MOLT-4 T-ALL target cells (FIG. 1D) for 5 hours at effector:target=10:1. Data are shown from 3 independent experiments, with each experiment conducted using the same number of NK cells sorted from 1 B-ALL patient and 1 healthy donor. Each experimental pair is connected by a line. Exact p-value was calculated using the paired t-test.

FIGS. 2A-2D show NK cells with less cytotoxic CD56^{bright} molecular signature are expanded in B/T-ALL. (FIG. 2A) Schematic depicting the steps to estimate the relative proportions of CD56^{bright} NK and CD56^{dim} NK cells in healthy donors (GSE13159, GSE65136), in B/T-ALL patients from MILE study (GSE13159), and in B-ALL patients from the COG P9906 clinical trial (GSE11877) using CIBERSORT. Using GSE21774 which contains the transcriptomic profile of CD56^{bright} and CD56^{dim} NK subsets as the reference file, Applicant deconvoluted the bulk transcriptomic profiles of healthy donors, MILE B/T-ALL, and COG P9906 B-ALL patients. (FIG. 2B) Comparison of CD56^{bright} NK and CD56^{dim} NK frequencies in BMMC (top panel) of healthy donors (blue, n=74), B-ALL MILE patients (olive, n=540), T-ALL MILE patients (green, n=170), and B-ALL COG P9906 patients (red, n=131); and in PBMC (bottom panel) of healthy donors (blue, n=20) and B-ALL MILE patients (olive, n=36), T-ALL MILE patients (green, n=4), and B-ALL COG P9906 patients (red, n=76). Graphs show median±interquartile range. (FIG. 2C) Frequencies of CD56^{bright}CD27⁺, CD56^{bright}CD27⁻, CD56^{dim}CD27⁺, CD56^{dim}CD27⁻ NK subsets defined by CyTOF analysis of BMMC of healthy donors (n=12) and B/T-ALL patients (n=12). (FIG. 2D) Frequencies of CD56^{bight}CD27⁺, CD56^{bright}CD27⁻, CD56^{dim}CD27⁺, CD56^{dim}CD27⁻ NK subsets defined by CyTOF analysis of PBMC of healthy donors (n=10) and B/T-ALL patients (n=8). Graphs show mean±SEM for each subset. Exact p-values were calculated using the Mann-Whitney test. ns=nonsignificant.

FIGS. 3A-3D show maturation of NK cells into cytotoxic effectors is perturbed in PBMC of B/T-ALL patients. Schematics which illustrate analysis of NK cells showing the percentages of (FIG. 3A) CD94⁻, CD56^{bright}CD94⁺, CD56^{dim}CD94^{high}, and CD56^{dim}CD94^{low} NK cells (FIG. 3B), KIR2DL1⁺ total NK, CD56^{bright}KIR2DL1⁺, and CD56^{dim}KIR2DL1⁺ NK cells (FIG. 3C), CD11b⁻CD27−, CD11b⁻CD27⁺, CD11b⁺CD27⁺, and CD11b⁺CD27− NK cells (FIG. 3D) in PBMCs of healthy donors (n=9) and B/T-ALL patients (n=9), DNAM-1⁺ total NK, CD56^{bight}DNAM-1⁺, CD56^{dim}DNAM-1⁺ and CD56^{dim}DNAM-1⁻ NK cells in PBMCs of healthy donors (n=6) and B/T-ALL patients (n=6). Graphs show median±interquartile range. Exact p-values were calculated using the Mann-Whitney test. ns=nonsignificant. Gates for each marker were set using fluorescence minus one (FMO) controls.

FIGS. 4A-4F show stimulated NK cells in B/T-ALL patients produce more cytokines than their healthy counterparts. CyTOF analysis of the frequencies of PMA+ionomycin-stimulated NK cell subsets expressing GM-CSF (FIG. 4A), TNF-α (FIG. 4B), and IFN-γ (FIG. 4C) in BMMC of healthy donors (n=12) and B/T-ALL patients (n=12); and GM-CSF (FIG. 4D), TNF-α (FIG. 4E), and IFN-γ (FIG. 4F) in PBMC of healthy donors (n=10) and B/T-ALL patients (n=8). Graphs show median±interquartile range. Exact p-values were calculated using Mann-Whitney test, ns=not significant.

FIGS. 5A-5G show Hyperactivated and exhausted NK cells accumulate in peripheral blood of ALL patients. (FIG. 5A) Flow cytometry analysis depicting frequencies of CD69⁺ total NK, CD56^{bight}CD69⁺ and CD56^{dim}CD69⁺ NK cells in PBMC of healthy donors (n=9) and B/T-ALL patients (n=9). (FIG. 5B) Comparison of calcium flux in ionomycin-stimulated CD3-CD56⁺-gated NK cells depicting area under the curve from 6 healthy donors and 5 B-ALL patients. (FIG. 5C) Flow cytometry analysis depicting frequencies of CD69⁺CD94⁺ NK cells in PBMC of healthy donors (n=9) and B/T-ALL patients (n=9). CyTOF analysis of the frequencies of cells expressing LAG-3 (FIG. 5D), KLRG1 (FIG. 5E), PD-L2 (FIG. 5F), and Siglec-7 (FIG. 5G) in total NK cells, CD56^{bright} and CD56^{dim} NK subsets in PBMC of healthy donors (n=4) and B/T-ALL patients (n=9). Data are shown as median±interquartile range. Exact p-values was calculated using Mann-Whitney test.

FIGS. 6A-6G show high frequencies of activated NK cells predict poor clinical prognosis in high-risk B-ALL. (FIG. 6A) Schematic depicting the steps to estimate the relative proportions of activated and resting NK cells in B-ALL patients from the COG P9906 clinical trial (GSE11877) using CIBERSORT. The LM22 signature matrix (GSE22886), which contains the transcriptomic profile of ‘resting NK’ and cytokine (IL-2, IL-15)-‘activated NK’, was used to deconvolute the bulk transcriptomic profiles of COG P9906 B-ALL patients. After excluding 4 patients who lacked NK cells, remaining patients were assigned to two cohorts as ‘Resting NK>Activated NK’ (n=104) and ‘Activated NK>Resting NK’ (n=99). (FIGS. 6B-6G) Comparison of relapse-free survival probabilities of COG P9906 B-ALL patients divided into Resting NK>Activated NK (n=104) and Activated NK>Resting NK (n=99) groups (FIG. 6B); Resting NK>Activated NK CNS⁺ (n=21) and Activated NK>Resting NK CNS⁺ (n=24) groups (FIG. 6C); Resting NK>Activated NK MRD⁺(n=31), Activated NK>Resting NK MRD⁺(n=36), Resting NK>Activated NK MRD⁻ (n=63) and Activated NK>Resting NK MRD⁻ (n=57) groups (FIG. 6D); 100% activated NK (n=85) and 100% resting NK (n=73) groups (FIG. 6E); 100% activated NK CNS⁺ (n=19) and 100% resting NK CNS⁺ (n=14) groups (FIG. 6F); 100% activated NK MRD⁺(n=30), 100% resting NK MRD⁺(n=20), 100% activated NK MRD⁻ (n=49) and 100% resting NK MRD⁻ (n=44) groups (FIG. 6G). Survival curves using the Kaplan-Meier method are displayed. Exact p-values have been computed using the log-rank test.

FIGS. 7A-7G show high frequency of cytokine-producing NK cells predicts increased severity and poor prognosis of ALL. (FIG. 7A) Comparison of transcript levels of GM-CSF (CSF2), TNF-β (LTA), TNF-C(LTB) and IFN-γ between COG P9906 B-ALL patients (GSE11877) assigned to two groups using CIBERSORT as 100% resting NK (n=73) or 100% activated NK (n=85). Data are shown as median±interquartile range. (FIG. 7B) CIBERSORT comparing estimated relative proportions of resting and activated NK cells within total NK cells between GM-CSF^{High} TNF^{High} IFN-γ^{High} (n=22) and GM-CSF^{Low} TNF^{Low} IFN-γ^{Low} (n=19) COG P9906 B-ALL patients (GSE11877). Patients were assigned to high and low groups based on the median expression of each transcript. The LM22 signature matrix (GSE22886), which contains the transcriptomic profile of ‘resting NK’ and cytokine (IL-2, IL-15)-‘activated NK’, was used to deconvolute the bulk transcriptomic profiles of COG P9906 B-ALL patients. Data are shown as median±interquartile range. (FIG. 7C) Stacked bar charts comparing the proportions of GM-CSF^{High} TNF^{High} IFN-γ^{High} (n=22) and GM-CSF^{Low} TNF^{Low} IFN-γ^{Low} (n=19) COG P9906 B-ALL patients (GSE11877) with WBC>100,000, relapse, CNS involvement (CNS⁺), testicular involvement in male patients (Testicular⁺), and positive minimal residual disease on day 29 (MRD⁺). (FIG. 7D) Comparison of relapse free survival probabilities of GM-CSF^{High} TNF^{High} IFN-γ^{High} (n=22) and GM-CSF^{Low} TNF^{Low} IFN-γ^{Low} (n=19) COG P9906 B-ALL patients (GSE11877). (FIG. 7E) Comparison of overall survival probabilities of CyTOF B/T-ALL patients (n=20) divided into two groups based on the median frequencies of GM-CSF⁺, TNF⁺, and IFN-γ⁺ NK cells as ‘High GM-CSF⁺ TNF⁺ IFN-γ⁺ NK’ (n=7), and ‘Low GM-CSF⁺ TNF⁺ IFN-γ⁺ NK’ (n=8). (FIGS. 7F-7G) Stacked bar charts comparing the proportions of ‘High GM-CSF⁺ TNF⁺ IFN-γ⁺ NK’ (n=7) and ‘Low GM-CSF⁺ TNF⁺ IFN-γ⁺ NK’ (n=8) CyTOF B/T-ALL patients with WBC>100,000 (FIG. 7F), and those who survived (FIG. 7G). Survival curves using the Kaplan-Meier method are displayed. For survival curves, exact p-values were calculated using the log-rank test. For all other analyses, exact p-values were calculated using Mann-Whitney test, ns=not significant.

FIGS. 8A-8F show increased cell death and reduced proliferation are not responsible for reduced NK frequencies in the B/T-ALL microenvironment. (FIGS. 8A-8C) Proportion of dead cells in Total NK cells and NK subsets in BMMC (FIG. 8A), and PBMC (FIG. 8B), and representative dot plots (FIG. 8C), of healthy donors (n=11 BMMC, n=10 PBMC) and B/T-ALL patients (n=9 BMMC, n=8 PBMC) calculated by CyTOF. (FIGS. 8D-8F) Proportion of Ki-67⁺ proliferating total NK cells and NK subsets in BMMC (FIG. 8D), and PBMC (FIG. 8E), and representative dot plots (FIG. 8F), of healthy donors (n=5 BMMC, n=4 PBMC) and B/T-ALL patients (n=8 BMMC, n=9 PBMC) calculated by CyTOF. Graphs show median±interquartile range. Exact p-values have been calculated using the Mann-Whitney test, ns=non-significant.

FIG. 9A-9B show changes in frequencies of CD56^{bright} and CD56^{dim} NK cells in high-risk B/T-ALL. (FIG. 9A) Comparison of percentages of CD56^{bright} NK and CD56^{dim} NK cells in BMMC of healthy donors (n=12) and B/T-ALL patients (n=12) by CyTOF. (FIG. 9B) Comparison of percentages of CD56^{bright} NK, and CD56^{dim} NK cells in PBMC of healthy donors (n=10) and B/T-ALL patients (n=8). All NK subsets are shown as percentages of total NK cells. Graphs show median±interquartile range. Exact p-values were calculated using the Mann-Whitney test. ns=non-significant.

FIGS. 10A-10F show changes in NKp46, CD27 and CD57 expression in unstimulated NK cells between B/T-ALL patients and healthy donors. (FIGS. 10A-10B) CD27 vs NKp46 expression analysis by CyTOF in total NK cells of BMMC (FIG. 10A) and PBMC (FIG. 10B) of B/T-ALL patients (BMMC, n=12; PBMC, n=8) compared to healthy individuals (BMMC, n=12; PBMC, n=10). Graphs show mean±SEM for each subset. (FIGS. 10C-10D) Representative dot plots of the expression of CD27 vs NKp46 on total NK cells in BMMC (FIG. 10C) and PBMC (FIG. 10D) of healthy control and B/T-ALL patients. (FIGS. 10E-10F) CD57 expression on subsets defined by CD27 (FIG. 10E) and NKp46 (FIG. 10F) expression as described in (FIGS. 10A-10B). Graphs show median±interquartile range. Exact p-values were calculated using the Mann-Whitney test, ns=not significant.

FIG. 11 shows the gating strategy for flow cytometry analysis of PBMC. From the lymphocyte cluster; singlets were gated followed by selection of live (Ghost-UV450⁻) and CD45⁺ populations. Monocytes were then gated out (CD14− gate), followed by the non-B and non-T cell gate (CD19⁻CD3⁻). From non-B non-T and non-monocyte populations, CD56⁺ NK cells were selected for downstream gating of surface markers.

FIGS. 12A-12F show cytokine production in stimulated BMMC and PBMC NK cells of B/T-ALL patients. Frequencies of PMA/Ionomycin-stimulated cells expressing MIP-1β (FIG. 12A) and IL-2 (FIG. 12B) in total NK and NK subsets in BMMC, and expressing MIP-1β (FIG. 12C) and IL-2 (FIG. 12D) in total NK and NK subsets in and PBMC (FIGS. 12C-12D) of B/T-ALL patients (BMMC, n=12; PBMC, n=8) and healthy donors (BMMC, n=12; PBMC, n=10). (FIGS. 12E-12F) Representative dot plots for stimulated total NK cells expressing MIP-1β and IL-2 in BMMC (FIG. 12E) and PBMC (FIG. 12F) of B/T-ALL patients. Graphs show median±interquartile range. Exact p-values were calculated using Mann-Whitney test, ns=not significant.

FIGS. 13A-13J show comparison of cytotoxic granules and degranulation of NK cells between B/T-ALL patients and healthy donors. (FIGS. 13A-13B) Frequencies of cells expressing perforin (PRF, (FIG. 13A)) and granzyme B (GZMB, (FIG. 13B)) in unstimulated total NK cells and NK subsets in BMMC of B/T-ALL patients (n=12) and heathy donors (n=12). (FIG. 13C) Representative density plots showing PRF and GZMB vs CD56 expression in unstimulated total NK cells in BMMC of B/T-ALL patients and heathy donors. (FIGS. 13D-13E) Frequencies of cells expressing PRF (FIG. 13D) and GZMB (FIG. 13E) in unstimulated total NK cells and NK subsets in PBMC of B/T-ALL patients (n=8) and heathy donors (n=10). (FIG. 13F) Representative density plots showing PRF and GZMB vs CD56 expression in unstimulated total NK cells in PBMC of B/T-ALL patients and heathy donors. (FIGS. 13G-13J) Comparison of frequencies (FIG. 13G) and representative density plots (FIG. 13H) of PMA/Ionomycin-stimulated NK cell subsets expressing CD107a in BMMC, and frequencies (FIG. 13I) and representative density plots (FIG. 13J) of PMA/Ionomycin-stimulated NK cell subsets expressing CD107a in PBMC (FIGS. 131-13J) of healthy donors (BMMC: n=12, PBMC: n=10) and B/T-ALL patients (BMMC: n=12, PBMC: n=8) by CyTOF. Graphs show median±interquartile range. Exact p-values were calculated using Mann-Whitney test, ns=not significant.

FIGS. 14A-14K show comparison of expression of immune checkpoints in BMMC of ALL patients and healthy donors. (FIGS. 14A-14K) Comparison of the proportions of total NK cells or CD56^{bright}, CD56^{dim} NK subsets expressing CTLA-4 (FIG. 14A), PD-1 (FIG. 14B), ICOS (FIG. 14C), PD-L1 (FIG. 14D), PD-L2 (FIG. 14E), ILT2 (FIG. 14F), LAG-3 (FIG. 14G), KLRG1 (FIG. 14H), TIM-3 (FIG. 14I), TIGIT (FIG. 14J), and Siglec-7 (FIG. 14K) in healthy donors (n=5) and B/T-ALL patients (n=8) by CyTOF. Graphs show median±interquartile range. Exact p-values were calculated using Mann-Whitney test, ns=not significant.

FIGS. 15A-15G show comparison of expression of immune checkpoints in PBMC of ALL patients and healthy donors. Comparison of the proportions of Total NK cells or CD56^{bright}, CD56^{dim} NK subsets expressing CTLA-4 (FIG. 15A), PD-1 (FIG. 15B), ICOS (FIG. 15C), PD-L1 (FIG. 15D), TIGIT (FIG. 15E), ILT2 (FIG. 15F), and TIM-3 (FIG. 15G) in healthy donors (n=4) and B/T-ALL patients (n=9) by CyTOF. Graphs show median±interquartile range. Exact p-values were calculated using Mann-Whitney test, ns=not significant.

FIG. 16 shows gene expression signature of resting and activated NK subsets in LM22 CIBERSORT reference matrix. List of differentially regulated genes (≥2-fold) between resting and activated NK subsets in LM22 reference matrix used for CIBERSORT to deconvolute COG B-ALL data in FIGS. 6A-6G.

FIGS. 17A-17C show poorly prognostic ALL patients with only activated NK cells exhibit chronic NK activation. Comparison of transcript levels of CD56 (FIG. 17A) and CD69 NK activation marker (FIG. 17B) between COG P9906 B-ALL patients with only activated NK (n=85) or only resting NK (n=73) cells. Exact p-values were calculated using Mann-Whitney test. (FIG. 17C) Gene set enrichment analysis (GSEA) showing differential regulation of the calcium (Ca²⁺) signaling pathway between COG P9906 BALL patients with 100% activated NK (n=85) or 100% resting NK (n=73) cells. Exact p-values were calculated in GSEA.

FIGS. 18A-18H show NK cell phenotype and functions are similarly altered in pediatric and adult ALL. Comparison by CyTOF analysis of the frequencies of unstimulated NK cell subsets in BMMC from healthy controls (n=12), pediatric B/T-ALL (n=9) and adult B/T-ALL (n=3). Total NK cells as non-monocytes, non-T, non-B, CD56⁺ cells (FIG. 18A); CD56^{bright} (FIG. 18B) and CD56^{dim} NK (FIG. FIG. 18C) subsets; CD56^{bright}CD27⁺, CD56^{bright}CD27 CD56^{dim}CD27−, CD56^{dim}CD27⁺NK fractions (FIG. 18D); and CD27⁺NKp46⁺, CD27⁻NKp46, CD27⁻NKp46, CD27-NKp46⁺NK fractions (FIG. 18E). (FIGS. 18F-18G) Comparison by CyTOF analysis of the frequencies of (FIG. 18F) GM-CSF⁺, (FIG. 18G) TNF-α⁺, and (FIG. 18H) IFN-γ⁺ NK cell subsets in BMMC from healthy controls (n=12), pediatric B/T-ALL (n=9) and adult B/T-ALL (n=3) after stimulation by PMA/Ionomycin for 4 hours. Graphs show median±interquartile range. Exact p-values were calculated using Mann-Whitney test, ns=not significant.

FIGS. 19A-19B illustrate CXCR4 expression on NK cells. Live PBMCs and bone marrow cells were gated followed by selection of NK cells in non-CD3 and non-CD14 cell compartments. Flow cytometric analysis of PBMCs (n=10) (FIG. 19A) and bone marrow (n=3) (FIG. 19B) of HD and B-ALL patients showing the frequencies of CXCR4 positive NK cells. Exact P values were calculated using the Mann-Whitney U test.

FIGS. 20A-20B. show MHC-I expression on B and T cells in PBMCs of HD and B-ALL patients. (FIG. 20A) Flow cytometric analysis showing frequencies of HLA-ABC positive CD19⁺in PBMCS of HD and B-ALL patients, n=10. (FIG. 20B) Dot plot showing the median fluorescence intensity of HLA-ABC on CD3⁺ cells in the PBMCs of HD and B-ALL patients, n=10. Exact P values were calculated using the Mann-Whitney U test.

FIGS. 21A-21B. illustrate specific cytotoxicity analysis of CRISPRa IL-15 and IL-15+type I interferons (IFN-Is) secreting NK cells against healthy PBMCs. (FIG. 21A) Flow cytometric analysis showing specific cytotoxicity of IL-15 NK cells against three healthy donors PBMCs. (FIG. 21B) Flow cytometric analysis showing specific cytotoxicity of IL-15+IFN-Is NK cells against three healthy donors PBMCs. Error bar represents mean±SEM of experimental triplicates values.

FIGS. 22A-22B. Specific cytotoxicity analysis of CRISPRa IL-15 and IL-15+type I interferons (IFN-Is) secreting NK cells against ALL cell lines. (FIG. 22A) Flow cytometric analysis showing specific cytotoxicity of IL-15 NK cells against three different ALL cell lines namely K562 (chronic myelogenous leukemia), SEM (high risk B-ALL) and KOPN8 (high risk B-ALL). (FIG. 22B) Flow cytometric analysis showing specific cytotoxicity of IL-15+IFN-Is NK cells against three different ALL cell lines namely K562 (chronic myelogenous leukemia), SEM (high risk B-ALL) and KOPN8 (high risk B-ALL). Error bar represents mean±SEM of experimental triplicates values. The exact P value was calculated using the unpaired Student t test.

### DETAILED DESCRIPTION

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc. References identified herein are expressly incorporated herein by reference in their entireties unless otherwise indicated.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non-limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. For example, the nucleic acid provided herein may be part of a vector. For example, the nucleic acid provided herein may be part of an adenoviral vector, which may be transduced into a cell. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the TUPAC-TUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins (1984)).

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In embodiments, the amino acid side chain may be a non-natural amino acid side chain. In embodiments, the amino acid side chain is H,

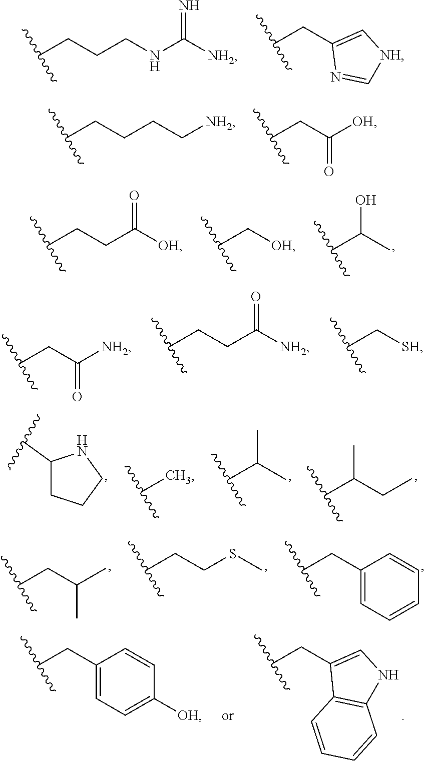

An amino acid residue in a protein “corresponds” to a given residue when it occupies the same essential structural position within the protein as the given residue.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

For specific proteins described herein, the named protein includes any of the protein's naturally occurring forms, variants or homologs that maintain the protein activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The term “CD56 protein” or “CD56” as used herein includes any of the recombinant or naturally-occurring forms of CD56 protein, also known as Neural cell adhesion molecule 1, N-CAM-1, or variants or homologs thereof that maintain CD56 activity (e.g. within at least 50%, 80%, 90%, 95%, 96%, 97%, 98%, 99% or 100% activity compared to CD56). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD56 protein. In embodiments, the CD56 protein is substantially identical to the protein identified by the UniProt reference number P13591 or a variant or homolog having substantial identity thereto.

The term “CD94 protein” or “CD94” as used herein includes any of the recombinant or naturally-occurring forms of CD94 protein, also known as Natural killer cells antigen CD94, or variants or homologs thereof that maintain CD94 activity (e.g. within at least 50%, 80%, 90%, 95%, 96%, 97%, 98%, 99% or 100% activity compared to CD94). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD94 protein. In embodiments, the CD94 protein is substantially identical to the protein identified by the UniProt reference number Q13241 or a variant or homolog having substantial identity thereto.

The term “CD11b protein” or “CD11b” as used herein includes any of the recombinant or naturally-occurring forms of CD11b protein, also known as CD11 antigen-like family member 13, CR-3 alpha chain, Cell surface glycoprotein MAC-1 subunit alpha, Leukocyte adhesion receptor MO1, or variants or homologs thereof that maintain CD11b activity (e.g. within at least 50%, 80%, 90%, 95%, 96%, 97%, 98%, 99% or 100% activity compared to CD11b). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD11b protein. In embodiments, the CD11b protein is substantially identical to the protein identified by the UniProt reference number P11215 or a variant or homolog having substantial identity thereto.

The term “CD27 protein” or “CD27” as used herein includes any of the recombinant or naturally-occurring forms of CD27 protein, also known as CD27L receptor, T-cell activation antigen CD27, Tumor necrosis factor receptor superfamily member 7 or variants or homologs thereof that maintain CD27 activity (e.g. within at least 50%, 80%, 90%, 95%, 96%, 97%, 98%, 99% or 100% activity compared to CD27). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD27 protein. In embodiments, the CD27 protein is substantially identical to the protein identified by the UniProt reference number P26842 or a variant or homolog having substantial identity thereto.

The term “CD69 protein” or “CD69” as used herein includes any of the recombinant or naturally-occurring forms of CD69 protein, also known as Early activation antigen CD69, EA1 or variants or homologs thereof that maintain CD69 activity (e.g. within at least 50%, 80%, 90%, 95%, 96%, 97%, 98%, 99% or 100% activity compared to CD69). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD69 protein. In embodiments, the CD69 protein is substantially identical to the protein identified by the UniProt reference number Q07108 or a variant or homolog having substantial identity thereto.

The term “Interferon gamma protein” or “Interferon gamma” as used herein includes any of the recombinant or naturally-occurring forms of Interferon gamma protein (IFN-γ), also known as Immune interferon or variants or homologs thereof that maintain IFN-γ activity (e.g. within at least 50%, 80%, 90%, 95%, 96%, 97%, 98%, 99% or 100% activity compared to IFN-γ). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IFN-γ protein. In embodiments, the IFN-γ protein is substantially identical to the protein identified by the UniProt reference number P01579 or a variant or homolog having substantial identity thereto.

The term “Tumor necrosis factor protein” or “Tumor necrosis factor” as used herein includes any of the recombinant or naturally-occurring forms of Tumor necrosis factor protein (TNF), also known as Tumor necrosis factor ligand superfamily member 2 or variants or homologs thereof that maintain TNF activity (e.g. within at least 50%, 80%, 90%, 95%, 96%, 97%, 98%, 99% or 100% activity compared to TNF). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TNF protein. In embodiments, the TNF protein is substantially identical to the protein identified by the UniProt reference number P01375 or a variant or homolog having substantial identity thereto.

The term “Granulocyte-macrophage colony-stimulating factor protein” or “Granulocyte-macrophage colony-stimulating factor” as used herein includes any of the recombinant or naturally-occurring forms of Granulocyte-macrophage colony-stimulating factor protein (GM-CSF), also known as Cachectin or variants or homologs thereof that maintain GM-CSF activity (e.g. within at least 50%, 80%, 90%, 95%, 96%, 97%, 98%, 99% or 100% activity compared to GM-CSF). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring GM-CSF protein. In embodiments, the GM-CSF protein is substantially identical to the protein identified by the UniProt reference number P04141 or a variant or homolog having substantial identity thereto.

The term “Interleukin-2 protein” or “Interleukin-2” as used herein includes any of the recombinant or naturally-occurring forms of Interleukin-2 protein (IL-2), also known as Cachectin or variants or homologs thereof that maintain IL-2 activity (e.g. within at least 50%, 80%, 90%, 95%, 96%, 97%, 98%, 99% or 100% activity compared to IL-2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-2 protein. In embodiments, the IL-2 protein is substantially identical to the protein identified by the UniProt reference number P60568 or a variant or homolog having substantial identity thereto.

The term “DNAX Accessory Molecule-1 protein” or “DNAX Accessory Molecule-1” as used herein includes any of the recombinant or naturally-occurring forms of DNAX Accessory Molecule-1 protein (DNAM 1), also known as CD226 antigen, or variants or homologs thereof that maintain DNAM 1 activity (e.g. within at least 50%, 80%, 90%, 95%, 96%, 97%, 98%, 99% or 100% activity compared to DNAM 1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring DNAM 1 protein. In embodiments, the DNAM 1 protein is substantially identical to the protein identified by the UniProt reference number Q15762 or a variant or homolog having substantial identity thereto.

The term “Killer cell immunoglobulin-like receptor 2DL1 protein” or “Killer cell immunoglobulin-like receptor 2DL1” as used herein includes any of the recombinant or naturally-occurring forms of Killer cell immunoglobulin-like receptor 2DL1 protein (KIR2DL1), also known as CD158 antigen-like family member A, Natural killer-associated transcript 1 or variants or homologs thereof that maintain KIR2DL1 activity (e.g. within at least 50%, 80%, 90%, 95%, 96%, 97%, 98%, 99% or 100% activity compared to KIR2DL1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring KIR2DL1 protein. In embodiments, the KIR2DL1 protein is substantially identical to the protein identified by the UniProt reference number P43626 or a variant or homolog having substantial identity thereto.

The term “Siglec-7 protein” or “Siglec-7” as used herein includes any of the recombinant or naturally-occurring forms of Siglec-7, also known as Sialic acid-binding Ig-like lectin 7, Adhesion inhibitory receptor molecule 1 or variants or homologs thereof that maintain Siglec-7 activity (e.g. within at least 50%, 80%, 90%, 95%, 96%, 97%, 98%, 99% or 100% activity compared to Siglec-7). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Siglec-7 protein. In embodiments, the Siglec-7 protein is substantially identical to the protein identified by the UniProt reference number Q9Y286 or a variant or homolog having substantial identity thereto.

The term “Perforin protein” or “Perforin” as used herein includes any of the recombinant or naturally-occurring forms of perforin (PRY), also known as P1, Cytolysin or variants or homologs thereof that maintain perforin activity (e.g. within at least 50%, 80%, 90%, 95%, 96%, 97%, 98%, 99% or 100% activity compared to perforin). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring perforin protein. In embodiments, the perforin protein is substantially identical to the protein identified by the UniProt reference number P14222 or a variant or homolog having substantial identity thereto.

The term “granzyme protein” or “granzyme” as used herein includes any of the recombinant or naturally-occurring forms of granzyme (GZMB), also known as P1, Cytolysin or variants or homologs thereof that maintain granzyme activity (e.g. within at least 50%, 80%, 90%, 95%, 96%, 97%, 98%, 99% or 100% activity compared to granzyme). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring granzyme protein. In embodiments, the perforin protein is substantially identical to the protein identified by the UniProt reference number P10144 or a variant or homolog having substantial identity thereto.

The term “CD107a protein” or “CD107a” as used herein includes any of the recombinant or naturally-occurring forms of CD107a, also known as Lysosome-associated membrane glycoprotein 1 or variants or homologs thereof that maintain CD107a activity (e.g. within at least 50%, 80%, 90%, 95%, 96%, 97%, 98%, 99% or 100% activity compared to CD107a). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD107a protein. In embodiments, the perforin protein is substantially identical to the protein identified by the UniProt reference number P11279 or a variant or homolog having substantial identity thereto.

The term “Lymphocyte-activation gene 3 protein” or “Lymphocyte-activation gene 3” as used herein includes any of the recombinant or naturally-occurring forms of Lymphocyte-activation gene 3 (LAG-3), also known as CD223 or variants or homologs thereof that maintain LAG-3 activity (e.g. within at least 50%, 80%, 90%, 95%, 96%, 97%, 98%, 99% or 100% activity compared to LAG-3). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring LAG-3 protein. In embodiments, the perforin protein is substantially identical to the protein identified by the UniProt reference number P18627 or a variant or homolog having substantial identity thereto.

The term “Killer cell lectin-like receptor subfamily G member 1 protein” or “Killer cell lectin-like receptor subfamily G member 1” as used herein includes any of the recombinant or naturally-occurring forms of Killer cell lectin-like receptor subfamily G member 1 (KLRG1), also known as C-type lectin domain family 15 member A, MAFA-like receptor, or variants or homologs thereof that maintain KLRG1 activity (e.g. within at least 50%, 80%, 90%, 95%, 96%, 97%, 98%, 99% or 100% activity compared to KLRG1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring KLRG1 protein. In embodiments, the perforin protein is substantially identical to the protein identified by the UniProt reference number Q96E93 or a variant or homolog having substantial identity thereto.

The term “Programmed cell death 1 ligand 2 protein” or “Programmed cell death 1 ligand 2” as used herein includes any of the recombinant or naturally-occurring forms of Programmed cell death 1 ligand 2 (PD-L2), also known as PD-1 ligand 2, CD273, or variants or homologs thereof that maintain PD-L2 activity (e.g. within at least 50%, 80%, 90%, 95%, 96%, 97%, 98%, 99% or 100% activity compared to PD-L2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PD-L2 protein. In embodiments, the perforin protein is substantially identical to the protein identified by the UniProt reference number Q9BQ51 or a variant or homolog having substantial identity thereto.

The term “CXCR4 protein” or “CXCR4” as used herein includes any of the recombinant or naturally-occurring forms of CXCR4, also known as C-X-C chemokine receptor type 4 or variants or homologs thereof that maintain CXCR4 activity (e.g. within at least 50%, 80%, 90%, 95%, 96%, 97%, 98%, 99% or 100% activity compared to CXCR4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CXCR4 protein. In embodiments, the CXCR4 protein is substantially identical to the protein identified by the UniProt reference number P61073 or a variant or homolog having substantial identity thereto.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“B Cells” or “B lymphocytes” refer to their standard use in the art. B cells are lymphocytes, a type of white blood cell (leukocyte), that develops into a plasma cell (a “mature B cell”), which produces antibodies. An “immature B cell” is a cell that can develop into a mature B cell. Generally, pro-B cells undergo immunoglobulin heavy chain rearrangement to become pro B pre B cells, and further undergo immunoglobulin light chain rearrangement to become an immature B cells. Immature B cells include T1 and T2 B cells.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

A “memory T cell” is a T cell that has previously encountered and responded to its cognate antigen during prior infection, encounter with cancer or previous vaccination. At a second encounter with its cognate antigen memory T cells can reproduce (divide) to mount a faster and stronger immune response than the first time the immune system responded to the pathogen.

A “regulatory T cell” or “suppressor T cell” is a lymphocyte which modulates the immune system, maintains tolerance to self-antigens, and prevents autoimmune disease.

As used herein, the terms “natural killer cells” and “NK cells” are used in accordance with their plain ordinary meaning and refer to a type of cytotoxic lymphocyte involved in the innate immune system. The role NK cells play is typically analogous to that of cytotoxic T cells in the vertebrate adaptive immune response. NK cells may provide rapid responses to virus-infected cells, acting at around 3 days after infection, and respond to tumor formation. Typically, immune cells detect major histocompatibility complex (MHC) presented on infected cell surfaces, triggering cytokine release, causing lysis or apoptosis. NK cells typically have the ability to recognize stressed cells in the absence of antibodies and MHC, allowing for a much faster immune reaction. In embodiments, NK cells are identified by the presence of CD56 and the absence of CD3. NK cells may be capable of recognizing and killing stressed cells in the absence of antibodies and MHC.

NK cells typically undergo five developmental stages from hematopoetic stem cells. Within human bone marrow, NK cells typically originate from Lin-CD34+CD133+CD244+ multipotent hematopoietic stem cells (HSCs) that commit to the lymphoid lineage to become CD45RA+CD133+ lymphoid primed multipotent progenitors (LMPPs). 13 LMPPs differentiate into common lymphoid progenitors (CLP) marked by CD38+CD7+CD10+CD127+ that have the potential to become Pro-B, Pre-T, or ILCs, apart from CD7+, CD127+(IL-7Rα+), CD122+(IL-2Rβ+), CD117+(c-Kit+), and IL-1R1^{low} Stage 1 NK progenitors (NKPs). NKPs transition into Stage 2 Pre-NK cells marked by the expression of CD7+CD127+ and down-regulation of CD3. Stage 2 Pre-NK cells may be divided into Stage 2a and 2b substages, based on whether they lack or express IL-1R1, respectively. The acquisition of activating receptors such as NKG2D(CD314), NKp46(CD335), NKp30(CD337), and CD161 marks the transition from Stage 2b Pre-NK cells into Stage 3 immature NK cells (iNK) cells. iNK cells develop into Stage 4 CD56^{bright} NK cells which are divided into substages 4a and 4b, with the latter distinct from the former by the expression of NKp80. CD56^{bright} NK cells eventually develop into Stage 5 CD56^{dim} mature NK (mNK) cells by the gradual up-regulation of CD94/NKG2C and CD16(FcγRIII), and by the down-regulation of CD56, c-Kit(CD117), and CD94/NKG2A. Stage 4 cells (CD56^{bright}) develop to fully mature Stage 5 cells (CD56^{dim}) in a healthy individual and possess distinct markers that distinguish them.

A “regulatory T cell” or “suppressor T cell” is a lymphocyte which modulates the immune system, maintains tolerance to self-antigens, and prevents autoimmune disease.

“Allogeneic” is used in accordance with its plain and ordinary meaning and includes cells or tissues derived from different individuals of the same species. The term “allogeneic transplant” or “allogeneic transfusion” refers to the transfer of biological material to a recipient from a genetically non-identical donor of the same species. For example, an allogeneic transplant may include transfer of tissue, cells or an organ to a recipient that is genetically non-identical to the donor. In embodiments, the allogeneic cells are allogeneic NK cells.

“Autologous” is used in accordance with its plain and ordinary meaning and includes cells or tissues derived from the same individual. In embodiments, the autologous cells are autologous NK cells. An autolous NK cell may be taken from an individual and genetically modified (e.g. nucleic acid integrated into NK cell genome) before being put back into the same individual.

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

“Marker” or “biomarker” is used in accordance with its plain ordinary meaning and refers to a measurable substance or compound in an biological sample that is indicative of a process or of a condition or a disease. A marker may be indicative of how well a subject responds to a treatment for a disease In embodiments, the marker is CD56, CD94, CD69, a cytokine or calcium (Ca²⁺) mobilization. In embodiments, the marker is a CD11b⁻CD27− NK cell or a CD11b⁺CD27− NK cell.

As defined herein, the term “activation”, “activate”, “activating”, “activator” and the like in reference to a protein-inhibitor interaction means positively affecting (e.g., increasing) the activity or function of the protein relative to the activity or function of the protein in the absence of the activator. In embodiments activation means positively affecting (e.g., increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist. In embodiments, the agonist increases expression or activity of a protein relative to the expression or activity of the protein in the absence of the agonist.

As defined herein, the term “inhibition”, “inhibit”, “inhibiting” and the like in reference to a protein-inhibitor interaction means negatively affecting (e.g., decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor binds to the target protein). In embodiments, inhibition refers to a reduction of activity of a target protein from an indirect interaction (e.g., an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g., a protein associated disease, a cancer (e.g., leukemia)) means that the disease (e.g., cancer) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “signaling pathway” as used herein refers to a series of interactions between cellular and optionally extra-cellular components (e.g., proteins, nucleic acids, small molecules, ions, lipids) that conveys a change in one component to one or more other components, which in turn may convey a change to additional components, which is optionally propagated to other signaling pathway components.

The terms “disease” or “condition” refer to a state of being or health status of a patient or subject capable of being treated with the compounds or methods provided herein. The disease may be a cancer. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

“Acute lymphoblastic leukemia” or “ALL” refers to a cancer of the blood and bone marrow characterized by production of immature lymphocytes, a type of white blood cell, rather than mature lymphocytes. Lymphocytes include NK cells, T cells and B cells. The immature lymphocytes develop into pathogenic white blood cells referred to as “lymphoblasts”. The production of lymphoblasts crowd out healthy red blood cells, white blood cells, and platelets. Symptoms of ALL may include exhaustion, pale skin color, fever, easy bleeding or bruising, enlarged lymph nodes, or bone pain. ALL progresses rapidly and is typically fatal if left untreated. In embodiments, the ALL is B-cell ALL or T-cell ALL.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The term “healthy patient” or “healthy subject” as used herein refers to a subject that does not have cancer. Provided herein are methods of treating or preventing infectious disease in a cancer patient. In embodiments, the cancer is leukemia. As used herein, the healthy subject does not have leukemia. In embodiments, the healthy subject does not have ALL. In embodiments, the healthy subject does not have an infectious disease.

A “effective amount” or “therapeutically effective amount” are used interchangeably and refer to an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). For any composition (e.g. NK cell composition) described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” is used in accordance with its plain and ordinary meaning in the art and includes oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g., mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g., mice) and from humans.

### Methods of Identifying Dysfunctional NK Cells

The methods provided herein are useful for identifying dysfunctional NK cells in leukemia patients. As used herein, “dysfunctional NK cell” refers to an NK cell from a leukemia patient as identified by the current invention. For example, dysfunctional natural killer cells have (a) an elevated expression level of CD56 relative to a standard control; (b) an elevated expression level of CD94 relative to the standard control; (c) an elevated ratio of CD11b⁻CD27− NK cells to CD11b⁺CD27− NK cells relative to the standard control; (d) the presence of CD69; (e) the presence of a cytokine; and/or (f) calcium (Ca²⁺) mobilization. For example, dysfunctional natural killer cells have (a) an elevated expression level of CD56 relative to a standard control; (b) an elevated expression level of CD94 relative to the standard control; (c) an elevated ratio of CD11b⁻CD27− NK cells to CD11b⁺CD27− NK cells relative to the standard control; (d) the presence of CD69; (e) the presence of a cytokine; or (f) calcium (Ca²⁺) mobilization. For example, dysfunctional natural killer cells have (a) an elevated expression level of CD56 relative to a standard control; (b) an elevated expression level of CD94 relative to the standard control; (c) an elevated ratio of CD11b⁻CD27− NK cells to CD11b⁺CD27− NK cells relative to the standard control; (d) the presence of CD69; (e) the presence of a cytokine; and (f) calcium (Ca²⁺) mobilization. The elevated expression level of CD56 or CD94, elevated ratio of CD11b⁻CD27− NK cells to CD11b⁺CD27− NK cells, presence of CD69 or cytokine, and calcium mobilization are detected as described herein. In embodiments, a dysfunctional NK cell has decreased cell-killing capability relative to a stand and control.

For the methods provided herein, in embodiments, (a) elevated expression level of CD56; (b) elevated expression level of CD94; (c) elevated ratio of CD11b⁻CD27− NK cells to CD11b⁺CD27− NK cells; (d) presence of CD69; (e) presence of a cytokine; and (f) calcium (Ca²⁺) mobilization are detected in NK cells. Thus, in embodiments, the standard control is an NK cell obtained from a subject who does not have leukemia. In embodiments, the standard control is a population of NK cells obtained from a subject who does not have leukemia. In embodiments, the standard control is a population of NK cells are obtained from the bone marrow of a subject who does not have leukemia. In embodiments, the standard control is a population of NK cells are obtained from PBMC of a subject who does not have leukemia. In embodiments, the standard control is a stage 5 NK cell. In embodiments, the standard control is a population of stage 5 NK cells. In embodiments, the stage 5 NK cell or population of stage 5 NK cells are from a standard NK cell line (e.g. NK-92 or NK-101). In embodiments, the stage 5 NK cell or population of stage 5 NK cells are obtained from a subject who does not have leukemia.

In embodiments, the NK cells obtained from the subject who does not have leukemia are tissue-matched to the subject. The term “tissue matched” is used in accordance to its ordinary meaning in the art and refers to matching HLA type between a donor and a recipient of a transplant (e.g. a cell (e.g. NK cell), tissue or organ). In embodiments, the population NK cells obtained from the subject who does not have leukemia are from the same tissue type as the population of NK cells obtained from the subject. In embodiments, NK cells obtained from the subject who does not have leukemia and NK cells obtained from the subject are from PBMC. In embodiments, NK cells obtained from the subject who does not have leukemia and NK cells obtained from the subject are from bone marrow (BMMC).

In an aspect is provided a method of identifying dysfunctional natural killer (NK) cells in a population of NK cells obtained from a subject having leukemia, wherein the method includes detecting in the population of NK cells: (a) an elevated expression level of CD56 relative to a standard control; (b) an elevated expression level of CD94 relative to the standard control; (c) an elevated ratio of CD11b⁻CD27− NK cells to CD11b⁺CD27− NK cells relative to the standard control; (d) the presence of CD69; (e) the presence of a cytokine; or (f) calcium (Ca²⁺) mobilization; thereby identifying the dysfunctional NK cells. In embodiments, the method includes detecting in the population of NK cells a combination of: (a) an elevated expression level of CD56 relative to a standard control; (b) an elevated expression level of CD94 relative to the standard control; (c) an elevated ratio of CD11b-CD27− NK cells to CD11b⁺CD27− NK cells relative to the standard control; (d) the presence of CD69; (e) the presence of a cytokine; or (f) calcium (Ca²⁺) mobilization. In embodiments, the method includes detecting in the population of NK cells a combination of: (a) an elevated expression level of CD56 relative to a standard control; (b) an elevated expression level of CD94 relative to the standard control; (c) an elevated ratio of CD11b⁻CD27− NK cells to CD11b⁺CD27− NK cells relative to the standard control; (d) the presence of CD69; (e) the presence of a cytokine; and (f) calcium (Ca²⁺) mobilization. In embodiments, the method includes detecting in the population of NK cells an elevated expression level of CD56 relative to a standard control. In embodiments, the method includes detecting in the population of NK cells an elevated expression level of CD94 relative to the standard control. In embodiments, the method includes detecting in the population of NK cells an elevated an elevated ratio of CD11b⁻CD27− NK cells to CD11b⁺CD27− NK cells relative to the standard control. In embodiments, the method includes detecting in the population of NK cells the presence of CD69. In embodiments, the method includes detecting in the population of NK cells the presence of a cytokine. In embodiments, the method includes detecting in the population of NK cells calcium (Ca²⁺) mobilization.

In embodiments, the method includes detecting in the population of NK cells (a) an elevated expression level of CD56 relative to a standard control and (a) an elevated expression level of CD94 relative to the standard control; (b) an elevated level of CD11b⁻ CD27− NK cells to CD11b⁺CD27− NK cells relative to the standard control; (c) the presence of CD69; (e) the presence of a cytokine; or (d) calcium (Ca²⁺) mobilization; thereby identifying the dysfunctional NK cells. In embodiments, the method includes detecting in the population of NK cells an elevated expression level of CD56 relative to a standard control and an elevated expression level of CD94 relative to the standard control. In embodiments, the method includes detecting in the population of NK cells an elevated expression level of CD56 relative to a standard control and an elevated ratio of CD11b⁻CD27− NK cells to CD11b⁺CD27− NK cells relative to the standard control. In embodiments, the method includes detecting in the population of NK cells an elevated expression level of CD56 relative to a standard control and the presence of CD69. In embodiments, the method includes detecting in the population of NK cells an elevated expression level of CD56 relative to a standard control and the presence of a cytokine. In embodiments, the method includes detecting in the population of NK cells an elevated expression level of CD56 relative to a standard control and calcium (Ca²⁺) mobilization.

In embodiments, an elevated expression level of CD56 refers to a CD56 expression level that is higher than the CD56 expression level of a NK cell from a subject who does not have leukemia (e.g. standard control). In embodiments, an elevated expression level of CD56 refers to a CD56 expression level that is higher than the expression level of CD56 in a population of NK cells from a subject who does not have leukemia (e.g. standard control). In embodiments, an elevated expression level of CD56 refers to a CD56 expression level that is higher than the mean CD56 expression level in a population of NK cells obtained from a subject who does not have leukemia (e.g. standard control). NK cells having elevated CD56 expression may be referred to as “CD56^{bright}” NK cells. NK cells that do not have elevated CD56 expression may be referred to as “CD56^{dim}”, NK cells. In embodiments, the mean expression level of CD56 in a population of cells correlates to the mean fluorescent intensity (MFI) of the population of cells (e.g. a population of cells obtained a subject who does not have leukemia) when detected by cytometric methods. For example, a higher MFI correlates to a higher expression level of CD56. In embodiments, the mean expression level of CD56 in a population of cells is the mean level of CD56 mRNA transcript in a population of cells (e.g. a population of cells obtained a subject who does not have leukemia) when detected by sequencing methods (e.g. single cell sequencing).

In embodiments, an elevated expression level of CD56 refers to a CD56 expression level that is higher than the CD56 expression level of a stage 5 NK cell (e.g. standard control). In embodiments, an elevated expression level of CD56 refers to a CD56 expression level that is higher than the expression level of CD56 in a population of stage 5 NK cells (e.g. standard control). In embodiments, an elevated expression level of CD56 refers to a CD56 expression level that is higher than the mean CD56 expression level in a population of stage 5 NK cells (e.g. standard control).

In embodiments, an elevated expression level of CD56 is an increased frequency of cells (e.g. proportion of cells) having high CD56 expression (e.g. CD56^{bright} cells) relative to a standard control. In embodiments, the standard control is the frequency of cells having high CD56 expression in a population of cells obtained from a subject who does not have leukemia. High CD56 expression may be a level of expression greater than the mean expression level of CD56 in the population of NK cells from the subject who does not have leukemia. In embodiments, the expression level of CD56 (e.g. frequency of CD56^{bright} cells) in the population of NK cells is elevated by at least 5% relative to the standard control (e.g. frequency of CD56^{bright} cells in a population of NK cells from a subject without leukemia). In embodiments, the expression level of CD56 (e.g. frequency of CD56^{bright} cells) in the population of NK cells is elevated by at least 10% relative to the standard control. In embodiments, the expression level of CD56 (e.g. frequency of CD56^{bright} cells) in the population of NK cells is elevated by at least 15% relative to the standard control. In embodiments, the expression level of CD56 (e.g. frequency of CD56^{bright} cells) in the population of NK cells is elevated by at least 20% relative to the standard control. In embodiments, the expression level of CD56 (e.g. frequency of CD56^{bright} cells) in the population of NK cells is elevated by at least 25% relative to the standard control. In embodiments, the expression level of CD56 (e.g. frequency of CD56^{bright} cells) in the population of NK cells is elevated by at least 30% relative to the standard control. In embodiments, the expression level of CD56 (e.g. frequency of CD56^{bright} cells) in the population of NK cells is elevated by at least 35% relative to the standard control. In embodiments, the expression level of CD56 (e.g. frequency of CD56^{bright} cells) in the population of NK cells is elevated by at least 40% relative to the standard control. In embodiments, the expression level of CD56 (e.g. frequency of CD56^{bright} cells) in the population of NK cells is elevated by at least 45% relative to the standard control. In embodiments, the expression level of CD56 (e.g. frequency of CD56^{bright} cells) in the population of NK cells is elevated by at least 50% relative to the standard control. In embodiments, the expression level of CD56 (e.g. frequency of CD56^{bright} cells) in the population of NK cells is elevated by at least 55% relative to the standard control. In embodiments, the expression level of CD56 (e.g. frequency of CD56^{bright} cells) in the population of NK cells is elevated by at least 60% relative to the standard control. In embodiments, the expression level of CD56 (e.g. frequency of CD56^{bright} cells) in the population of NK cells is elevated by at least 65% relative to the standard control. In embodiments, the expression level of CD56 (e.g. frequency of CD56^{bright} cells) in the population of NK cells is elevated by at least 70% relative to the standard control. In embodiments, the expression level of CD56 (e.g. frequency of CD56^{bright} cells) in the population of NK cells is elevated by at least 75% relative to the standard control. In embodiments, the expression level of CD56 (e.g. frequency of CD56^{bright} cells) in the population of NK cells is elevated by at least 80% relative to the standard control. In embodiments, the expression level of CD56 (e.g. frequency of CD56^{bright} cells) in the population of NK cells is elevated by at least 85% relative to the standard control. In embodiments, the expression level of CD56 (e.g. frequency of CD56^{bright} cells) in the population of NK cells is elevated by at least 90% relative to the standard control. In embodiments, the expression level of CD56 (e.g. frequency of CD56^{bright} cells) in the population of NK cells is elevated by at least 95% relative to the standard control. In embodiments, the expression level of CD56 (e.g. frequency of CD56^{bright} cells) in the population of NK cells is elevated by at least 100% relative to the standard control.

In embodiments, the expression level of CD56 (e.g. frequency of CD56^{bright} cells) in the population of NK cells is elevated by at least 0.1× relative to the standard control (e.g. frequency of CD56^{bright} cells in a population of NK cells from a subject without leukemia). In embodiments, the expression level of CD56 (e.g. frequency of CD56^{bright} cells) in the population of NK cells is elevated by at least 0.5× relative to the standard control. In embodiments, the expression level of CD56 (e.g. frequency of CD56^{bright} cells) in the population of NK cells is elevated by at least 1× relative to the standard control. In embodiments, the expression level of CD56 (e.g. frequency of CD56^{bright} cells) in the population of NK cells is elevated by at least 2× relative to the standard control. In embodiments, the expression level of CD56 (e.g. frequency of CD56^{bright} cells) in the population of NK cells is elevated by at least 3× relative to the standard control. In embodiments, the expression level of CD56 (e.g. frequency of CD56^{bright} cells) in the population of NK cells is elevated by at least 4× relative to the standard control. In embodiments, the expression level of CD56 (e.g. frequency of CD56^{bright} cells) in the population of NK cells is elevated by at least 5× relative to the standard control. In embodiments, the expression level of CD56 (e.g. frequency of CD56^{bright} cells) in the population of NK cells is elevated by at least 6× relative to the standard control. In embodiments, the expression level of CD56 (e.g. frequency of CD56^{bright} cells) in the population of NK cells is elevated by at least 7× relative to the standard control. In embodiments, the expression level of CD56 (e.g. frequency of CD56^{bright} cells) in the population of NK cells is elevated by at least 8× relative to the standard control. In embodiments, the expression level of CD56 (e.g. frequency of CD56^{bright} cells) in the population of NK cells is elevated by at least 9× relative to the standard control. In embodiments, the expression level of CD56 (e.g. frequency of CD56^{bright} cells) in the population of NK cells is elevated by at least 10× relative to the standard control. In embodiments, the expression level of CD56 (e.g. frequency of CD56^{bright} cells) in the population of NK cells is elevated by at least 20× relative to the standard control. In embodiments, the expression level of CD56 (e.g. frequency of CD56^{bright} cells) in the population of NK cells is elevated by at least 30× relative to the standard control. In embodiments, the expression level of CD56 (e.g. frequency of CD56^{bright} cells) in the population of NK cells is elevated by at least 40× relative to the standard control. In embodiments, the expression level of CD56 (e.g. frequency of CD56^{bright} cells) in the population of NK cells is elevated by at least 50× relative to the standard control. In embodiments, the expression level of CD56 (e.g. frequency of CD56^{bright} cells) in the population of NK cells is elevated by at least 60× relative to the standard control. In embodiments, the expression level of CD56 (e.g. frequency of CD56^{bright} cells) in the population of NK cells is elevated by at least 70× relative to the standard control. In embodiments, the expression level of CD56 (e.g. frequency of CD56^{bright} cells) in the population of NK cells is elevated by at least 80× relative to the standard control. In embodiments, the expression level of CD56 (e.g. frequency of CD56^{bright} cells) in the population of NK cells is elevated by at least 90× relative to the standard control. In embodiments, the expression level of CD56 (e.g. frequency of CD56^{bright} cells) in the population of NK cells is elevated by at least 100× relative to the standard control. In embodiments, the expression level of CD56 (e.g. frequency of CD56^{bright} cells) in the population of NK cells is elevated by at least 150× relative to the standard control. In embodiments, the expression level of CD56 (e.g. frequency of CD56^{bright} cells) in the population of NK cells is elevated by at least 200× relative to the standard control.

In embodiments, an elevated expression level of CD94 refers to a CD94 expression level that is higher than the CD94 expression level of an NK cell from a subject who does not have leukemia (e.g. standard control). In embodiments, an elevated expression level of CD94 refers to a CD94 expression level that is higher than expression level of a population of NK cells from a subject who does not have leukemia (e.g. standard control). In embodiments, an elevated expression level of CD94 refers to a CD94 expression level that is higher than the mean CD94 expression level of a population of NK cells obtained from a subject who does not have leukemia (e.g. standard control). In embodiments, the mean expression level of CD94 in a population of cells correlates to the mean fluorescent intensity (MFI) of a population of cells when detected by cytometric methods. In embodiments, the mean expression level of CD94 in a population of cells is the mean CD94 transcript mRNA level of a population of cells when detected by sequencing methods (e.g. single cell sequencing). NK cells having elevated CD94 expression may be referred to as CD94^{high} cells. NK cells that do not have elevated CD94 expression may be referred to as CD94^{low} cells.

In embodiments, an elevated expression level of CD94 refers to a CD94 expression level that is higher than the CD94 expression level of a stage 5 NK cell (e.g. standard control). In embodiments, an elevated expression level of CD94 refers to a CD94 expression level that is higher than the expression level of CD94 in a population of stage 5 NK cells (e.g. standard control). In embodiments, an elevated expression level of CD94 refers to a CD94 expression level that is higher than the mean CD94 expression level in a population of stage 5 NK cells (e.g. standard control).

In embodiments, an elevated expression level of CD94 is an increased frequency of NK cells (e.g. proportion of NK cells) having high CD94 expression (e.g. CD94^{high} cells) relative to a standard control. In embodiments, the standard control is the frequency of NK cells having high CD94 expression in a population of NK cells obtained from a subject who does not have leukemia. High CD94 expression may be an expression level higher than the mean CD94 expression level from the population of cells obtained from the subject who does not have leukemia. In embodiments, the expression level of CD94 (e.g. frequency of CD94^{high} cells) in the population of NK cells is elevated by at least 5% relative to the standard control (e.g. frequency of CD94 cells in a population of NK cells from a subject without leukemia). In embodiments, the expression level of CD94 (e.g. frequency of CD94 cells) in the population of NK cells is elevated by at least 10% relative to the standard control. In embodiments, the expression level of CD94 (e.g. frequency of CD94^{high} cells) in the population of NK cells is elevated by at least 15% relative to the standard control. In embodiments, the expression level of CD94 (e.g. frequency of CD94^{high} cells) in the population of NK cells is elevated by at least 20% relative to the standard control. In embodiments, the expression level of CD94 (e.g. frequency of CD94^{high} cells) in the population of NK cells is elevated by at least 25% relative to the standard control. In embodiments, the expression level of CD94 (e.g. frequency of CD94^{high} cells) in the population of NK cells is elevated by at least 30% relative to the standard control. In embodiments, the expression level of CD94 (e.g. frequency of CD94^{high} cells) in the population of NK cells is elevated by at least 35% relative to the standard control. In embodiments, the expression level of CD94 (e.g. frequency of CD94^{high} cells) in the population of NK cells is elevated by at least 40% relative to the standard control. In embodiments, the expression level of CD94 (e.g. frequency of CD94^{high} cells) in the population of NK cells is elevated by at least 45% relative to the standard control. In embodiments, the expression level of CD94 (e.g. frequency of CD94^{high} cells) in the population of NK cells is elevated by at least 50% relative to the standard control. In embodiments, the expression level of CD94 (e.g. frequency of CD94^{high} cells) in the population of NK cells is elevated by at least 55% relative to the standard control. In embodiments, the expression level of CD94 (e.g. frequency of CD94^{high} cells) in the population of NK cells is elevated by at least 60% relative to the standard control. In embodiments, the expression level of CD94 (e.g. frequency of CD94^{high} cells) in the population of NK cells is elevated by at least 65% relative to the standard control. In embodiments, the expression level of CD94 (e.g. frequency of CD94^{high} cells) in the population of NK cells is elevated by at least 70% relative to the standard control. In embodiments, the expression level of CD94 (e.g. frequency of CD94^{high} cells) in the population of NK cells is elevated by at least 75% relative to the standard control. In embodiments, the expression level of CD94 (e.g. frequency of CD94^{high} cells) in the population of NK cells is elevated by at least 80% relative to the standard control. In embodiments, the expression level of CD94 (e.g. frequency of CD94^{high} cells) in the population of NK cells is elevated by at least 85% relative to the standard control. In embodiments, the expression level of CD94 (e.g. frequency of CD94^{high} cells) in the population of NK cells is elevated by at least 90% relative to the standard control. In embodiments, the expression level of CD94 (e.g. frequency of CD94^{high} cells) in the population of NK cells is elevated by at least 95% relative to the standard control. In embodiments, the expression level of CD94 (e.g. frequency of CD94 cells) in the population of NK cells is elevated by at least 100% relative to the standard control.

In embodiments, the expression level of CD94 (e.g. frequency of CD94^{high} cells) in the population of NK cells is elevated by at least 0.1× relative to the standard control (e.g. frequency of CD94 cells in a population of NK cells from a subject without leukemia). In embodiments, the expression level of CD94 (e.g. frequency of CD94 cells) in the population of NK cells is elevated by at least 0.5× relative to the standard control. In embodiments, the expression level of CD94 (e.g. frequency of CD94^{high} cells) in the population of NK cells is elevated by at least 1× relative to the standard control. In embodiments, the expression level of CD94 (e.g. frequency of CD94^{high} cells) in the population of NK cells is elevated by at least 2× relative to the standard control. In embodiments, the expression level of CD94 (e.g. frequency of CD94^{high} cells) in the population of NK cells is elevated by at least 3× relative to the standard control. In embodiments, the expression level of CD94 (e.g. frequency of CD94^{high} cells) in the population of NK cells is elevated by at least 4× relative to the standard control. In embodiments, the expression level of CD94 (e.g. frequency of CD94^{high} cells) in the population of NK cells is elevated by at least 5× relative to the standard control. In embodiments, the expression level of CD94 (e.g. frequency of CD94^{high} cells) in the population of NK cells is elevated by at least 6× relative to the standard control. In embodiments, the expression level of CD94 (e.g. frequency of CD94^{high} cells) in the population of NK cells is elevated by at least 7× relative to the standard control. In embodiments, the expression level of CD94 (e.g. frequency of CD94^{high} cells) in the population of NK cells is elevated by at least 8× relative to the standard control. In embodiments, the expression level of CD94 (e.g. frequency of CD94^{high} cells) in the population of NK cells is elevated by at least 9× relative to the standard control. In embodiments, the expression level of CD94 (e.g. frequency of CD94^{high} cells) in the population of NK cells is elevated by at least 10× relative to the standard control. In embodiments, the expression level of CD94 (e.g. frequency of CD94^{high} cells) in the population of NK cells is elevated by at least 20× relative to the standard control. In embodiments, the expression level of CD94 (e.g. frequency of CD94^{high} cells) in the population of NK cells is elevated by at least 30× relative to the standard control. In embodiments, the expression level of CD94 (e.g. frequency of CD94^{high} cells) in the population of NK cells is elevated by at least 40× relative to the standard control. In embodiments, the expression level of CD94 (e.g. frequency of CD94^{high} cells) in the population of NK cells is elevated by at least 50× relative to the standard control. In embodiments, the expression level of CD94 (e.g. frequency of CD94^{high} cells) in the population of NK cells is elevated by at least 60× relative to the standard control. In embodiments, the expression level of CD94 (e.g. frequency of CD94^{high} cells) in the population of NK cells is elevated by at least 70× relative to the standard control. In embodiments, the expression level of CD94 (e.g. frequency of CD94^{high} cells) in the population of NK cells is elevated by at least 80× relative to the standard control. In embodiments, the expression level of CD94 (e.g. frequency of CD94^{high} cells) in the population of NK cells is elevated by at least 90× relative to the standard control. In embodiments, the expression level of CD94 (e.g. frequency of CD94^{high} cells) in the population of NK cells is elevated by at least 100× relative to the standard control. In embodiments, the expression level of CD94 (e.g. frequency of CD94^{high} cells) in the population of NK cells is elevated by at least 150× relative to the standard control. In embodiments, the expression level of CD94 (e.g. frequency of CD94^{high} cells) in the population of NK cells is elevated by at least 200× relative to the standard control.

In embodiments, an elevated ratio of CD11b⁻CD27− NK cells to CD11b⁺CD27− NK cells refers to a higher frequency of CD11b⁻CD27− NK cells to CD11b⁺CD27− NK cells relative to the frequency of CD11b⁻CD27− NK cells to CD11b⁺CD27− NK cells in a population of NK cells obtained from a subject who does not have leukemia (e.g. standard control). In embodiments, the frequency of CD11b⁻CD27− NK cells to CD11b⁺CD27− NK cells is at least about 2% higher than the frequency of CD11b⁻CD27− NK cells to CD11b⁺CD27− NK cells in cells obtained from a subject who does not have leukemia. In embodiments, the frequency of CD11b⁻CD27− NK cells to CD11b⁺CD27− NK cells is at least about 4% higher than the frequency of CD11b⁻CD27− NK cells to CD11b⁺CD27− NK cells in cells obtained from a subject who does not have leukemia. In embodiments, the frequency of CD11b⁻CD27− NK cells to CD11b⁺CD27− NK cells is at least about 5% higher than the frequency of CD11b⁻CD27− NK cells to CD11b⁺CD27− NK cells in cells obtained from a subject who does not have leukemia. In embodiments, the frequency of CD11b⁻CD27− NK cells to CD11b⁺CD27− NK cells is at least about 6% higher than the frequency of CD11b-CD27− NK cells to CD11b⁺CD27− NK cells in NK cells obtained from a subject who does not have leukemia. In embodiments, the frequency of CD11b⁻CD27− NK cells to CD11b⁺CD27− NK cells is at least about 8% higher than the frequency of CD11b⁻CD27− NK cells to CD11b⁺CD27− NK cells in NK cells obtained from a subject who does not have leukemia. In embodiments, the frequency of CD11b⁻CD27− NK cells to CD11b⁺CD27− NK cells is at least about 10% higher than the frequency of CD11b⁻CD27− NK cells to CD11b⁺CD27− NK cells in NK cells obtained from a subject who does not have leukemia. In embodiments, the frequency of CD11b⁻CD27− NK cells to CD11b⁺CD27− NK cells is at least about 15% higher than the frequency of CD11b⁻CD27− NK cells to CD11b⁺CD27− NK cells in NK cells obtained from a subject who does not have leukemia. In embodiments, the frequency of CD11b⁻CD27− NK cells to CD11b⁺CD27− NK cells is at least about 20% higher than the frequency of CD11b⁻CD27− NK cells to CD11b⁺CD27− NK cells in NK cells obtained from a subject who does not have leukemia. In embodiments, the frequency of CD11b⁻CD27− NK cells to CD11b⁺CD27− NK cells is at least about 25% higher than the frequency of CD11b⁻ CD27− NK cells to CD11b⁺CD27− NK cells in NK cells obtained from a subject who does not have leukemia. In embodiments, the frequency of CD11b⁻CD27− NK cells to CD11b⁺CD27− NK cells is at least about 30% higher than the frequency of CD11b⁻CD27− NK cells to CD11b⁺CD27− NK cells in NK cells obtained from a subject who does not have leukemia. In embodiments, the frequency of CD11b⁻CD27− NK cells to CD11b⁺CD27− NK cells is at least about 35% higher than the frequency of CD11b⁻CD27− NK cells to CD11b⁺CD27− NK cells in NK cells obtained from a subject who does not have leukemia. In embodiments, the frequency of CD11b⁻CD27− NK cells to CD11b⁺CD27− NK cells is at least about 40% higher than the frequency in NK cells obtained from a subject who does not have leukemia. In embodiments, the frequency of CD11b⁻CD27− NK cells to CD11b⁺CD27− NK cells is at least about 45% higher than the frequency in NK cells obtained from a subject who does not have leukemia. In embodiments, the frequency of CD11b⁻CD27− NK cells to CD11b⁺CD27− NK cells is at least about 50% higher than the frequency in NK cells obtained from a subject who does not have leukemia. In embodiments, the frequency of CD11b⁻CD27− NK cells to CD11b⁺CD27− NK cells is at least about 55% higher than the frequency in NK cells obtained from a subject who does not have leukemia. In embodiments, the frequency of CD11b⁻CD27− NK cells to CD11b⁺CD27− NK cells is at least about 60% higher than the frequency in NK cells obtained from a subject who does not have leukemia. In embodiments, the frequency of CD11b⁻CD27− NK cells to CD11b⁺CD27− NK cells is at least about 65% higher than the frequency in NK cells obtained from a subject who does not have leukemia. In embodiments, the frequency of CD11b⁻CD27− NK cells to CD11b⁺CD27− NK cells is at least about 70% higher than the frequency in NK cells obtained from a subject who does not have leukemia. In embodiments, the frequency of CD11b⁻CD27− NK cells to CD11b⁺CD27− NK cells is at least about 75% higher than the frequency in NK cells obtained from a subject who does not have leukemia. In embodiments, the frequency of CD11b⁻CD27− NK cells to CD11b⁺CD27− NK cells is at least about 80% higher than the frequency in NK cells obtained from a subject who does not have leukemia. In embodiments, the frequency of CD11b⁻CD27− NK cells to CD11b⁺CD27− NK cells is at least about 85% higher than the frequency in NK cells obtained from a subject who does not have leukemia. In embodiments, the frequency of CD11b⁻CD27− NK cells to CD11b⁺CD27− NK cells is at least about 90% higher than the frequency in NK cells obtained from a subject who does not have leukemia. In embodiments, the frequency of CD11b⁻CD27− NK cells to CD11b⁺CD27− NK cells is at least about 95% higher than the frequency in NK cells obtained from a subject who does not have leukemia. In embodiments, the frequency of CD11b⁻CD27− NK cells to CD11b⁺CD27− NK cells is at least about 100% higher than the frequency in NK cells obtained from a subject who does not have leukemia.

In embodiments, the ratio of CD11b⁻CD27− NK cells to CD11b^{m}CD27− NK cells is at least about 0.1× higher than the ratio of CD11b⁻CD27− NK cells to CD11b^{m}CD27− NK cells in cells obtained from a subject who does not have leukemia. In embodiments, the ratio of CD11b⁻CD27− NK cells to CD11b^{m}CD27− NK cells is at least about 0.5× higher than the ratio of CD11b⁻CD27− NK cells to CD11b^{m}CD27− NK cells in cells obtained from a subject who does not have leukemia. In embodiments, the ratio of CD11b⁻CD27− NK cells to CD11b^{m}CD27− NK cells is at least about 1× higher than the ratio of CD11b⁻CD27− NK cells to CD11b^{m}CD27− NK cells in NK cells obtained from a subject who does not have leukemia. In embodiments, the ratio of CD11b⁻CD27− NK cells to CD11b^{m}CD27− NK cells is at least about 2× higher than the ratio of CD11b⁻CD27− NK cells to CD11b^{m}CD27− NK cells in NK cells obtained from a subject who does not have leukemia. In embodiments, the ratio of CD11b-CD27− NK cells to CD11b^{m}CD27− NK cells is at least about 3× higher than the ratio of CD11b⁻CD27− NK cells to CD11b^{m}CD27− NK cells in NK cells obtained from a subject who does not have leukemia. In embodiments, the ratio of CD11b⁻CD27− NK cells to CD11b^{m}CD27− NK cells is at least about 4× higher than the ratio of CD11b⁻CD27− NK cells to CD11b^{m}CD27− NK cells in NK cells obtained from a subject who does not have leukemia. In embodiments, the ratio of CD11b⁻CD27− NK cells to CD11b^{m}CD27− NK cells is at least about 5× higher than the ratio of CD11b⁻CD27− NK cells to CD11b^{m}CD27− NK cells in NK cells obtained from a subject who does not have leukemia. In embodiments, the ratio of CD11b-CD27− NK cells to CD11b^{m}CD27− NK cells is at least about 6× higher than the ratio of CD11b⁻CD27− NK cells to CD11b^{m}CD27− NK cells in NK cells obtained from a subject who does not have leukemia. In embodiments, the ratio of CD11b⁻CD27− NK cells to CD11b^{m}CD27− NK cells is at least about 7× higher than the ratio of CD11b⁻CD27− NK cells to CD11b^{m}CD27− NK cells in NK cells obtained from a subject who does not have leukemia. In embodiments, the ratio of CD11b⁻CD27− NK cells to CD11b^{m}CD27− NK cells is at least about 8× higher than the ratio of CD11b⁻CD27− NK cells to CD11b^{m}CD27− NK cells in NK cells obtained from a subject who does not have leukemia. In embodiments, the ratio of CD11b⁻ CD27− NK cells to CD11b⁺CD27− NK cells is at least about 9× higher than the ratio in NK cells obtained from a subject who does not have leukemia. In embodiments, the ratio of CD11b⁻CD27− NK cells to CD11b⁺CD27− NK cells is at least about 10× higher than the ratio in NK cells obtained from a subject who does not have leukemia. In embodiments, the ratio of CD11b⁻CD27− NK cells to CD11b⁺CD27− NK cells is at least about 20× higher than the ratio in NK cells obtained from a subject who does not have leukemia. In embodiments, the ratio of CD11b⁻CD27− NK cells to CD11b⁺CD27− NK cells is at least about 30× higher than the ratio in NK cells obtained from a subject who does not have leukemia. In embodiments, the ratio of CD11b⁻CD27− NK cells to CD11b⁺CD27− NK cells is at least about 40× higher than the ratio in NK cells obtained from a subject who does not have leukemia. In embodiments, the ratio of CD11b⁻CD27− NK cells to CD11b⁺CD27− NK cells is at least about 50× higher than the ratio in NK cells obtained from a subject who does not have leukemia. In embodiments, the ratio of CD11b⁻CD27− NK cells to CD11b⁺CD27− NK cells is at least about 60× higher than the ratio in NK cells obtained from a subject who does not have leukemia. In embodiments, the ratio of CD11b⁻CD27− NK cells to CD11b⁺CD27− NK cells is at least about 70× higher than the ratio in NK cells obtained from a subject who does not have leukemia. In embodiments, the ratio of CD11b⁻CD27− NK cells to CD11b⁺CD27− NK cells is at least about 80× higher than the ratio in NK cells obtained from a subject who does not have leukemia. In embodiments, the ratio of CD11b⁻CD27− NK cells to CD11b⁺CD27− NK cells is at least about 90× higher than the ratio in NK cells obtained from a subject who does not have leukemia. In embodiments, the ratio of CD11b⁻CD27− NK cells to CD11b⁺CD27− NK cells is at least about 100× higher than the ratio in NK cells obtained from a subject who does not have leukemia. In embodiments, the ratio of CD11b⁻CD27− NK cells to CD11b⁺CD27− NK cells is at least about 150× higher than the ratio in NK cells obtained from a subject who does not have leukemia. In embodiments, the ratio of CD11b⁻CD27− NK cells to CD11b⁺CD27− NK cells is at least about 200× higher than the ratio in NK cells obtained from a subject who does not have leukemia.

In embodiments, an elevated ratio of CD11b⁻CD27− NK cells to CD11b⁺CD27− NK cells refers to a higher frequency of CD11b⁻CD27− NK cells to CD11b⁺CD27− NK cells relative to the frequency of CD11b⁻CD27− NK cells to CD11b⁺CD27− NK cells in a population of stage 5 NK cells (e.g. standard control). In embodiments, the population of population of stage 5 NK cells are obtained from a subject who does not have leukemia.

For the methods provided herein, in embodiments, the presence of CD69 is detected if the expression level of CD69 is elevated relative to a standard control. In embodiments, the standard control is the expression level of CD69 in an NK cell obtained from a subject who does not have leukemia. In embodiments, the standard control is the expression level of CD69 in a population of NK cells obtained from a subject who does not have leukemia. In embodiments, the standard control is the mean expression level of CD69 in the population of NK cells obtained from a subject who does not have leukemia. For example, the presence of CD69 may be detected if the expression level of CD69 in the population of NK cells is greater than the mean expression level of CD69 in a population of NK cells obtained from a subject who does not have leukemia. The mean expression level may be correlated to the mean fluorescent intensity when detected by cytometric methods. The mean expression level may be measured as mean mRNA transcript levels when detected by sequencing methods (e.g. single cell sequencing). In embodiments, the expression level of CD69 is undetectable in the standard control. In embodiments, the level of CD69 is undetectable in the standard control when using a cytometric method or a sequencing method (e.g. single cell sequencing). In embodiments, the presence of CD69 is detected if the expression level of CD69 is measureable by any method known in the art (e.g. cytometric methods, sequencing methods, antibody based methods, Western blot, Northern blot, etc.).

In embodiments, the presence of CD69 is detected if the expression level of CD69 is elevated relative to CD69 expression in a stage 5 NK cell (e.g. standard control). In embodiments, the presence of CD69 is detected if the expression level of CD69 is elevated relative to CD69 expression in a population of stage 5 NK cells (e.g. standard control) In embodiments, the presence of CD69 is detected if the expression level of CD69 is greater than the mean CD69 expression level in a population of stage 5 NK cells (e.g. standard control).

In embodiments, the population of NK cells has an increased frequency of NK cells that express CD69 relative to a standard control. In embodiments, the standard control is the frequency of NK cells that express CD69 in a population of NK cells obtained from a subject who does not have leukemia. In embodiments, the standard control is the frequency of NK cells expressing CD69 in a population of NK cells obtained from a subject who does not have leukemia. As described above, in embodiments, CD69 expression may refer to an expression level higher than the mean CD69 expression level in the population of NK cells obtained from a subject who does not have leukemia. In embodiments, the frequency of NK cells that express CD69 in the population of NK cells is elevated by at least 5% relative to the standard control. In embodiments, the frequency of NK cells that express CD69 in the population of NK cells is elevated by at least 10% relative to the standard control. In embodiments, the frequency of NK cells that express CD69 in the population of NK cells is elevated by at least 15% relative to the standard control. In embodiments, the frequency of NK cells that express CD69 in the population of NK cells is elevated by at least 20% relative to the standard control. In embodiments, the frequency of NK cells that express CD69 in the population of NK cells is elevated by at least 25% relative to the standard control. In embodiments, the frequency of NK cells that express CD69 in the population of NK cells is elevated by at least 30% relative to the standard control. In embodiments, the frequency of NK cells that express CD69 in the population of NK cells is elevated by at least 35% relative to the standard control. In embodiments, the frequency of NK cells that express CD69 in the population of NK cells is elevated by at least 40% relative to the standard control. In embodiments, the frequency of NK cells that express CD69 in the population of NK cells is elevated by at least 45% relative to the standard control. In embodiments, the frequency of NK cells that express CD69 in the population of NK cells is elevated by at least 50% relative to the standard control. In embodiments, the frequency of NK cells that express CD69 in the population of NK cells is elevated by at least 55% relative to the standard control. In embodiments, the frequency of NK cells that express CD69 in the population of NK cells is elevated by at least 60% relative to the standard control. In embodiments, the frequency of NK cells that express CD69 in the population of NK cells is elevated by at least 65% relative to the standard control. In embodiments, the frequency of NK cells that express CD69 in the population of NK cells is elevated by at least 75% relative to the standard control. In embodiments, the frequency of NK cells that express CD69 in the population of NK cells is elevated by at least 80% relative to the standard control. In embodiments, the frequency of NK cells that express CD69 in the population of NK cells is elevated by at least 85% relative to the standard control. In embodiments, the frequency of NK cells that express CD69 in the population of NK cells is elevated by at least 90% relative to the standard control. In embodiments, the frequency of NK cells that express CD69 in the population of NK cells is elevated by at least 95% relative to the standard control. In embodiments, the frequency of NK cells that express CD69 in the population of NK cells is elevated by at least 100% relative to the standard control.

In embodiments, the frequency of NK cells that express CD69 in the population of NK cells is elevated by at least 0.1× relative to the standard control. In embodiments, the frequency of NK cells that express CD69 in the population of NK cells is elevated by at least 0.5× relative to the standard control. In embodiments, the frequency of NK cells that express CD69 in the population of NK cells is elevated by at least 1× relative to the standard control. In embodiments, the frequency of NK cells that express CD69 in the population of NK cells is elevated by at least 2× relative to the standard control. In embodiments, the frequency of NK cells that express CD69 in the population of NK cells is elevated by at least 3× relative to the standard control. In embodiments, the frequency of NK cells that express CD69 in the population of NK cells is elevated by at least 4× relative to the standard control. In embodiments, the frequency of NK cells that express CD69 in the population of NK cells is elevated by at least 5× relative to the standard control. In embodiments, the frequency of NK cells that express CD69 in the population of NK cells is elevated by at least 6× relative to the standard control. In embodiments, the frequency of NK cells that express CD69 in the population of NK cells is elevated by at least 7× relative to the standard control. In embodiments, the frequency of NK cells that express CD69 in the population of NK cells is elevated by at least 8× relative to the standard control. In embodiments, the frequency of NK cells that express CD69 in the population of NK cells is elevated by at least 9× relative to the standard control. In embodiments, the frequency of NK cells that express CD69 in the population of NK cells is elevated by at least 10× relative to the standard control. In embodiments, the frequency of NK cells that express CD69 in the population of NK cells is elevated by at least 20× relative to the standard control. In embodiments, the frequency of NK cells that express CD69 in the population of NK cells is elevated by at least 30× relative to the standard control. In embodiments, the frequency of NK cells that express CD69 in the population of NK cells is elevated by at least 40× relative to the standard control. In embodiments, the frequency of NK cells that express CD69 in the population of NK cells is elevated by at least 50× relative to the standard control. In embodiments, the frequency of NK cells that express CD69 in the population of NK cells is elevated by at least 60× relative to the standard control. In embodiments, the frequency of NK cells that express CD69 in the population of NK cells is elevated by at least 70× relative to the standard control. In embodiments, the frequency of NK cells that express CD69 in the population of NK cells is elevated by at least 80× relative to the standard control. In embodiments, the frequency of NK cells that express CD69 in the population of NK cells is elevated by at least 90× relative to the standard control. In embodiments, the frequency of NK cells that express CD69 in the population of NK cells is elevated by at least 100× relative to the standard control. In embodiments, the frequency of NK cells that express CD69 in the population of NK cells is elevated by at least 150× relative to the standard control. In embodiments, the frequency of NK cells that express CD69 in the population of NK cells is elevated by at least 200× relative to the standard control.

For the methods provided herein, in embodiments, the cytokine is Interferon gamma (IFN-γ), Tumor necrosis factor (TNF), Granulocyte-macrophage colony-stimulating factor (GM-CSF), Macrophage inflammatory protein 1 beta (MIP-10), Interleukin-2 (IL-2), or combinations thereof. In embodiments, the cytokine is IFN-γ, TNF, GM-CSF, or combinations thereof. In embodiments, the cytokine is IFN-γ. In embodiments, the cytokine is TNF. In embodiments, the cytokine is GM-CSF. In embodiments, the cytokine is MIP-1β. In embodiments, the cytokine is IL-2.

In embodiments, the presence of a cytokine (e.g. IFN-γ, TNF, GM-CSF, etc.) is detected if the expression level of the cytokine is elevated relative to a standard control. In embodiments, the standard control is the expression level of the cytokine in an NK cell obtained from a subject who does not have leukemia. In embodiments, the standard control is the expression level of the cytokine of in a population of NK cells obtained from a subject who does not have leukemia. In embodiments, the standard control is the mean expression level of the cytokine in the population of NK cells obtained from a subject who does not have leukemia. In embodiments, the standard control is mean expression level of the cytokine in a population of resting NK cells. The mean expression level may correlate to mean fluorescent intensity when detected by cytometric methods. The mean expression level may be measured as mean mRNA transcript levels when detected by sequencing methods (e.g. single cell sequencing). In embodiments, the expression level of the cytokine is undetectable in the standard control. In embodiments, the presence of the cytokine (e.g. IFN-γ, TNF, GM-CSF, etc.) is detected if the cytokine is detectable by any method known in the art (e.g. cytometric methods, sequencing methods, antibody based methods, Western Blot, Northern Blot, etc.).

In embodiments, the presence of a cytokine is detected if the expression level of the cytokine is elevated relative to the cytokine expression level in a stage 5 NK cell (e.g. standard control). In embodiments, the presence of a cytokine is detected if the expression level of the cytokine is elevated relative to the cytokine expression level in a population of stage 5 NK cells (e.g. standard control) In embodiments, the presence of the cytokine is detected if the expression level of the cytokine is greater than the mean cytokine expression level of the cytokine in a population of stage 5 NK cells (e.g. standard control).

In embodiments, the population of NK cells has an increased frequency of NK cells that express the cytokine (e.g. IFN-γ, TNF, GM-CSF, etc.) relative to a standard control. In embodiments, the standard control is the frequency of NK that express the cytokine in a population of NK cells obtained from a subject who does not have leukemia. In embodiments, the standard control is the frequency of NK cells expressing the cytokine in a population of NK cells obtained from a subject who does not have leukemia. As described above, in embodiments, cytokine expression refers to an expression level higher than the mean expression level in the population of NK cells obtained from the subject who does not have leukemia. In embodiments, the frequency of NK cells that express the cytokine in the population of NK cells is elevated by at least 5% relative to the standard control. In embodiments, the frequency of NK cells that express the cytokine in the population of NK cells is elevated by at least 10% relative to the standard control. In embodiments, the frequency of NK cells that express the cytokine in the population of NK cells is elevated by at least 15% relative to the standard control. In embodiments, the frequency of NK cells that express the cytokine in the population of NK cells is elevated by at least 20% relative to the standard control. In embodiments, the frequency of NK cells that express the cytokine in the population of NK cells is elevated by at least 25% relative to the standard control. In embodiments, the frequency of NK cells that express the cytokine in the population of NK cells is elevated by at least 30% relative to the standard control. In embodiments, the frequency of NK cells that express the cytokine in the population of NK cells is elevated by at least 35% relative to the standard control. In embodiments, the frequency of NK cells that express the cytokine in the population of NK cells is elevated by at least 40% relative to the standard control. In embodiments, the frequency of NK cells that express the cytokine in the population of NK cells is elevated by at least 45% relative to the standard control. In embodiments, the frequency of NK cells that express the cytokine in the population of NK cells is elevated by at least 50% relative to the standard control. In embodiments, the frequency of NK cells that express the cytokine in the population of NK cells is elevated by at least 55% relative to the standard control. In embodiments, the frequency of NK cells that express the cytokine in the population of NK cells is elevated by at least 60% relative to the standard control. In embodiments, the frequency of NK cells that express the cytokine in the population of NK cells is elevated by at least 65% relative to the standard control. In embodiments, the frequency of NK cells that express the cytokine in the population of NK cells is elevated by at least 75% relative to the standard control. In embodiments, the frequency of NK cells that express the cytokine in the population of NK cells is elevated by at least 80% relative to the standard control. In embodiments, the frequency of NK cells that express the cytokine in the population of NK cells is elevated by at least 85% relative to the standard control. In embodiments, the frequency of NK cells that express the cytokine in the population of NK cells is elevated by at least 90% relative to the standard control. In embodiments, the frequency of NK cells that express the cytokine in the population of NK cells is elevated by at least 95% relative to the standard control. In embodiments, the frequency of NK cells that express the cytokine in the population of NK cells is elevated by at least 100% relative to the standard control.

In embodiments, the frequency of NK cells that express the cytokine in the population of NK cells is elevated by at least 0.1× relative to the standard control. In embodiments, the frequency of NK cells that express the cytokine in the population of NK cells is elevated by at least 0.5× relative to the standard control. In embodiments, the frequency of NK cells that express the cytokine in the population of NK cells is elevated by at least 1× relative to the standard control. In embodiments, the frequency of NK cells that express the cytokine in the population of NK cells is elevated by at least 2× relative to the standard control. In embodiments, the frequency of NK cells that express the cytokine in the population of NK cells is elevated by at least 3× relative to the standard control. In embodiments, the frequency of NK cells that express the cytokine in the population of NK cells is elevated by at least 4× relative to the standard control. In embodiments, the frequency of NK cells that express the cytokine in the population of NK cells is elevated by at least 5× relative to the standard control. In embodiments, the frequency of NK cells that express the cytokine in the population of NK cells is elevated by at least 6× relative to the standard control. In embodiments, the frequency of NK cells that express the cytokine in the population of NK cells is elevated by at least 7× relative to the standard control. In embodiments, the frequency of NK cells that express the cytokine in the population of NK cells is elevated by at least 8× relative to the standard control. In embodiments, the frequency of NK cells that express the cytokine in the population of NK cells is elevated by at least 9× relative to the standard control. In embodiments, the frequency of NK cells that express the cytokine in the population of NK cells is elevated by at least 10× relative to the standard control. In embodiments, the frequency of NK cells that express the cytokine in the population of NK cells is elevated by at least 20× relative to the standard control. In embodiments, the frequency of NK cells that express the cytokine in the population of NK cells is elevated by at least 30× relative to the standard control. In embodiments, the frequency of NK cells that express the cytokine in the population of NK cells is elevated by at least 40× relative to the standard control. In embodiments, the frequency of NK cells that express the cytokine in the population of NK cells is elevated by at least 50× relative to the standard control. In embodiments, the frequency of NK cells that express the cytokine in the population of NK cells is elevated by at least 60× relative to the standard control. In embodiments, the frequency of NK cells that express the cytokine in the population of NK cells is elevated by at least 70× relative to the standard control. In embodiments, the frequency of NK cells that express the cytokine in the population of NK cells is elevated by at least 80× relative to the standard control. In embodiments, the frequency of NK cells that express the cytokine in the population of NK cells is elevated by at least 90× relative to the standard control. In embodiments, the frequency of NK cells that express the cytokine in the population of NK cells is elevated by at least 100× relative to the standard control. In embodiments, the frequency of NK cells that express the cytokine in the population of NK cells is elevated by at least 150× relative to the standard control. In embodiments, the frequency of NK cells that express the cytokine in the population of NK cells is elevated by at least 200× relative to the standard control.

In embodiments, detecting calcium (Ca²⁺) mobilization includes detection of Ca²⁺ in the population of NK cells. In embodiments, detection of Ca²⁺ includes contacting the NK cells with a compound that binds Ca²⁺ (e.g. a calcium indicator) and detecting binding of the compound to Ca²⁺ (e.g. by a cytometric method). For example, the compound (e.g. a calcium indicator) may have an first emission peak prior to binding Ca²⁺ and a second emission peak when bound to Ca²⁺, thereby allowing detection of calcium mobilization by detecting the emission wavelength shift. In embodiments, the compound is a calcium indicator. “Calcium indicator” is used in accordance to its plain ordinary meaning in the arts and refers to a compound that undergoes a change in fluorescent intensity or an emission/excitation wavelength shift upon binding calcium. Calcium indicators include Indo-1, Indol AM, Fura-2, or Fura-2 AM. In embodiments, the calcium indicator is Indo-1. In embodiments, detection of calcium includes detecting elevated expression levels of genes in the calcium signaling pathway relative to a standard control. Expression levels of genes may be detected by methods known in the art including antibody based methods, cytometric methods, and sequencing methods (e.g. single cell sequencing, gene set enrichment analysis, etc.).

In embodiments, Ca²⁺ mobilization is detected if the level of Ca²⁺ (e.g. as measured by a calcium indicator) is elevated relative to a standard control. In embodiments, the standard control is level of Ca²⁺ in an NK cell obtained from a subject who does not have leukemia. In embodiments, the standard control is level of Ca²⁺ in a population of NK cells obtained from a subject who does not have leukemia. For example, Ca²⁺ mobilization is detected if the area under a fluorescence emission curve of a calcium indicator of is greater than the area of a fluorescence emission curve of a calcium indicator in a population of cells obtained from a subject who does not have leukemia (standard control). In embodiments, the standard control is a stage 5 NK cell. In embodiments, the standard control is a population of stage 5 NK cells.

In embodiments, the level of Ca²⁺ (e.g. calcium mobilization) is at least 5% higher than the standard control (e.g. population of cells obtained from a subject who does not have leukemia). In embodiments, the level of Ca²⁺ (e.g. calcium mobilization) is at least 10% higher than the standard control (e.g. population of cells obtained from a subject who does not have leukemia). In embodiments, the level of Ca²⁺ (e.g. calcium mobilization) is at least 15% higher than the standard control (e.g. population of cells obtained from a subject who does not have leukemia). In embodiments, the level of Ca²⁺ (e.g. calcium mobilization) is at least 20% higher than the standard control (e.g. population of cells obtained from a subject who does not have leukemia). In embodiments, the level of Ca²⁺ (e.g. calcium mobilization) is at least 25% higher than the standard control (e.g. population of cells obtained from a subject who does not have leukemia). In embodiments, the level of Ca²⁺ (e.g. calcium mobilization) is at least 30% higher than the standard control (e.g. population of cells obtained from a subject who does not have leukemia). In embodiments, the level of Ca²⁺ (e.g. calcium mobilization) is at least 35% higher than the standard control (e.g. population of cells obtained from a subject who does not have leukemia). In embodiments, the level of Ca²⁺ (e.g. calcium mobilization) is at least 40% higher than the standard control (e.g. population of cells obtained from a subject who does not have leukemia). In embodiments, the level of Ca²⁺ (e.g. calcium mobilization) is at least 45% higher than the standard control (e.g. population of cells obtained from a subject who does not have leukemia). In embodiments, the level of Ca²⁺ (e.g. calcium mobilization) is at least 50% higher than the standard control (e.g. population of cells obtained from a subject who does not have leukemia). In embodiments, the level of Ca²⁺ (e.g. calcium mobilization) is at least 55% higher than the standard control (e.g. population of cells obtained from a subject who does not have leukemia). In embodiments, the level of Ca²⁺ (e.g. calcium mobilization) is at least 60% higher than the standard control (e.g. population of cells obtained from a subject who does not have leukemia). In embodiments, the level of Ca²⁺ (e.g. calcium mobilization) is at least 65% higher than the standard control (e.g. population of cells obtained from a subject who does not have leukemia). In embodiments, the level of Ca²⁺ (e.g. calcium mobilization) is at least 70% higher than the standard control (e.g. population of cells obtained from a subject who does not have leukemia). In embodiments, the level of Ca²⁺ (e.g. calcium mobilization) is at least 75% higher than the standard control (e.g. population of cells obtained from a subject who does not have leukemia). In embodiments, the level of Ca²⁺ (e.g. calcium mobilization) is at least 80% higher than the standard control (e.g. population of cells obtained from a subject who does not have leukemia). In embodiments, the level of Ca²⁺ (e.g. calcium mobilization) is at least 85% higher than the standard control (e.g. population of cells obtained from a subject who does not have leukemia). In embodiments, the level of Ca²⁺ (e.g. calcium mobilization) is at least 90% higher than the standard control (e.g. population of cells obtained from a subject who does not have leukemia). In embodiments, the level of Ca²⁺ (e.g. calcium mobilization) is at least 95% higher than the standard control (e.g. population of cells obtained from a subject who does not have leukemia). In embodiments, the level of Ca²⁺ (e.g. calcium mobilization) is at least 100% higher than the standard control (e.g. population of cells obtained from a subject who does not have leukemia).

In embodiments, the level of Ca²⁺ (e.g. calcium mobilization) is at least 0.1× higher than the standard control (e.g. population of cells obtained from a subject who does not have leukemia). In embodiments, the level of Ca²⁺ (e.g. calcium mobilization) is at least 0.5× higher than the standard control (e.g. population of cells obtained from a subject who does not have leukemia). In embodiments, the level of Ca²⁺ (e.g. calcium mobilization) is at least 1× higher than the standard control (e.g. population of cells obtained from a subject who does not have leukemia). In embodiments, the level of Ca²⁺ (e.g. calcium mobilization) is at least 2× higher than the standard control (e.g. population of cells obtained from a subject who does not have leukemia). In embodiments, the level of Ca²⁺ (e.g. calcium mobilization) is at least 3× higher than the standard control (e.g. population of cells obtained from a subject who does not have leukemia). In embodiments, the level of Ca²⁺ (e.g. calcium mobilization) is at least 4× higher than the standard control (e.g. population of cells obtained from a subject who does not have leukemia). In embodiments, the level of Ca²⁺ (e.g. calcium mobilization) is at least 5× higher than the standard control (e.g. population of cells obtained from a subject who does not have leukemia). In embodiments, the level of Ca²⁺ (e.g. calcium mobilization) is at least 6× higher than the standard control (e.g. population of cells obtained from a subject who does not have leukemia). In embodiments, the level of Ca²⁺ (e.g. calcium mobilization) is at least 7× higher than the standard control (e.g. population of cells obtained from a subject who does not have leukemia). In embodiments, the level of Ca²⁺ (e.g. calcium mobilization) is at least 8× higher than the standard control (e.g. population of cells obtained from a subject who does not have leukemia). In embodiments, the level of Ca²⁺ (e.g. calcium mobilization) is at least 9× higher than the standard control (e.g. population of cells obtained from a subject who does not have leukemia). In embodiments, the level of Ca²⁺ (e.g. calcium mobilization) is at least 10× higher than the standard control (e.g. population of cells obtained from a subject who does not have leukemia). In embodiments, the level of Ca²⁺ (e.g. calcium mobilization) is at least 20× higher than the standard control (e.g. population of cells obtained from a subject who does not have leukemia). In embodiments, the level of Ca²⁺ (e.g. calcium mobilization) is at least 30× higher than the standard control (e.g. population of cells obtained from a subject who does not have leukemia). In embodiments, the level of Ca²⁺ (e.g. calcium mobilization) is at least 40× higher than the standard control (e.g. population of cells obtained from a subject who does not have leukemia). In embodiments, the level of Ca²⁺ (e.g. calcium mobilization) is at least 50× higher than the standard control (e.g. population of cells obtained from a subject who does not have leukemia). In embodiments, the level of Ca²⁺ (e.g. calcium mobilization) is at least 60× higher than the standard control (e.g. population of cells obtained from a subject who does not have leukemia). In embodiments, the level of Ca²⁺ (e.g. calcium mobilization) is at least 70× higher than the standard control (e.g. population of cells obtained from a subject who does not have leukemia). In embodiments, the level of Ca²⁺ (e.g. calcium mobilization) is at least 80× higher than the standard control (e.g. population of cells obtained from a subject who does not have leukemia). In embodiments, the level of Ca²⁺ (e.g. calcium mobilization) is at least 90× higher than the standard control (e.g. population of cells obtained from a subject who does not have leukemia). In embodiments, the level of Ca²⁺ (e.g. calcium mobilization) is at least 100× higher than the standard control (e.g. population of cells obtained from a subject who does not have leukemia). In embodiments, the level of Ca²⁺ (e.g. calcium mobilization) is at least 150× higher than the standard control (e.g. population of cells obtained from a subject who does not have leukemia). In embodiments, the level of Ca²⁺ (e.g. calcium mobilization) is at least 200× higher than the standard control (e.g. population of cells obtained from a subject who does not have leukemia).

In embodiments, the method further includes detecting the absence of DNAX Accessory Molecule-1 (DNAM 1), Killer cell immunoglobulin-like receptor 2DL1 (KIR2DL1), CD57, Siglec-7, or combinations thereof in the population of NK cells. In embodiments, the method further includes detecting the absence of DNAM 1 in the population of NK cells. In embodiments, the method further includes detecting the absence KIR2DL1 in the population of NK cells. In embodiments, the method further includes detecting the absence of CD57 in the population of NK cells in the population of NK cells. In embodiments, the method further includes detecting the absence of Siglec-7 in the population of NK cells. In embodiments, absence of DNAM 1, KIR2DL1, CD57, or Siglec-7 indicates that the expression level of DNAM 1, KIR2DL1, CD57, or Siglec-7 expression level is undetectable (e.g. by cytometric methods, sequencing methods, antibody based methods, etc.).

In embodiments, absence of DNAM 1, KIR2DL1, CD57, or Siglec-7 is an expression level at least 50% lower than the expression level of DNAM 1, KIR2DL1, CD57, or Siglec-7 in a standard control. In embodiments, the standard control is an NK cell obtained from a subject who does not have leukemia. In embodiments, the standard control is the mean expression level of DNAM 1, KIR2DL1, CD57, or Siglec-7 in a population of NK cells obtained from a subject who does not have leukemia. For example, absence of DNAM 1 may be an expression level lower than the mean expression of level of DNAM 1 in a population of NK cells obtained from a subject who does not have leukemia. For example, absence of KIR2DL1 is an expression level at least 50% lower than the mean expression of level of KIR2DL1 in a population of NK cells obtained from a subject who does not have leukemia. In another example, absence of CD57 is an expression level at least 50% lower than the mean expression of level of CD57 in a population of NK cells obtained from a subject who does not have leukemia. In another example, absence of Siglec-7 may be an expression level at least 50% lower than the mean expression of level of Siglec-7 in a population of NK cells obtained from a subject who does not have leukemia.

In embodiments, the standard control is a stage 5 NK cell. In embodiments, the standard control is a population of stage 5 NK cells. In embodiments, the standard control is the mean expression level of DNAM 1, KIR2DL1, CD57, or Siglec-7 in a population of stage 5 NK cells. For example, absence of DNAM 1, KIR2DL1, CD57, or Siglec-7 may be an expression level lower than the mean expression of level of DNAM 1, KIR2DL1, CD57, or Siglec-7 in a population of stage 5 NK cells.

In embodiments, absence of DNAM 1, KIR2DL1, CD57, or Siglec-7 is an expression level at least 60% lower than the expression level in a standard control. In embodiments, absence of DNAM 1, KIR2DL1, CD57, or Siglec-7 is an expression level at least 70% lower than the expression level in a standard control. In embodiments, absence of DNAM 1, KIR2DL1, CD57, or Siglec-7 is an expression level at least 80% lower than the expression level in a standard control. In embodiments, absence of DNAM 1, KIR2DL1, CD57, or Siglec-7 is an expression level at least 85% lower than the expression level in a standard control. In embodiments, absence of DNAM 1, KIR2DL1, CD57, or Siglec-7 is an expression level at least 90% lower than the expression level in a standard control. In embodiments, absence of DNAM 1, KIR2DL1, CD57, or Siglec-7 is an expression level at least 95% lower than the expression level in a standard control. In embodiments, absence of DNAM 1, KIR2DL1, CD57, or Siglec-7 is an expression level at least 98% lower than the expression level in a standard control. In embodiments, absence of DNAM 1, KIR2DL1, CD57, or Siglec-7 is an expression level at least 99% lower than the expression level in a standard control. In embodiments, absence of DNAM 1, KIR2DL1, CD57, or Siglec-7 is an expression level 100% lower than the expression level in a standard control. In embodiments, absence of DNAM 1, KIR2DL1, CD57, or Siglec-7 indicates that DNAM 1, KIR2DL1, CD57, or Siglec-7 is undetectable by methods known in the art (e.g. cytometric methods, sequencing methods, etc.). Absence of DNAM 1, KIR2DL1, CD57, or Siglec-7 may refer to absence of DNAM 1, KIR2DL1, CD57, or Siglec-7 mRNA transcript. Absence of DNAM 1, KIR2DL1, CD57, or Siglec-7 may refer to absence of DNAM 1, KIR2DL1, CD57, or Siglec-7 protein.

In embodiments, absence of DNAM 1, KIR2DL1, CD57, or Siglec-7 is an expression level at least 0.5× lower than the expression level in a standard control. In embodiments, absence of DNAM 1, KIR2DL1, CD57, or Siglec-7 is an expression level at least 1× lower than the expression level in a standard control. In embodiments, absence of DNAM 1, KIR2DL1, CD57, or Siglec-7 is an expression level at least 2× lower than the expression level in a standard control. In embodiments, absence of DNAM 1, KIR2DL1, CD57, or Siglec-7 is an expression level at least 3× lower than the expression level in a standard control. In embodiments, absence of DNAM 1, KIR2DL1, CD57, or Siglec-7 is an expression level at least 4× lower than the expression level in a standard control. In embodiments, absence of DNAM 1, KIR2DL1, CD57, or Siglec-7 is an expression level at least 5× lower than the expression level in a standard control. In embodiments, absence of DNAM 1, KIR2DL1, CD57, or Siglec-7 is an expression level at least 6× lower than the expression level in a standard control. In embodiments, absence of DNAM 1, KIR2DL1, CD57, or Siglec-7 is an expression level at least 7× lower than the expression level in a standard control. In embodiments, absence of DNAM 1, KIR2DL1, CD57, or Siglec-7 is an expression level at least 8× lower than the expression level in a standard control. In embodiments, absence of DNAM 1, KIR2DL1, CD57, or Siglec-7 is an expression level at least 9× lower than the expression level in a standard control. In embodiments, absence of DNAM 1, KIR2DL1, CD57, or Siglec-7 is an expression level at least 10× lower than the expression level in a standard control. In embodiments, absence of DNAM 1, KIR2DL1, CD57, or Siglec-7 is an expression level at least 15× lower than the expression level in a standard control. In embodiments, absence of DNAM 1, KIR2DL1, CD57, or Siglec-7 is an expression level at least 20× lower than the expression level in a standard control. In embodiments, absence of DNAM 1, KIR2DL1, CD57, or Siglec-7 is an expression level at least 30× lower than the expression level in a standard control. In embodiments, absence of DNAM 1, KIR2DL1, CD57, or Siglec-7 is an expression level at least 40× lower than the expression level in a standard control. In embodiments, absence of DNAM 1, KIR2DL1, CD57, or Siglec-7 is an expression level at least 50× lower than the expression level in a standard control.

Despite their decreased capability in killing cancer cells, Applicants have demonstrated herein that dysfunctional NK cells may secrete cytotoxic granules. In fact, dysfunctional ALL cells have been shown secrete more cytotoxic granules than their healthy counterparts. Excess degranuation may be indicative that the dysfunctional NK cells are hyperactivated and exhausted, although as described above, the cytotoxic granules have decreased ability to kill cancer cells relative to granules secreted from subjects who do not have leukemia. Thus, in embodiments, the method further includes detecting the presence of cytotoxic granules in the population of NK cells. In embodiments, detecting the presence of cytotoxic granules includes detecting the presence of perforin (PRF) or granzyme B (GZMB) in the population of NK cells. In embodiments, detecting the presence of cytotoxic granules includes detecting the presence of PRF in the population of NK cells. In embodiments, detecting the presence of cytotoxic granules includes detecting the presence of GZMB in the population of NK cells.

In embodiments, the presence of PRF (e.g. PRF expression) is detected if the expression level of PRF is elevated relative to a standard control. In embodiments, the standard control is the expression level of PRF in an NK cell obtained from a subject who does not have leukemia. In embodiments, the standard control is the mean expression level of PRF in a population of NK cells obtained from a subject who does not have leukemia. The mean expression level may correlate to the mean fluorescent intensity when detected by cytometric methods. The mean expression level may be measured as mean mRNA transcript levels when detected by sequencing methods (e.g. single cell sequencing). In embodiments, the expression level of PRF is undetectable in the standard control. In embodiments, the presence of PRF is detected if the expression level of PRF is measureable by any method known in the art (e.g. cytometric methods, sequencing methods, antibody based methods, etc.).

In embodiments, the presence of PRF is detected if the expression level of PRF is elevated relative to PRF expression in a stage 5 NK cell (e.g. standard control). In embodiments, the presence of PRF is detected if the expression level of PRF is elevated relative to PRF expression in a population of stage 5 NK cells (e.g. standard control) In embodiments, the presence of PRF is detected if the expression level of PRF is greater than the mean PRF expression level in a population of stage 5 NK cells (e.g. standard control).

In embodiments, the population of NK cells has an increased frequency of NK cells that express PRF relative to a standard control. In embodiments, the standard control is the frequency of NK cells that express PRF in a population of NK cells obtained from a subject who does not have leukemia. In embodiments, the standard control is the frequency of NK cells expressing PRF in a population of NK cells obtained from a subject who does not have leukemia, wherein PRF expression refers to an expression level higher than the mean expression level of the population of cells obtained from the subject who does not have leukemia. In embodiments, the frequency of NK cells that express PRF in the population of NK cells is elevated by at least 5% relative to the standard control. In embodiments, the frequency of NK cells that express PRF in the population of NK cells is elevated by at least 10% relative to the standard control. In embodiments, the frequency of NK cells that express PRF in the population of NK cells is elevated by at least 15% relative to the standard control. In embodiments, the frequency of NK cells that express PRF in the population of NK cells is elevated by at least 20% relative to the standard control. In embodiments, the frequency of NK cells that express PRF in the population of NK cells is elevated by at least 25% relative to the standard control. In embodiments, the frequency of NK cells that express PRF in the population of NK cells is elevated by at least 30% relative to the standard control. In embodiments, the frequency of NK cells that express PRF in the population of NK cells is elevated by at least 35% relative to the standard control. In embodiments, the frequency of NK cells that express PRF in the population of NK cells is elevated by at least 40% relative to the standard control. In embodiments, the frequency of NK cells that express PRF in the population of NK cells is elevated by at least 45% relative to the standard control. In embodiments, the frequency of NK cells that express PRF in the population of NK cells is elevated by at least 50% relative to the standard control. In embodiments, the frequency of NK cells that express PRF in the population of NK cells is elevated by at least 55% relative to the standard control. In embodiments, the frequency of NK cells that express PRF in the population of NK cells is elevated by at least 60% relative to the standard control. In embodiments, the frequency of NK cells that express PRF in the population of NK cells is elevated by at least 65% relative to the standard control. In embodiments, the frequency of NK cells that express PRF in the population of NK cells is elevated by at least 75% relative to the standard control. In embodiments, the frequency of NK cells that express PRF in the population of NK cells is elevated by at least 80% relative to the standard control. In embodiments, the frequency of NK cells that express PRF in the population of NK cells is elevated by at least 85% relative to the standard control. In embodiments, the frequency of NK cells that express PRF in the population of NK cells is elevated by at least 90% relative to the standard control. In embodiments, the frequency of NK cells that express PRF in the population of NK cells is elevated by at least 95% relative to the standard control. In embodiments, the frequency of NK cells that express PRF in the population of NK cells is elevated by at least 100% relative to the standard control.

In embodiments, the frequency of NK cells that express PRF in the population of NK cells is elevated by at least 0.1× relative to the standard control. In embodiments, the frequency of NK cells that express PRF in the population of NK cells is elevated by at least 0.5× relative to the standard control. In embodiments, the frequency of NK cells that express PRF in the population of NK cells is elevated by at least 1× relative to the standard control. In embodiments, the frequency of NK cells that express PRF in the population of NK cells is elevated by at least 2× relative to the standard control. In embodiments, the frequency of NK cells that express PRF in the population of NK cells is elevated by at least 3× relative to the standard control. In embodiments, the frequency of NK cells that express PRF in the population of NK cells is elevated by at least 4× relative to the standard control. In embodiments, the frequency of NK cells that express PRF in the population of NK cells is elevated by at least 5× relative to the standard control. In embodiments, the frequency of NK cells that express PRF in the population of NK cells is elevated by at least 6× relative to the standard control. In embodiments, the frequency of NK cells that express PRF in the population of NK cells is elevated by at least 7× relative to the standard control. In embodiments, the frequency of NK cells that express PRF in the population of NK cells is elevated by at least 8× relative to the standard control. In embodiments, the frequency of NK cells that express PRF in the population of NK cells is elevated by at least 9× relative to the standard control. In embodiments, the frequency of NK cells that express PRF in the population of NK cells is elevated by at least 10× relative to the standard control. In embodiments, the frequency of NK cells that express PRF in the population of NK cells is elevated by at least 20× relative to the standard control. In embodiments, the frequency of NK cells that express PRF in the population of NK cells is elevated by at least 30× relative to the standard control. In embodiments, the frequency of NK cells that express PRF in the population of NK cells is elevated by at least 40× relative to the standard control. In embodiments, the frequency of NK cells that express PRF in the population of NK cells is elevated by at least 50× relative to the standard control. In embodiments, the frequency of NK cells that express PRF in the population of NK cells is elevated by at least 100× relative to the standard control. In embodiments, the frequency of NK cells that express PRF in the population of NK cells is elevated by at least 150× relative to the standard control. In embodiments, the frequency of NK cells that express PRF in the population of NK cells is elevated by at least 200× relative to the standard control.

In embodiments, the presence of GZMB (e.g. GZMB expression) is detected if the expression level of GZMB is elevated relative to a standard control. In embodiments, the standard control is the expression level of GZMB of an NK cell obtained from a subject who does not have leukemia. In embodiments, the standard control is the mean expression level of GZMB in a population of NK cells obtained from a subject who does not have leukemia. The mean expression level may correlate to mean fluorescent intensity when detected by cytometric methods. The mean expression level may be measured as mean mRNA transcript levels when detected by sequencing methods (e.g. single cell sequencing). In embodiments, the expression level of GZMB is undetectable in the standard control. In embodiments, the presence of GZMB is detected if the expression level of GZMB is measureable by any method known in the art (e.g. cytometric methods, sequencing methods, antibody based methods, etc.).

In embodiments, the presence of GZMB is detected if the expression level of GZMB is elevated relative to GZMB expression in a stage 5 NK cell (e.g. standard control). In embodiments, the presence of PRF is detected if the expression level of GZMB is elevated relative to GZMB expression in a population of stage 5 NK cells (e.g. standard control) In embodiments, the presence of GZMB is detected if the expression level of GZMB is greater than the mean GZMB expression level in a population of stage 5 NK cells (e.g. standard control).

In embodiments, the population of NK cells has an increased frequency of NK cells that express GZMB relative to a standard control. In embodiments, the standard control is the frequency of NK cells that express GZMB in a population of NK cells obtained from a subject who does not have leukemia. In embodiments, the standard control is the frequency of NK cells expressing GZMB in a population of NK cells obtained from a subject who does not have leukemia. As described above, in embodiments, GZMB expression may refer to an expression level higher than the mean expression level of the population of cells obtained from the subject who does not have leukemia. In embodiments, the frequency of NK cells that express GZMB in the population of NK cells is elevated by at least 5% relative to the standard control. In embodiments, the frequency of NK cells that express GZMB in the population of NK cells is elevated by at least 10% relative to the standard control. In embodiments, the frequency of NK cells that express GZMB in the population of NK cells is elevated by at least 15% relative to the standard control. In embodiments, the frequency of NK cells that express GZMB in the population of NK cells is elevated by at least 20% relative to the standard control. In embodiments, the frequency of NK cells that express GZMB in the population of NK cells is elevated by at least 25% relative to the standard control. In embodiments, the frequency of NK cells that express GZMB in the population of NK cells is elevated by at least 30% relative to the standard control. In embodiments, the frequency of NK cells that express GZMB in the population of NK cells is elevated by at least 35% relative to the standard control. In embodiments, the frequency of NK cells that express GZMB in the population of NK cells is elevated by at least 40% relative to the standard control. In embodiments, the frequency of NK cells that express GZMB in the population of NK cells is elevated by at least 45% relative to the standard control. In embodiments, the frequency of NK cells that express GZMB in the population of NK cells is elevated by at least 50% relative to the standard control. In embodiments, the frequency of NK cells that express GZMB in the population of NK cells is elevated by at least 55% relative to the standard control. In embodiments, the frequency of NK cells that express GZMB in the population of NK cells is elevated by at least 60% relative to the standard control. In embodiments, the frequency of NK cells that express GZMB in the population of NK cells is elevated by at least 65% relative to the standard control. In embodiments, the frequency of NK cells that express GZMB in the population of NK cells is elevated by at least 75% relative to the standard control. In embodiments, the frequency of NK cells that express GZMB in the population of NK cells is elevated by at least 80% relative to the standard control. In embodiments, the frequency of NK cells that express GZMB in the population of NK cells is elevated by at least 85% relative to the standard control. In embodiments, the frequency of NK cells that express GZMB in the population of NK cells is elevated by at least 90% relative to the standard control. In embodiments, the frequency of NK cells that express GZMB in the population of NK cells is elevated by at least 95% relative to the standard control. In embodiments, the frequency of NK cells that express GZMB in the population of NK cells is elevated by at least 100% relative to the standard control.

In embodiments, the frequency of NK cells that express GZMB in the population of NK cells is elevated by at least 0.1× relative to the standard control. In embodiments, the frequency of NK cells that express GZMB in the population of NK cells is elevated by at least 0.5× relative to the standard control. In embodiments, the frequency of NK cells that express GZMB in the population of NK cells is elevated by at least 1× relative to the standard control. In embodiments, the frequency of NK cells that express GZMB in the population of NK cells is elevated by at least 2× relative to the standard control. In embodiments, the frequency of NK cells that express GZMB in the population of NK cells is elevated by at least 3× relative to the standard control. In embodiments, the frequency of NK cells that express GZMB in the population of NK cells is elevated by at least 4× relative to the standard control. In embodiments, the frequency of NK cells that express GZMB in the population of NK cells is elevated by at least 5× relative to the standard control. In embodiments, the frequency of NK cells that express GZMB in the population of NK cells is elevated by at least 6× relative to the standard control. In embodiments, the frequency of NK cells that express GZMB in the population of NK cells is elevated by at least 7× relative to the standard control. In embodiments, the frequency of NK cells that express GZMB in the population of NK cells is elevated by at least 8× relative to the standard control. In embodiments, the frequency of NK cells that express GZMB in the population of NK cells is elevated by at least 9× relative to the standard control. In embodiments, the frequency of NK cells that express GZMB in the population of NK cells is elevated by at least 10× relative to the standard control. In embodiments, the frequency of NK cells that express GZMB in the population of NK cells is elevated by at least 20× relative to the standard control. In embodiments, the frequency of NK cells that express GZMB in the population of NK cells is elevated by at least 30× relative to the standard control. In embodiments, the frequency of NK cells that express GZMB in the population of NK cells is elevated by at least 40× relative to the standard control. In embodiments, the frequency of NK cells that express GZMB in the population of NK cells is elevated by at least 50× relative to the standard control. In embodiments, the frequency of NK cells that express GZMB in the population of NK cells is elevated by at least 100× relative to the standard control. In embodiments, the frequency of NK cells that express GZMB in the population of NK cells is elevated by at least 150× relative to the standard control. In embodiments, the frequency of NK cells that express GZMB in the population of NK cells is elevated by at least 200× relative to the standard control.

As described above, Applicant has discovered that dysfunctional NK cells may be hyperactivated, although they have decreased cell killing ability relative to their healthy counterparts (e.g. NK cells obtained from non-leukemic subjects). For example, Applicant has shown that immature NK cells from leukemia patients fail to kill lymphoblasts although they may express activation markers. Thus, dysfunctional NK cells may be identified by detecting the presence of an activation marker. “Activation marker” as used herein refers to a molecule produced by an NK cell that promotes NK cell proliferation, NK cell maturation, expression of signaling molecules including cytokines and their cognate receptors, or generation of cytotoxic granules. Expression of an activation marker typically increases cancer cell killing; however in a dysfunctional NK cell an activation marker may indicate that the NK cell is exhausted. For example, an exhausted NK cell may have poor cell killing function and higher expression of inhibitory molecules. Thus, in embodiments, the method further includes detecting the presence of one or more activation markers in the population of NK cells. In embodiments, the activation marker is CD107a.

In embodiments, the presence of CD107a is detected if the expression level of CD107a is elevated relative to a standard control. In embodiments, the standard control is the expression level of CD107a in an NK cell obtained from a subject who does not have leukemia. In embodiments, the standard control is the mean expression level of CD107a in a population of NK cells obtained from a subject who does not have leukemia. The mean expression level may correlate to the mean fluorescent intensity when detected by cytometric methods. The mean expression level may be measured as mean mRNA transcript levels when detected by sequencing methods (e.g. single cell sequencing). In embodiments, the expression level of CD107a is undetectable in the standard control. In embodiments, the presence of CD107a is detected if the expression level of CD107a is measureable by any method known in the art (e.g. cytometric methods, sequencing methods, antibody based methods, etc.).

In embodiments, the presence of CD107a is detected if the expression level of CD107a is elevated relative to CD107a expression in a stage 5 NK cell (e.g. standard control). In embodiments, the presence of CD69 is detected if the expression level of CD107a is elevated relative to CD107a expression in a population of stage 5 NK cells (e.g. standard control) In embodiments, the presence of CD107a is detected if the expression level of CD69 is greater than the mean CD107a expression level in a population of stage 5 NK cells (e.g. standard control).

In embodiments, the population of NK cells has an increased frequency of NK cells that express CD107a relative to a standard control. In embodiments, the standard control is the frequency of NK cells that express CD107a in a population of NK cells obtained from a subject who does not have leukemia. In embodiments, the standard control is the frequency of NK cells expressing CD107a in a population of NK cells obtained from a subject who does not have leukemia, wherein CD107a expression refers to an expression level higher than the mean expression level of the population of cells obtained from the subject who does not have leukemia. In embodiments, the frequency of NK cells that express CD107a in the population of NK cells is elevated by at least 5% relative to the standard control. In embodiments, the frequency of NK cells that express CD107a in the population of NK cells is elevated by at least 10% relative to the standard control. In embodiments, the frequency of NK cells that express CD107a in the population of NK cells is elevated by at least 15% relative to the standard control. In embodiments, the frequency of NK cells that express CD107a in the population of NK cells is elevated by at least 20% relative to the standard control. In embodiments, the frequency of NK cells that express CD107a in the population of NK cells is elevated by at least 25% relative to the standard control. In embodiments, the frequency of NK cells that express CD107a in the population of NK cells is elevated by at least 30% relative to the standard control. In embodiments, the frequency of NK cells that express CD107a in the population of NK cells is elevated by at least 35% relative to the standard control. In embodiments, the frequency of NK cells that express CD107a in the population of NK cells is elevated by at least 40% relative to the standard control. In embodiments, the frequency of NK cells that express CD107a in the population of NK cells is elevated by at least 45% relative to the standard control. In embodiments, the frequency of NK cells that express CD107a in the population of NK cells is elevated by at least 50% relative to the standard control. In embodiments, the frequency of NK cells that express CD107a in the population of NK cells is elevated by at least 55% relative to the standard control. In embodiments, the frequency of NK cells that express CD107a in the population of NK cells is elevated by at least 60% relative to the standard control. In embodiments, the frequency of NK cells that express CD107a in the population of NK cells is elevated by at least 65% relative to the standard control. In embodiments, the frequency of NK cells that express CD107a in the population of NK cells is elevated by at least 75% relative to the standard control. In embodiments, the frequency of NK cells that express CD107a in the population of NK cells is elevated by at least 80% relative to the standard control. In embodiments, the frequency of NK cells that express CD107a in the population of NK cells is elevated by at least 85% relative to the standard control. In embodiments, the frequency of NK cells that express CD107a in the population of NK cells is elevated by at least 90% relative to the standard control. In embodiments, the frequency of NK cells that express CD107a in the population of NK cells is elevated by at least 95% relative to the standard control. In embodiments, the frequency of NK cells that express CD107a in the population of NK cells is elevated by at least 100% relative to the standard control.

In embodiments, the frequency of NK cells that express CD107a in the population of NK cells is elevated by at least 0.1× relative to the standard control. In embodiments, the frequency of NK cells that express CD107a in the population of NK cells is elevated by at least 0.5× relative to the standard control. In embodiments, the frequency of NK cells that express CD107a in the population of NK cells is elevated by at least 1× relative to the standard control. In embodiments, the frequency of NK cells that express CD107a in the population of NK cells is elevated by at least 2× relative to the standard control. In embodiments, the frequency of NK cells that express CD107a in the population of NK cells is elevated by at least 3× relative to the standard control. In embodiments, the frequency of NK cells that express CD107a in the population of NK cells is elevated by at least 4× relative to the standard control. In embodiments, the frequency of NK cells that express CD107a in the population of NK cells is elevated by at least 5× relative to the standard control. In embodiments, the frequency of NK cells that express CD107a in the population of NK cells is elevated by at least 6× relative to the standard control. In embodiments, the frequency of NK cells that express CD107a in the population of NK cells is elevated by at least 7× relative to the standard control. In embodiments, the frequency of NK cells that express CD107a in the population of NK cells is elevated by at least 8× relative to the standard control. In embodiments, the frequency of NK cells that express CD107a in the population of NK cells is elevated by at least 9× relative to the standard control. In embodiments, the frequency of NK cells that express CD107a in the population of NK cells is elevated by at least 10× relative to the standard control. In embodiments, the frequency of NK cells that express CD107a in the population of NK cells is elevated by at least 20× relative to the standard control. In embodiments, the frequency of NK cells that express CD107a in the population of NK cells is elevated by at least 30× relative to the standard control. In embodiments, the frequency of NK cells that express CD107a in the population of NK cells is elevated by at least 40× relative to the standard control. In embodiments, the frequency of NK cells that express CD107a in the population of NK cells is elevated by at least 50× relative to the standard control. In embodiments, the frequency of NK cells that express CD107a in the population of NK cells is elevated by at least 100× relative to the standard control. In embodiments, the frequency of NK cells that express CD107a in the population of NK cells is elevated by at least 150× relative to the standard control. In embodiments, the frequency of NK cells that express CD107a in the population of NK cells is elevated by at least 200× relative to the standard control.

Applicant has discovered that dysfunctional NK cells have impaired cytotoxicity despite their chronic activation. The impaired cytotoxicity may be due to upregulation of checkpoint markers. As used herein, the terms “checkpoint marker” refers to a molecule capable of modulating an immune response, for example the duration or amplitude of an immune response. In embodiments, the checkpoint marker is a negative regulator (e.g. decreases) of an immune response. For example, the checkpoint marker may decrease cytotoxicity of an NK cell, NK cell proliferation, or expression of cytokines and their cognate receptors. In embodiments, a checkpoint marker inhibits maturation of an NK cell. Thus, in embodiments, the method further includes detecting the presence of one or more checkpoint markers in the population of NK cells. In embodiments, the checkpoint marker is Lymphocyte-activation gene 3 (LAG-3), Killer cell lectin-like receptor subfamily G member 1 (KLRG1), Programmed cell death 1 ligand 2 (PD-L2), or combinations thereof. In embodiments, the checkpoint marker is LAG-3. In embodiments, the checkpoint marker is KLRG1. In embodiments, the checkpoint marker is PD-L2.

In embodiments, the presence of a checkpoint marker is detected if the expression level of the checkpoint marker is elevated relative to a standard control. In embodiments, the standard control is the expression level of the checkpoint marker in an NK cell obtained from a subject who does not have leukemia. In embodiments, the standard control is the mean expression level of the checkpoint marker in a population of NK cells obtained from a subject who does not have leukemia. The mean expression level may correlate to mean fluorescent intensity when detected by cytometric methods. The mean expression level may be measured as mean mRNA transcript levels when detected by sequencing methods (e.g. single cell sequencing). In embodiments, the expression level of the checkpoint marker is undetectable in the standard control. In embodiments, the presence of the checkpoint marker is detected if the expression level of the checkpoint marker is measureable by any method known in the art (e.g. cytometric methods, sequencing methods, antibody based methods, etc.).

In embodiments, the presence of a checkpoint marker is detected if the expression level of the checkpoint marker is elevated relative to the checkpoint marker expression level in a stage 5 NK cell (e.g. standard control). In embodiments, the presence of the checkpoint marker is detected if the expression level of the checkpoint marker is elevated relative to the checkpoint marker expression level in a population of stage 5 NK cells (e.g. standard control) In embodiments, the presence of a checkpoint marker is detected if the expression level of the checkpoint marker is greater than the mean checkpoint marker expression level in a population of stage 5 NK cells (e.g. standard control).

In embodiments, the population of NK cells has an increased frequency of NK cells that express the checkpoint marker relative to a standard control. In embodiments, the standard control is the frequency of NK cells that express the checkpoint marker in a population of NK cells obtained from a subject who does not have leukemia. In embodiments, the standard control is the frequency of NK cells expressing the checkpoint marker in a population of NK cells obtained from a subject who does not have leukemia, wherein the checkpoint marker expression refers to an expression level higher than the mean expression level of the population of cells obtained from the subject who does not have leukemia. In embodiments, the frequency of NK cells that express the checkpoint marker in the population of NK cells is elevated by at least 5% relative to the standard control. In embodiments, the frequency of NK cells that express the checkpoint marker in the population of NK cells is elevated by at least 10% relative to the standard control. In embodiments, the frequency of NK cells that express the checkpoint marker in the population of NK cells is elevated by at least 15% relative to the standard control. In embodiments, the frequency of NK cells that express the checkpoint marker in the population of NK cells is elevated by at least 20% relative to the standard control. In embodiments, the frequency of NK cells that express the checkpoint marker in the population of NK cells is elevated by at least 25% relative to the standard control. In embodiments, the frequency of NK cells that express the checkpoint marker in the population of NK cells is elevated by at least 30% relative to the standard control. In embodiments, the frequency of NK cells that express the checkpoint marker in the population of NK cells is elevated by at least 35% relative to the standard control. In embodiments, the frequency of NK cells that express the checkpoint marker in the population of NK cells is elevated by at least 40% relative to the standard control. In embodiments, the frequency of NK cells that express the checkpoint marker in the population of NK cells is elevated by at least 45% relative to the standard control. In embodiments, the frequency of NK cells that express the checkpoint marker in the population of NK cells is elevated by at least 50% relative to the standard control. In embodiments, the frequency of NK cells that express the checkpoint marker in the population of NK cells is elevated by at least 55% relative to the standard control. In embodiments, the frequency of NK cells that express the checkpoint marker in the population of NK cells is elevated by at least 60% relative to the standard control. In embodiments, the frequency of NK cells that express the checkpoint marker in the population of NK cells is elevated by at least 65% relative to the standard control. In embodiments, the frequency of NK cells that express the checkpoint marker in the population of NK cells is elevated by at least 75% relative to the standard control. In embodiments, the frequency of NK cells that express the checkpoint marker in the population of NK cells is elevated by at least 80% relative to the standard control. In embodiments, the frequency of NK cells that express the checkpoint marker in the population of NK cells is elevated by at least 85% relative to the standard control. In embodiments, the frequency of NK cells that express the checkpoint marker in the population of NK cells is elevated by at least 90% relative to the standard control. In embodiments, the frequency of NK cells that express the checkpoint marker in the population of NK cells is elevated by at least 95% relative to the standard control. In embodiments, the frequency of NK cells that express the checkpoint marker in the population of NK cells is elevated by at least 100% relative to the standard control.

Applicant has shown that dysfunctional NK cells overexpress CXCR4. CXCR4 may cause dysfunctional NK cells to migrate, and be a factor in the poor prognosis and increased susceptibility to relapse in leukemia patients having a high level of dysfunctional NK cells. Thus, in embodiments, the method further includes detecting the presence of C-X-C chemokine receptor type 4 (CXCR4) in the population of NK cells.

In embodiments, the presence of CXCR4 is detected if the expression level of CXCR4 is elevated relative to a standard control. In embodiments, the standard control is the expression level of CXCR4 in an NK cell obtained from a subject who does not have leukemia. In embodiments, the standard control is the expression level of CXCR4 of in a population of NK cells obtained from a subject who does not have leukemia. In embodiments, the standard control is the mean expression level of CXCR4 in the population of NK cells obtained from a subject who does not have leukemia. The mean expression level may correlate to mean fluorescent intensity when detected by cytometric methods. The mean expression level may be measured as mean mRNA transcript levels when detected by sequencing methods (e.g. single cell sequencing). In embodiments, the expression level of CXCR4 is undetectable in the standard control. In embodiments, the presence of CXCR4 is detected if the expression level of CXCR4 is measureable by any method known in the art (e.g. cytometric methods, sequencing methods, antibody based methods, etc.).

In embodiments, the presence of CXCR4 is detected if the expression level of the CXCR4 is elevated relative to a standard control. In embodiments, the standard control is the expression level of the CXCR4 in an NK cell obtained from a subject who does not have leukemia. In embodiments, the standard control is the mean expression level of the CXCR4 in a population of NK cells obtained from a subject who does not have leukemia. The mean expression level may be correlated to mean fluorescent intensity when detected by cytometric methods. The mean expression level may be measured as mean CXCR4 mRNA transcript levels when detected by sequencing methods (e.g. single cell sequencing). In embodiments, the expression level of the CXCR4 is undetectable in the standard control. In embodiments, the presence of the CXCR4 is detected if the expression level of the CXCR4 is measureable by any method known in the art (e.g. cytometric methods, sequencing methods, antibody based methods, etc.).

In embodiments, the presence of CXCR4 is detected if the expression level of CXCR4 is elevated relative to CXCR4 expression in a stage 5 NK cell (e.g. standard control). In embodiments, the presence of CXCR4 is detected if the expression level of CXCR4 is elevated relative to CXCR4 expression in a population of stage 5 NK cells (e.g. standard control) In embodiments, the presence of CXCR4 is detected if the expression level of CXCR4 is greater than the mean CXCR4 expression level in a population of stage 5 NK cells (e.g. standard control).

In embodiments, the population of NK cells has an increased frequency of NK cells that express the CXCR4 relative to a standard control. In embodiments, the standard control is the frequency of NK cells that express the CXCR4 in a population of NK cells obtained from a subject who does not have leukemia. In embodiments, the standard control is the frequency of NK cells expressing the CXCR4 in a population of NK cells obtained from a subject who does not have leukemia, wherein the CXCR4 expression refers to an expression level higher than the mean expression level of the population of cells obtained from the subject who does not have leukemia. In embodiments, the frequency of NK cells that express the CXCR4 in the population of NK cells is elevated by at least 5% relative to the standard control. In embodiments, the frequency of NK cells that express the CXCR4 in the population of NK cells is elevated by at least 10% relative to the standard control. In embodiments, the frequency of NK cells that express the CXCR4 in the population of NK cells is elevated by at least 15% relative to the standard control. In embodiments, the frequency of NK cells that express the CXCR4 in the population of NK cells is elevated by at least 20% relative to the standard control. In embodiments, the frequency of NK cells that express the CXCR4 in the population of NK cells is elevated by at least 25% relative to the standard control. In embodiments, the frequency of NK cells that express the CXCR4 in the population of NK cells is elevated by at least 30% relative to the standard control. In embodiments, the frequency of NK cells that express the CXCR4 in the population of NK cells is elevated by at least 35% relative to the standard control. In embodiments, the frequency of NK cells that express the CXCR4 in the population of NK cells is elevated by at least 40% relative to the standard control. In embodiments, the frequency of NK cells that express the CXCR4 in the population of NK cells is elevated by at least 45% relative to the standard control. In embodiments, the frequency of NK cells that express the CXCR4 in the population of NK cells is elevated by at least 50% relative to the standard control. In embodiments, the frequency of NK cells that express the CXCR4 in the population of NK cells is elevated by at least 55% relative to the standard control. In embodiments, the frequency of NK cells that express the CXCR4 in the population of NK cells is elevated by at least 60% relative to the standard control. In embodiments, the frequency of NK cells that express the CXCR4 in the population of NK cells is elevated by at least 65% relative to the standard control. In embodiments, the frequency of NK cells that express the CXCR4 in the population of NK cells is elevated by at least 75% relative to the standard control. In embodiments, the frequency of NK cells that express the CXCR4 in the population of NK cells is elevated by at least 80% relative to the standard control. In embodiments, the frequency of NK cells that express the CXCR4 in the population of NK cells is elevated by at least 85% relative to the standard control. In embodiments, the frequency of NK cells that express the CXCR4 in the population of NK cells is elevated by at least 90% relative to the standard control. In embodiments, the frequency of NK cells that express the CXCR4 in the population of NK cells is elevated by at least 95% relative to the standard control. In embodiments, the frequency of NK cells that express the CXCR4 in the population of NK cells is elevated by at least 100% relative to the standard control.

In embodiments, the frequency of NK cells that express CXCR4 in the population of NK cells is elevated by at least 0.1× relative to the standard control. In embodiments, the frequency of NK cells that express CXCR4 in the population of NK cells is elevated by at least 0.5× relative to the standard control. In embodiments, the frequency of NK cells that express CXCR4 in the population of NK cells is elevated by at least 1× relative to the standard control. In embodiments, the frequency of NK cells that express CXCR4 in the population of NK cells is elevated by at least 2× relative to the standard control. In embodiments, the frequency of NK cells that express CXCR4 in the population of NK cells is elevated by at least 3× relative to the standard control. In embodiments, the frequency of NK cells that express CXCR4 in the population of NK cells is elevated by at least 4× relative to the standard control. In embodiments, the frequency of NK cells that express CXCR4 in the population of NK cells is elevated by at least 5× relative to the standard control. In embodiments, the frequency of NK cells that express CXCR4 in the population of NK cells is elevated by at least 6× relative to the standard control. In embodiments, the frequency of NK cells that express CXCR4 in the population of NK cells is elevated by at least 7× relative to the standard control. In embodiments, the frequency of NK cells that express CXCR4 in the population of NK cells is elevated by at least 8× relative to the standard control. In embodiments, the frequency of NK cells that express CXCR4 in the population of NK cells is elevated by at least 9× relative to the standard control. In embodiments, the frequency of NK cells that express CXCR4 in the population of NK cells is elevated by at least 10× relative to the standard control. In embodiments, the frequency of NK cells that express CXCR4 in the population of NK cells is elevated by at least 20× relative to the standard control. In embodiments, the frequency of NK cells that express CXCR4 in the population of NK cells is elevated by at least 30× relative to the standard control. In embodiments, the frequency of NK cells that express CXCR4 in the population of NK cells is elevated by at least 40× relative to the standard control. In embodiments, the frequency of NK cells that express CXCR4 in the population of NK cells is elevated by at least 50× relative to the standard control. In embodiments, the frequency of NK cells that express CXCR4 in the population of NK cells is elevated by at least 100× relative to the standard control. In embodiments, the frequency of NK cells that express CXCR4 in the population of NK cells is elevated by at least 150× relative to the standard control. In embodiments, the frequency of NK cells that express CXCR4 in the population of NK cells is elevated by at least 200× relative to the standard control.

For the methods provided herein, in embodiments, the population of NK cells are enriched from a population of bone marrow mononuclear cells (BMMC) obtained from the subject or a population of peripheral blood mononuclear cells (PBMC) obtained from the subject. “Enriched” as used herein refers to separating non-NK cells (e.g. T cells, B cells, etc.) from a population of cells including NK cells and non-NK cells. In embodiments, non-NK cells may be removed by antibody based methods, for example by binding an antibody to a protein expressed on the surface of the non-NK cell and selecting the antibody-bound cell for removal. Similarly, NK cells may be selected by contacting a population of cells including NK cells and non-NK cells with a mixture of antibodies that bind proteins expressed on the surface of NK cells and selecting the antibodies that bind NK cells. Methods for enriching cells include cytometric methods, chromatographic methods, gravity based purification, and magnetic separation. In embodiments, the population of NK cells are enriched from BMMC obtained from the subject. In embodiments, the population of NK cells are enriched from PBMC obtained from the subject.

In embodiments, a population of cells obtained from a subject (e.g. a subject having leukemia, a subject who does not have leukemia) includes about 5% to about 100% NK cells (e.g. the population of NK cells) following an enrichment step. In embodiments, the population of cells obtained from a subject includes about 10% to about 100% NK cells. In embodiments, the population of cells obtained from a subject includes about 15% to about 100% NK cells. In embodiments, the population of cells obtained from a subject includes about 20% to about 100% NK cells. In embodiments, the population of cells obtained from a subject includes about 25% to about 100% NK cells. In embodiments, the population of cells obtained from a subject includes about 30% to about 100% NK cells. In embodiments, the population of cells obtained from a subject includes about 35% to about 100% NK cells. In embodiments, the population of cells obtained from a subject includes about 40% to about 100% NK cells. In embodiments, the population of cells obtained from a subject includes about 45% to about 100% NK cells. In embodiments, the population of cells obtained from a subject includes about 50% to about 100% NK cells. In embodiments, the population of cells obtained from a subject includes about 55% to about 100% NK cells. In embodiments, the population of cells obtained from a subject includes about 60% to about 100% NK cells. In embodiments, the population of cells obtained from a subject includes about 65% to about 100% NK cells. In embodiments, the population of cells obtained from a subject includes about 70% to about 100% NK cells. In embodiments, the population of cells obtained from a subject includes about 75% to about 100% NK cells. In embodiments, the population of cells obtained from a subject includes about 80% to about 100% NK cells. In embodiments, the population of cells obtained from a subject includes about 85% to about 100% NK cells. In embodiments, the population of cells obtained from a subject includes about 90% to about 100% NK cells. In embodiments, the population of cells obtained from a subject includes about 95% to about 100% NK cells.

In embodiments, the population of cells obtained from a subject includes about 5% to about 95% NK cells following an enrichment step. In embodiments, the population of cells obtained from a subject includes about 5% to about 90% NK cells. In embodiments, the population of cells obtained from a subject includes about 5% to about 85% NK cells. In embodiments, the population of cells obtained from a subject includes about 5% to about 80% NK cells. In embodiments, the population of cells obtained from a subject includes about 5% to about 75% NK cells. In embodiments, the population of cells obtained from a subject includes about 5% to about 70% NK cells. In embodiments, the population of cells obtained from a subject includes about 5% to about 65% NK cells. In embodiments, the population of cells obtained from a subject includes about 5% to about 60% NK cells. In embodiments, the population of cells obtained from a subject includes about 5% to about 55% NK cells. In embodiments, the population of cells obtained from a subject includes about 5% to about 50% NK cells. In embodiments, the population of cells obtained from a subject includes about 5% to about 45% NK cells. In embodiments, the population of cells obtained from a subject includes about 5% to about 40% NK cells. In embodiments, the population of cells obtained from a subject includes about 5% to about 35% NK cells. In embodiments, the population of cells obtained from a subject includes about 5% to about 20% NK cells. In embodiments, the population of cells obtained from a subject includes about 5% to about 15% NK cells. In embodiments, the population of cells obtained from a subject includes about 5% to about 10% NK cells. In embodiments, the population of cells obtained from a subject includes about 5%, 10%, 15%, 20%, 25%, 30%, 35%, 40%, 45%, 50%, 55%, 60%, 65%, 70%, 75%, 80% 85%, 90%, 95%, or 100% NK cells following an enrichment step.

In embodiments, the leukemia is acute lymphoblastic leukemia (ALL). In embodiments, the ALL is T-cell ALL (T-ALL) or B-cell ALL (B-ALL). In embodiments, the ALL is T-ALL. In embodiments, the ALL is B-ALL.

For the methods provided herein, in embodiments, the detecting includes a cytometric method or measuring RNA transcript levels. In embodiments, the detecting includes a cytometric method. “Cytometric method” is used in accordance to its plain ordinary meaning in the art and refers to a method of identifying cells (e.g. NK cells) by detecting physical characteristics of the cell. For example, a cell may be identified by its size, morphology, protein expression, or level of protein expression, etc. For example, a cytometric method may include contacting a population of NK cells with an antibody or a plurality of antibodies that bind a protein expressed by a subset of the NK cells (e.g. dysfunctional NK cells). Thus, the subset of cells (e.g. dysfunctional NK cells) may be identified by detecting antibody binding to a protein (e.g. cytokine) expressed by the subset of cells (e.g. dysfunctional NK cells). In embodiments, the antibody includes a detectable label (e.g. a fluorescent label). In embodiments, the detectable label is a fluorescent label. The expression level of the protein may be correlated to the intensity of the signal from the detectable label. For example, a higher fluorescent signal intensity correlates to a higher expression level of the protein. The subset of cells expressing the protein may further be analyzed by using a second antibody that binds specifically to a second marker expressed by the cell. Subsets of cells (e.g. dysfunctional NK cells) within the population of NK cells may be identified by gating. “Gating” refers to selection of a specific cell population. For example, CD56^{bright} NK cells (e.g. dysfunctional NK cells) may be selected by identifying a subset of cells that have a high fluorescent intensity (e.g. high expression level) when bound to an anti-CD56 antibody conjugated to a fluorescent label.

In embodiments, the cytometric method is fluorescent activated cell sorting (FACS). As described above, the population of NK cells may be contacted with an antibody or a plurality of antibodies that bind a protein expressed by a subset of the NK cells (e.g. dysfunctional NK cells). The antibody typically includes a detectable label, for example, a fluorescent label. In embodiments, FACS includes separating cells in the population of NK cells by enclosing the cells in liquid droplets, wherein each droplet includes a single cell. In embodiments, the liquid droplets are labeled with electric charges and sorted by an external electric field. In embodiments, the cells are sorted based on the intensity of the fluorescent signal, wherein intensity of the fluorescent signal correlates to presence of protein expression or level of protein expression (e.g. elevated protein expression). In embodiments, the subset of cells (e.g. dysfunctional NK cells) expressing the protein (e.g. expression events) can be separated from cells not expressing the protein (e.g. NK cells that are not dysfunctional NK cells). The separated cells can include only one cell or more than one cell (e.g., 10, 100, 1000, 10,000 or 100,000 cells). In embodiments, gene expression may be measured in the separated cells expressing the protein by, e.g., RT-PCR or a transcriptomic analysis of RNA in the cells.

In embodiments, the cytometric method is minimal residual disease (MRD) by multiple parameter flow cytometry. MRD is used in accordance to its ordinary meaning in the art and refers to a method of identifying subsets of cells within a population of cells obtained from a patient with cancer, wherein the subset of cells produce molecules indicative of pathogenity. For example, MRD may be used to detect cancer cells in a population of BMMC or PBMC cells obtained from a patient with leukemia. MRD may be used to monitor the effectiveness of a therapeutic; for example, a decrease in the frequency of cancer cells in a population of cancer cells after administration of a therapeutic may indicate that the therapeutic is effective for treating cancer. In another example, MRD may be used to diagnose cancer. For example, MRD may be used to detect the frequency of dysfunctional NK cells in a population of NK cells obtained from a subject, wherein an increased frequency of dysfunctional NK cells relative to a standard control indicates that the subject has cancer. In embodiments, MRD includes contacting a population of NK cells with a plurality of antibodies specific for proteins expressed by dysfunctional NK cells (e.g. CD56, CD69, cytokines, etc.). MRD is contemplated to be effective for detecting residual numbers of cells (e.g. dysfunctional NK cells) in a population of cells. For example, MRD may be useful for detecting dysfunctional NK cells when less than 25%, 20%, 15%, 10%, 5%, 2.5%, 1% or 0.5% of the population of cells are dysfunctional NK cells. For example, MRD may be useful for accurately detecting dysfunctional NK cells in a population of NK cells by contacting the population of NK cells with a plurality of antibodies specific for multiple proteins expressed by the dysfunctional NK cells.

For the methods provided herein, in embodiments, the detecting includes a sequencing method. In embodiments, the sequencing method includes RNA sequencing (RNA-seq), DNA sequencing, epigenetic sequencing, or protein sequencing. In embodiments, the sequencing method includes RNA-seq. In embodiments, the sequencing method includes DNA sequencing. In embodiments, the sequencing method includes epigenetic sequencing. In embodiments, the sequencing method includes protein sequencing. In embodiments, RNA-seq includes measuring mRNA transcript levels of one or more genes expressed by dysfunctional NK cells (e.g. CD56, CD69, a cytokine, etc.). In embodiments, measuring RNA transcript levels includes single cell sequencing methods. In embodiments, measuring RNA transcript levels includes mnicroarray analysis or reverse transcription polymerase chain reaction (RT-PCR).

In embodiments, 50% or more of the population of NK cells obtained from the subject are dysfunctional NK cells. For example, 50% or more of the NK cells in the population of NK cells have a) an elevated expression level of CD56 relative to a standard control; b) an elevated expression level of CD94 relative to the standard control; c) an elevated ratio of CD11b⁻CD27− NK cells to CD11b⁺CD27− NK cells relative to the standard control; d) express CD69; e) express a cytokine; or f) have calcium (Ca²⁺) mobilization. For example, 50% or more of the NK cells in the population of NK cells have a) an elevated expression level of CD56 relative to a standard control; b) an elevated expression level of CD94 relative to the standard control; c) an elevated ratio of CD11b⁻CD27− NK cells to CD11b⁺CD27− NK cells relative to the standard control; d) express CD69; e) express a cytokine; and f) have calcium (Ca²⁺) mobilization. In embodiments, 50% or more of the NK cells in the population of NK cells have an elevated expression level of CD56 relative to a standard control. In embodiments, 50% or more of the NK cells in the population of NK cells have an elevated expression level of CD94 relative to the standard control. In embodiments, 50% or more of the NK cells in the population of NK cells have an elevated ratio of CD11b-CD27− NK cells to CD11b⁺CD27− NK cells relative to the standard control. In embodiments, 50% or more of the NK cells in the population of NK cells express CD69. In embodiments, 50% or more of the NK cells in the population of NK cells express a cytokine. In embodiments, the cytokine is IFN-γ, TNF, GM-CSF, MIP-10, IL-2, or combinations thereof. In embodiments, 50% or more of the NK cells in the population of NK cells have calcium (Ca²¹) mobilization.

In embodiments, the method further includes administering to the subject an effective amount of allogeneic NK cells. In embodiments, the allogeneic NK cells are obtained from a subject who does not have leukemia. In embodiments, the allogeneic NK cells are not dysfunctional NK cells.

In embodiments, the methods further include performing a diagnostic test. Diagnostic tests for leukemia are well known in the art and include complete blood count (CBC), microscopic evaluation of the blood, or by flow cytometry. In embodiments, the method of detecting leukemia includes, but is not limited to a bone marrow biopsy, imaging tests (e.g., X-ray, computed tomography (CT) scan, CT-guided needle biopsy, magnetic resonance imaging (MRI) scan, and positron emission tomography (PET) scan).

### Methods of Treatment

Allogeneic NK cells are contemplated to be an effective therapeutic for treating subjects with leukemia. Particularly, the decreased frequency of NK cells and increased proportion of dysfunctional NK cells in B-ALL and T-ALL subjects presents challenges in obtaining a sufficient population of therapeutically effective autologous NK cells for treating B/T-ALL subjects. As described throughout the specification, including in the examples and figures, dysfunctional NK cells from ALL subjects have decreased capability in cell killing compared to NK cells from healthy donors (subjects who do not have leukemia). Thus, provided herein are methods for treating leukemia including administering allogeneic NK cells to the subject when over 20%, 30%, 40%, 50%, 60%, 70%, 80%, or 80% of the NK cells in a population of NK cells from the subject are dysfunctional NK cells. As described by the current invention, a dysfunctional NK cell from a leukemia patient has an elevated expression level of CD56 relative to a standard control; an elevated expression level of CD94 relative to the standard control; expresses CD69; expresses one or more cytokines as described herein, or has calcium mobilization. In embodiments, a higher ratio of CD11b-CD27− NK cells to CD11b⁺CD27− NK cells in a population of NK cells obtained from the subject is indicative of dysfunctional NK cells. In an aspect is provided a method of treating leukemia in a subject in need thereof, including administering to the subject an effective amount of allogeneic NK cells, wherein 50% or more of a population of NK cells obtained from the subject are dysfunctional NK cells. In embodiments, the method includes obtaining the population of NK cells from the subject and identifying the dysfunctional NK cells prior to administering the effective amount of allogeneic NK cells. In embodiments, the identifying the dysfunctional NK cells includes detecting in the population of NK cells: (a) an elevated expression level of CD56 relative to a standard control; (b) an elevated expression level of CD94 relative to the standard control; (c) an elevated ratio of CD11b⁻CD27− cells to CD11b⁺CD27− cells relative to the standard control; (d) the presence of CD69; (e) the presence of a cytokine; or (f) calcium (Ca2+) mobilization. In embodiments, the identifying the dysfunctional NK cells includes detecting in the population of NK cells: (a) an elevated expression level of CD56 relative to a standard control; (b) an elevated expression level of CD94 relative to the standard control; (c) an elevated ratio of CD11b⁻CD27− cells to CD11b⁺CD27− cells relative to the standard control; (d) the presence of CD69; (e) the presence of a cytokine; and (f) calcium (Ca2+) mobilization.

In embodiments, the cytokine is Interferon gamma (IFN-γ), Tumor necrosis factor (TNF), Granulocyte-macrophage colony-stimulating factor (GM-CSF), Macrophage inflammatory protein 1 beta (MIP-10), Interleukin-2 (IL-2), or combinations thereof. In embodiments, the cytokine is IFN-γ, TNF, GM-CSF, or combinations thereof.

In embodiments, the method further includes detecting the absence of DNAX Accessory Molecule-1 (DNAM 1), Killer cell immunoglobulin-like receptor 2DL1 (KIR2DL1), CD57, Siglec-7, or combinations thereof in the population of NK cells. In embodiments, the method further includes further includes detecting the presence of cytotoxic granules in said population of NK cells. In embodiments, detecting the presence of cytotoxic granules includes detecting the presence of perforin (PRF) or granzyme B (GZMB) in said population of NK cells.

In embodiments, the method includes detecting the presence of one or more activation markers in said population of NK cells. In embodiments, the activation marker is CD107a. In embodiments, the method includes detecting the presence of one or more checkpoint markers in the population of NK cells. In embodiments, the checkpoint marker is Lymphocyte-activation gene 3 (LAG-3), Killer cell lectin-like receptor subfamily G member 1 (KLRG1), Programmed cell death 1 ligand 2 (PD-L2), or combinations thereof. In embodiments, the method further includes detecting the presence of C-X-C chemokine receptor type 4 (CXCR4) in the population of NK cells.

In embodiments, the population of NK cells are enriched from bone marrow mononuclear cells (BMMC) obtained from the subject or peripheral blood mononuclear cells (PBMC) obtained from the subject. In embodiments, the leukemia is acute lymphoblastic leukemia (ALL). In embodiments, the detecting includes a cytometric method or measuring RNA transcript levels.

In embodiments, the subject previously received treatment for leukemia. In embodiments, the subject was non-responsive to the previous treatment. The term “non-responsive,” as used herein, refers to the instance when treatment of a subject does not result in beneficial effect. The beneficial effect may be a reduction of symptoms, slowing the rate of cancer cell growth, cancer cell killing, or a combination thereof. In embodiments, the subject was previously in remission and has relapsed. In embodiments, the subject has relapsed. The terms “recurrence” or “relapse,” as used herein, refers to when a cancer returns after a period of remission. For example, relapse may refer to detection of cancer cells may after a period when cancer cells were previously undetectable in the subject. Relapse may refer to the return of cancer symptoms after a period of being symptom-free. Relapse generally occurs due to residual numbers of cancer cells that fail to be eliminated or eradicated by a cancer therapeutic (e.g. chemotherapy). After treatment, the residual cancer cells may multiply and grow in amounts large enough to cause symptoms or be detected by diagnostic tests. In embodiments, relapse may recur in the same part of the body, generally referred to as local recurrence. In an embodiment, relapse may recur near where the primary cancer was located, generally referred to as regional recurrence. In an embodiment, relapse may recur in another part of the body, generally referred to as distant recurrence.

Populations of allogeneic NK cells used for the method provided herein include a lower proportion of dysfunctional NK cells relative to the subject who has leukemia. In embodiments, there are fewer than about 40%, 30%, 20%, 18%, 16%, 14%, 12%, 10%, 8%, 6%, 4%, 2%, or 1% dysfunctional NK cells in a population of allogeneic cells. In embodiments, there are fewer than about 10% dysfunctional NK cells in a population of allogeneic cells. In embodiments, there are fewer than about 8% dysfunctional NK cells in a population of allogeneic cells. In embodiments, there are fewer than about 6% dysfunctional NK cells in a population of allogeneic cells. In embodiments, there are fewer than about 5% dysfunctional NK cells in a population of allogeneic cells. In embodiments, there are fewer than about 4% dysfunctional NK cells in a population of allogeneic cells. In embodiments, there are fewer than about 3% dysfunctional NK cells in a population of allogeneic cells. In embodiments, there are fewer than about 2% dysfunctional NK cells in a population of allogeneic cells. In embodiments, there are fewer than about 1% dysfunctional NK cells in a population of allogeneic cells. In embodiments, there are fewer than about 0.5% dysfunctional NK cells in a population of allogeneic cells. In embodiments, there are fewer than about 0.1% dysfunctional NK cells in a population of allogeneic cells. In embodiments, there are fewer than about 0.5% dysfunctional NK cells in a population of allogeneic cells. In embodiments, there are fewer than about 0.01% dysfunctional NK cells in a population of allogeneic cells. In embodiments, dysfunctional NK cells are undetectable in the population of allogeneic cells. In embodiments, the allogeneic NK cells are obtained from a subject who does not have leukemia. In embodiments, the allogeneic NK cells do not include dysfunctional NK cells. In embodiments, the allogeneic NK cells are not dysfunctional NK cells.

Allogeneic NK cell therapeutics are contemplated to be particularly effective for patients who have a high proportion of dysfunctional NK cells (e.g. cells with decreased cancer cell killing capability). Thus, in an aspect, a method of treating leukemia in a subject in need thereof is provided, the method including: a) identifying dysfunctional natural killer (NK) cells in a population of NK cells obtained from the subject; and b) administering to the subject an effective amount of allogeneic NK cells; wherein 50% or more of the population of NK cells are dysfunctional NK cells. In embodiments, identifying the dysfunctional NK cells includes detecting in the population of NK cells: (a) an elevated expression level of CD56 relative to a standard control; (b) an elevated expression level of CD94 relative to the standard control; (c) an elevated ratio of CD11b⁻CD27− cells to CD11b⁺CD27− cells relative to the standard control; (d) the presence of CD69; (e) the presence of a cytokine; or (f) calcium (Ca2+) mobilization; thereby identifying the dysfunctional NK cells in the subject. In embodiments, identifying the dysfunctional NK cells includes detecting in the population of NK cells: (a) an elevated expression level of CD56 relative to a standard control; (b) an elevated expression level of CD94 relative to the standard control; (c) an elevated ratio of CD11b⁻CD27− cells to CD11b⁺CD27− cells relative to the standard control; (d) the presence of CD69; (e) the presence of a cytokine; and (f) calcium (Ca2+) mobilization; thereby identifying the dysfunctional NK cells in the subject.

In embodiments, the cytokine is Interferon gamma (IFN-γ), Tumor necrosis factor (TNF), Granulocyte-macrophage colony-stimulating factor (GM-CSF), Macrophage inflammatory protein 1 beta (MIP-10), Interleukin-2 (IL-2), or combinations thereof. In embodiments, the cytokine is IFN-γ, TNF, GM-CSF, or combinations thereof.

In embodiments, the method further includes detecting the absence of DNAX Accessory Molecule-1 (DNAM 1), Killer cell immunoglobulin-like receptor 2DL1 (KIR2DL1), CD57, Siglec-7, or combinations thereof in the population of NK cells. In embodiments, the method further includes further includes detecting the presence of cytotoxic granules in said population of NK cells. In embodiments, detecting the presence of cytotoxic granules includes detecting the presence of perforin (PRF) or granzyme B (GZMB) in said population of NK cells.

In embodiments, the method includes detecting the presence of one or more activation markers in said population of NK cells. In embodiments, the activation marker is CD107a. In embodiments, the method includes detecting the presence of one or more checkpoint markers in the population of NK cells. In embodiments, the checkpoint marker is Lymphocyte-activation gene 3 (LAG-3), Killer cell lectin-like receptor subfamily G member 1 (KLRG1), Programmed cell death 1 ligand 2 (PD-L2), or combinations thereof. In embodiments, the method further includes detecting the presence of C-X-C chemokine receptor type 4 (CXCR4) in the population of NK cells.

In embodiments, the population of NK cells are enriched from bone marrow mononuclear cells (BMMC) obtained from the subject or peripheral blood mononuclear cells (PBMC) obtained from the subject. In embodiments, the leukemia is acute lymphoblastic leukemia (ALL). In embodiments, the detecting includes a cytometric method or measuring RNA transcript levels.

In embodiments, the subject was non-responsive to the previous treatment. In embodiments, the subject has relapsed.

In embodiments, the allogeneic NK cells are obtained from a subject who does not have leukemia. In embodiments, the allogeneic NK cells are not dysfunctional NK cells.

Applicant has found that NK cells engineered to overexpress proteins (e.g. interferons, IL-15, etc.) that increase cytotoxic effects of said NK cells are effective for treating leukemia in subjects with dysfunctional NK cells. For example, Applicant has demonstrated that NK cells engineered to overexpress Type I IFN- and IL-15 are effective for killing ALL cancer cells. Applicant has further shown that the engineered NK cells have increased cancer cell killing ability compared to non-engineered NK cells. As used herein, “engineered NK cells” refer to NK cells including a nucleic acid sequence encoding a Cas9 protein (e.g. SpCas9, SaCas9, StCas9, NmCas9, FnCas9, CjCas9, ScCas9, SauriCas9, eSpCas9, HypaCas9, xCas9, or dCas9), wherein the nucleic acid sequence is integrated into the NK cell genome. For example, an NK cell may be transduced (e.g. via a lentiviral vector) with a first nucleic acid encoding a Cas9 protein and a second nucleic acid encoding a single guide RNA (sgRNA) targeting a gene of interest (e.g. target gene). In embodiments, the first nucleic acid and/or second nucleic acid sequence is integrated into the NK cell genome, thereby producing the engineered NK cell. The first nucleic acid may encode a Cas9 protein (e.g. dCas) fused to a transcriptional activator (e.g. dCAS9-VP64) or transcriptional inactivator (dCAS9-KRAB). Administration of the engineered NK cell to a subject having leukemia therefore may modify expression levels of genes associated with disease pathways, for example, genes that are repressed by the MYC oncogene. For example, an engineered NK cell including a nucleic acid encoding dCas9-VP64 and sgRNA targeting Type I IFN and/or IL-15 can overexpress Type I IFN- and IL-15, thereby increasing cytotoxic effects of the engineered NK cell. Thus, in an aspect is provided a method of treating a subject having leukemia, including administering an effective amount of an engineered NK cell to the subject, wherein 10% of more of a population of NK cells obtained from the subject are dysfunctional NK cells. Engineered NK cells are described in greater detail in PCT/US2021/056714, which is incorporated herein in its entirety and for all purposes.

In embodiments, the method incudes obtaining the population of NK cells from the subject. In embodiments, the method includes identifying dysfunctional NK cells in the population of NK cells obtained from the subject, wherein the identifying includes detecting in the population of NK cells (a) an elevated expression level of CD56 relative to a standard control; (b) an elevated expression level of CD94 relative to the standard control; (c) an elevated ratio of CD11b⁻CD27− cells to CD11b⁺CD27− cells relative to the standard control; (d) the presence of CD69; (e) the presence of a cytokine; or (f) calcium (Ca²¹) mobilization. In embodiments, the dysfunctional NK cells are identified prior to administration of the engineered NK cells.

In embodiments, the population of NK cells includes at least about 10% dysfunctional NK cells. In embodiments, the population of NK cells includes at least about 15% dysfunctional NK cells. In embodiments, the population of NK cells includes at least about 20% dysfunctional NK cells. In embodiments, the population of NK cells includes at least about 25% dysfunctional NK cells. In embodiments, the population of NK cells includes at least about 30% dysfunctional NK cells. In embodiments, the population of NK cells includes at least about 35% dysfunctional NK cells. In embodiments, the population of NK cells includes at least about 40% dysfunctional NK cells. In embodiments, the population of NK cells includes at least about 45% dysfunctional NK cells. In embodiments, the population of NK cells includes at least about 50% dysfunctional NK cells. In embodiments, the population of NK cells includes at least about 55% dysfunctional NK cells. In embodiments, the population of NK cells includes at least about 60% dysfunctional NK cells. In embodiments, the population of NK cells includes at least about 65% dysfunctional NK cells. In embodiments, the population of NK cells includes at least about 70% dysfunctional NK cells. In embodiments, the population of NK cells includes at least about 75% dysfunctional NK cells. In embodiments, the population of NK cells includes at least about 80% dysfunctional NK cells. In embodiments, the population of NK cells includes at least about 85% dysfunctional NK cells. In embodiments, the population of NK cells includes at least about 90% dysfunctional NK cells. In embodiments, the population of NK cells includes at least about 95% dysfunctional NK cells. In embodiments, the population of NK cells includes at least about 98% dysfunctional NK cells. In embodiments, the population of NK cells includes at least about 99% dysfunctional NK cells.

In embodiments, the target gene is IL-15, IFNα2, IFNα1, IFNβ1, STAT1, STAT2, IFNAR 1, IFNAR 2, NKG2D, NKp46, DNAM-1 or CD96. In embodiments, the target gene is IL-15. In embodiments, the target gene is IFNα2. In embodiments, the target gene is IFNα1. In embodiments, the target gene is IFNβ1. In embodiments, the target gene is STAT1. In embodiments, the target gene is STAT2. In embodiments, the target gene is IFNAR 1. In embodiments, the target gene is IFNAR 2. In embodiments, the target gene is NKG2D. In embodiments, the target gene is NKp46. In embodiments, the target gene is DNAM-1. In embodiments, the target gene is CD96.

In embodiments, the engineered NK cells are allogeneic NK cells. In embodiments, the engineered NK cells are autologous NK cells. In embodiments, the engineered NK cells are obtained from a subject who does not have cancer. In embodiments, the engineered NK cells are obtained from a subject who does not leukemia. In embodiments, the engineered NK cells do not include dysfunctional NK cells. In embodiments, the engineered NK cells are not dysfunctional NK cells.

### Methods of Determining Survival or Relapse

Applicant has demonstrated that high levels of dysfunctional NK cells in a subject having leukemia is indicative of decreased probability of survival or increased probability of relapse. Without wishing to be bound by theory, decreased survival and increased relapse rates may be due to decreased capability of the dysfunctional NK cells to kill cancer cells, thereby allowing cancer cells to escape NK cell surveillance. In an aspect is provided a method of determining a probability of survival or relapse in a subject having leukemia, including identifying dysfunctional natural killer (NK) cells in a population of NK cells obtained from the subject, wherein 50% or more of the population of NK cells are dysfunctional NK cells indicates that the subject has decreased probability of survival or increased probability of relapse relative to a subject wherein less than 50% of the population of NK cells are dysfunctional NK cells. In embodiments, the subject having less than 50% dysfunction NK cells has leukemia. In embodiments, the subject having less than 50% dysfunction NK cells has has B-ALL. In embodiments, the subject having less than 50% dysfunction NK cells has has T-ALL. In embodiments, the subject having less than 50% dysfunction NK cells is in remission from the leukemia.

In embodiments, a subject having 50% or more dysfunctional NK cells in a population of NK cells has at least about 5% decreased probability of survival relative to a subject who has less than 50% dysfunctional NK cells in a population of NK cells. In embodiments, a subject having 50% or more dysfunctional NK cells in a population of NK cells has at least about 10% decreased probability of survival relative to a subject who has less than 50% dysfunctional NK cells in a population of NK cells. In embodiments, a subject having 50% or more dysfunctional NK cells in a population of NK cells has at least about 15% decreased probability of survival relative to a subject who has less than 50% dysfunctional NK cells in a population of NK cells. In embodiments, a subject having 50% or more dysfunctional NK cells in a population of NK cells has at least about 20% decreased probability of survival relative to a subject who has less than 50% dysfunctional NK cells in a population of NK cells. In embodiments, a subject having 50% or more dysfunctional NK cells in a population of NK cells has at least about 25% decreased probability of survival relative to a subject who has less than 50% dysfunctional NK cells in a population of NK cells. In embodiments, a subject having 50% or more dysfunctional NK cells in a population of NK cells has at least about 30% decreased probability of survival relative to a subject who has less than 50% dysfunctional NK cells in a population of NK cells. In embodiments, a subject having 50% or more dysfunctional NK cells in a population of NK cells has at least about 30% decreased probability of survival relative to a subject who has less than 50% dysfunctional NK cells in a population of NK cells. In embodiments, a subject having 50% or more dysfunctional NK cells in a population of NK cells has at least about 30% decreased probability of survival relative to a subject who has less than 50% dysfunctional NK cells in a population of NK cells. In embodiments, a subject having 50% or more dysfunctional NK cells in a population of NK cells has at least about 30% decreased probability of survival relative to a subject who has less than 50% dysfunctional NK cells in a population of NK cells. In embodiments, a subject having 50% or more dysfunctional NK cells in a population of NK cells has at least about 35% decreased probability of survival relative to a subject who has less than 50% dysfunctional NK cells in a population of NK cells. In embodiments, a subject having 50% or more dysfunctional NK cells in a population of NK cells has at least about 40% decreased probability of survival relative to a subject who has less than 50% dysfunctional NK cells in a population of NK cells. In embodiments, a subject having 50% or more dysfunctional NK cells in a population of NK cells has at least about 45% decreased probability of survival relative to a subject who has less than 50% dysfunctional NK cells in a population of NK cells. In embodiments, a subject having 50% or more dysfunctional NK cells in a population of NK cells has at least about 50% decreased probability of survival relative to a subject who has less than 50% dysfunctional NK cells in a population of NK cells. In embodiments, a subject having 50% or more dysfunctional NK cells in a population of NK cells has at least about 55% decreased probability of survival relative to a subject who has less than 50% dysfunctional NK cells in a population of NK cells. In embodiments, a subject having 50% or more dysfunctional NK cells in a population of NK cells has at least about 60% decreased probability of survival relative to a subject who has less than 50% dysfunctional NK cells in a population of NK cells. In embodiments, a subject having 50% or more dysfunctional NK cells in a population of NK cells has at least about 65% decreased probability of survival relative to a subject who has less than 50% dysfunctional NK cells in a population of NK cells. In embodiments, a subject having 50% or more dysfunctional NK cells in a population of NK cells has at least about 70% decreased probability of survival relative to a subject who has less than 50% dysfunctional NK cells in a population of NK cells. In embodiments, a subject having 50% or more dysfunctional NK cells in a population of NK cells has at least about 75% decreased probability of survival relative to a subject who has less than 50% dysfunctional NK cells in a population of NK cells. In embodiments, a subject having 50% or more dysfunctional NK cells in a population of NK cells has at least about 80% decreased probability of survival relative to a subject who has less than 50% dysfunctional NK cells in a population of NK cells. In embodiments, a subject having 50% or more dysfunctional NK cells in a population of NK cells has at least about 85% decreased probability of survival relative to a subject who has less than 50% dysfunctional NK cells in a population of NK cells. In embodiments, a subject having 50% or more dysfunctional NK cells in a population of NK cells has at least about 90% decreased probability of survival relative to a subject who has less than 50% dysfunctional NK cells in a population of NK cells.

In embodiments, a subject having 50% or more dysfunctional NK cells in a population of NK cells has at least about 5% increased probability of relapse relative to a subject who has less than 50% dysfunctional NK cells in a population of NK cells. In embodiments, a subject having 50% or more dysfunctional NK cells in a population of NK cells has at least about 10% increased probability of relapse relative to a subject who has less than 50% dysfunctional NK cells in a population of NK cells. In embodiments, a subject having 50% or more dysfunctional NK cells in a population of NK cells has at least about 15% increased probability of relapse relative to a subject who has less than 50% dysfunctional NK cells in a population of NK cells. In embodiments, a subject having 50% or more dysfunctional NK cells in a population of NK cells has at least about 20% increased probability of relapse relative to a subject who has less than 50% dysfunctional NK cells in a population of NK cells. In embodiments, a subject having 50% or more dysfunctional NK cells in a population of NK cells has at least about 25% increased probability of relapse relative to a subject who has less than 50% dysfunctional NK cells in a population of NK cells. In embodiments, a subject having 50% or more dysfunctional NK cells in a population of NK cells has at least about 30% increased probability of relapse relative to a subject who has less than 50% dysfunctional NK cells in a population of NK cells. In embodiments, a subject having 50% or more dysfunctional NK cells in a population of NK cells has at least about 35% increased probability of relapse relative to a subject who has less than 50% dysfunctional NK cells in a population of NK cells. In embodiments, a subject having 50% or more dysfunctional NK cells in a population of NK cells has at least about 40% increased probability of relapse relative to a subject who has less than 50% dysfunctional NK cells in a population of NK cells. In embodiments, a subject having 50% or more dysfunctional NK cells in a population of NK cells has at least about 45% increased probability of relapse relative to a subject who has less than 50% dysfunctional NK cells in a population of NK cells. In embodiments, a subject having 50% or more dysfunctional NK cells in a population of NK cells has at least about 50% increased probability of relapse relative to a subject who has less than 50% dysfunctional NK cells in a population of NK cells. In embodiments, a subject having 50% or more dysfunctional NK cells in a population of NK cells has at least about 55% increased probability of relapse relative to a subject who has less than 50% dysfunctional NK cells in a population of NK cells. In embodiments, a subject having 50% or more dysfunctional NK cells in a population of NK cells has at least about 60% increased probability of relapse relative to a subject who has less than 50% dysfunctional NK cells in a population of NK cells. In embodiments, a subject having 50% or more dysfunctional NK cells in a population of NK cells has at least about 65% increased probability of relapse relative to a subject who has less than 50% dysfunctional NK cells in a population of NK cells. In embodiments, a subject having 50% or more dysfunctional NK cells in a population of NK cells has at least about 70% increased probability of relapse relative to a subject who has less than 50% dysfunctional NK cells in a population of NK cells. In embodiments, a subject having 50% or more dysfunctional NK cells in a population of NK cells has at least about 75% increased probability of relapse relative to a subject who has less than 50% dysfunctional NK cells in a population of NK cells. In embodiments, a subject having 50% or more dysfunctional NK cells in a population of NK cells has at least about 80% increased probability of relapse relative to a subject who has less than 50% dysfunctional NK cells in a population of NK cells. In embodiments, a subject having 50% or more dysfunctional NK cells in a population of NK cells has at least about 85% increased probability of relapse relative to a subject who has less than 50% dysfunctional NK cells in a population of NK cells. In embodiments, a subject having 50% or more dysfunctional NK cells in a population of NK cells has at least about 90% increased probability of relapse relative to a subject who has less than 50% dysfunctional NK cells in a population of NK cells.

In embodiments, identifying the dysfunctional NK cells includes detecting in the population of NK cells: (a) an elevated expression level of CD56 relative to a standard control; (b) an elevated expression level of CD94 relative to the standard control; (c) an elevated ratio of CD11b⁻CD27− cells to CD11b⁺CD27− cells relative to the standard control; (d) the presence of CD69; (e) the presence of a cytokine; or (f) calcium (Ca2+) mobilization; thereby identifying the dysfunctional NK cells in the subject. In embodiments, identifying the dysfunctional NK cells includes detecting in the population of NK cells: (a) an elevated expression level of CD56 relative to a standard control; (b) an elevated expression level of CD94 relative to the standard control; (c) an elevated ratio of CD11b⁻CD27− cells to CD11b⁺CD27− cells relative to the standard control; (d) the presence of CD69; (e) the presence of a cytokine; and (f) calcium (Ca2+) mobilization; thereby identifying the dysfunctional NK cells in the subject.

In embodiments, the cytokine is Interferon gamma (IFN-γ), Tumor necrosis factor (TNF), Granulocyte-macrophage colony-stimulating factor (GM-CSF), Macrophage inflammatory protein 1 beta (MIP-10), Interleukin-2 (IL-2), or combinations thereof. In embodiments, the cytokine is IFN-γ, TNF, GM-CSF, or combinations thereof.

In embodiments, the method further includes detecting the absence of DNAX Accessory Molecule-1 (DNAM 1), Killer cell immunoglobulin-like receptor 2DL1 (KIR2DL1), CD57, Siglec-7, or combinations thereof in the population of NK cells. In embodiments, the method further includes further includes detecting the presence of cytotoxic granules in said population of NK cells. In embodiments, detecting the presence of cytotoxic granules includes detecting the presence of perforin (PRF) or granzyme B (GZMB) in said population of NK cells.

In embodiments, the method includes detecting the presence of one or more activation markers in said population of NK cells. In embodiments, the activation marker is CD107a. In embodiments, the method includes detecting the presence of one or more checkpoint markers in the population of NK cells. In embodiments, the checkpoint marker is Lymphocyte-activation gene 3 (LAG-3), Killer cell lectin-like receptor subfamily G member 1 (KLRG1), Programmed cell death 1 ligand 2 (PD-L2), or combinations thereof. In embodiments, the method further includes detecting the presence of C-X-C chemokine receptor type 4 (CXCR4) in the population of NK cells.

In embodiments, the population of NK cells are enriched from bone marrow mononuclear cells (BMMC) obtained from the subject or peripheral blood mononuclear cells (PBMC) obtained from the subject. In embodiments, the leukemia is acute lymphoblastic leukemia (ALL). In embodiments, the ALL is T-cell ALL (T-ALL) or B-cell ALL (B-ALL).

In embodiments, the detecting includes a cytometric method or measuring RNA transcript levels.

In an aspect is provided a method of identifying a subject susceptible to leukemia relapse, including identifying dysfunctional natural killer (NK) cells in a population of NK cells obtained from the subject, wherein 50% or more of the population of NK cells are dysfunctional NK cells indicates that the subject is susceptible to leukemia relapse. A subject susceptible to relapse refers to a subject who is currently in remission from leukemia, and has has increased probability of relasping compared to a leukemia subject in remission who has fewer than 50% dysfunctional NK cells in a population of NK cells.

In embodiments, identifying the dysfunctional NK cells includes detecting in the population of NK cells: (a) an elevated expression level of CD56 relative to a standard control; (b) an elevated expression level of CD94 relative to the standard control; (c) an elevated ratio of CD11b⁻CD27− cells to CD11b^{m}CD27− cells relative to the standard control; (d) the presence of CD69; (e) the presence of a cytokine; or (f) calcium (Ca2+) mobilization; thereby identifying the dysfunctional NK cells in the subject. In embodiments, identifying the dysfunctional NK cells includes detecting in the population of NK cells: (a) an elevated expression level of CD56 relative to a standard control; (b) an elevated expression level of CD94 relative to the standard control; (c) an elevated ratio of CD11b⁻CD27− cells to CD11b⁺CD27− cells relative to the standard control; (d) the presence of CD69; (e) the presence of a cytokine; and (f) calcium (Ca2+) mobilization; thereby identifying the dysfunctional NK cells in the subject.

In embodiments, the cytokine is Interferon gamma (IFN-γ), Tumor necrosis factor (TNF), Granulocyte-macrophage colony-stimulating factor (GM-CSF), Macrophage inflammatory protein 1 beta (MIP-10), Interleukin-2 (IL-2), or combinations thereof. In embodiments, the cytokine is IFN-γ, TNF, GM-CSF, or combinations thereof.

In embodiments, the method further includes detecting the absence of DNAX Accessory Molecule-1 (DNAM 1), Killer cell immunoglobulin-like receptor 2DL1 (KIR2DL1), CD57, Siglec-7, or combinations thereof in the population of NK cells. In embodiments, the method further includes further includes detecting the presence of cytotoxic granules in said population of NK cells. In embodiments, detecting the presence of cytotoxic granules includes detecting the presence of perforin (PRF) or granzyme B (GZMB) in said population of NK cells.

In embodiments, the method includes detecting the presence of one or more activation markers in said population of NK cells. In embodiments, the activation marker is CD107a. In embodiments, the method includes detecting the presence of one or more checkpoint markers in the population of NK cells. In embodiments, the checkpoint marker is Lymphocyte-activation gene 3 (LAG-3), Killer cell lectin-like receptor subfamily G member 1 (KLRG1), Programmed cell death 1 ligand 2 (PD-L2), or combinations thereof. In embodiments, the method further includes detecting the presence of C-X-C chemokine receptor type 4 (CXCR4) in the population of NK cells.

In embodiments, the population of NK cells are enriched from bone marrow mononuclear cells (BMMC) obtained from the subject or peripheral blood mononuclear cells (PBMC) obtained from the subject. In embodiments, the leukemia is acute lymphoblastic leukemia (ALL).

In embodiments, the detecting includes a cytometric method or measuring RNA transcript levels.

The methods provided herein are contemplated to be useful for treating subject with leukemia who have previously been treated for leukemia, such as with induction therapy. In embodiments, the elevated presence of a cytokine compared to a control indicates that the NK cell is a dysfunctional NK cell. In embodiments, the cytokine is IFN-γ, TNF, or GM-CSF. In embodiments, the elevated presence of CD56 compared to a control indicates that the NK cell is a dysfunctional NK cell. In embodiments, the elevated presence of CD69 compared to a control indicates that the NK cell is a dysfunctional NK cell. In embodiments, the elevated presence of CD94 compared to a control indicates that the NK cell is a dysfunctional NK cell. In embodiments, the elevated ratio of CD11b⁻CD27− cells to CD11b⁺CD27− cells relative to a standard control indicates that the NK cell is a dysfunctional NK cell. In embodiments, the elevated level of two or more of the above markers and/or cytokines indicates that the NK cell is a dysfunctional NK cell.

In embodiments of the methods provided herein, only the markers disclosed herein are detected and no other markers are detected. In embodiments, only CD56, CD94, ratio of CD11b⁻CD27− NK cells to CD11b⁺CD27− NK cells, CD69, a cytokine (IFN-γ, TNF, GM-CSF, MIP-10, IL-2); Ca²⁺ mobilization, DNAM 1, KIR2DL1, CD57, Siglec-7, PRF, GZMB, CD107a, LAG-3, KLRG1, PD-L2, and CXCR4 are detected and no other markers are detected. In embodiments, only CD56 and one or more of: CD94, ratio of CD11b⁻CD27− NK cells to CD11b⁺CD27− NK cells, CD69, a cytokine (IFN-γ, TNF, GM-CSF, MIP-10, IL-2); Ca²⁺ mobilization, DNAM 1, KIR2DL1, CD57, Siglec-7, PRF, GZMB, CD107a, LAG-3, KLRG1, PD-L2, and CXCR4 are detected and no other markers are detected.

### EMBODIMENTS

P Embodiment 1. A method of detecting dysfunctional NK cells in a leukemia patient, the method comprising detecting in a plurality of NK cells in a biological sample obtained from a leukemia patient: (a) an elevated level of a cytokine relative to a standard control; (b) an elevated level of a CD56 relative to a standard control; (c) an elevated level of CD69 relative to a standard control; (d) an elevated level of CD94 relative to a standard control; (e) an elevated ratio of CD11b⁻CD27− cells to CD11b⁺CD27− cells relative to a standard control; or, (f) an elevated level of calcium (Ca2+) mobilization relative to a standard control, thereby detecting dysfunctional NK cells in said leukemia patient.

P Embodiment 2. The method of P embodiment 1, wherein the cytokine is IFN-γ, TNF, or GM-CSF.

P Embodiment 3. The method of P embodiment 1 or 2 wherein the leukemia is acute lymphoblastic leukemia (ALL).

P Embodiment 4. A method of treating leukemia in a subject in need thereof, the method comprising administering to said subject an effective amount of allogeneic NK cells when the NK cell population in the patient is >50% dysfunctional NK cells.

P Embodiment 5. The method of P embodiment 4, further comprising detecting in a population of NK cells in a biological sample obtained from said subject: (a) an elevated level of a cytokine relative to a standard control; (b) an elevated level of a CD56 relative to a standard control; (c) an elevated level of CD69 relative to a standard control; (d) an elevated level of CD94 relative to a standard control; (e) an elevated ratio of CD11b⁻CD27− cells to CD11b⁺CD27− cells relative to a standard control; or, (f) an elevated level of calcium (Ca2+) mobilization relative to a standard control, thereby detecting dysfunctional NK cells in said leukemia patient.

P Embodiment 6. The method of P embodiment 5, wherein the cytokine is IFN-γ, TNF, or GM-CSF.

P Embodiment 7. The method of P embodiment 5 or 6, wherein the subject was previously treated for leukemia.

P Embodiment 8. The method of P embodiment 7, wherein the subject was non-responsive or relapsed from the previous treatment.

P Embodiment 9. The method of any of P embodiments 5-7, wherein the leukemia is acute lymphoblastic leukemia (ALL).

P Embodiment 10. A method of treating leukemia in a subject in need thereof, the method comprising: (a) detecting>50% dysfunctional NK cells in a biological sample obtained from said subject; and (b) administering to said subject an effective amount of allogeneic NK cells.

P Embodiment 11. The method of P embodiment 10, further comprising detecting in a population of NK cells in a biological sample obtained from said subject: (a) an elevated level of a cytokine relative to a standard control; (b) an elevated level of a CD56 relative to a standard control; (c) an elevated level of CD69 relative to a standard control; (d) an elevated level of CD94 relative to a standard control; (e) an elevated ratio of CD11b⁻CD27− cells to CD11b⁺CD27− cells relative to a standard control; or, (f) an elevated level of calcium (Ca2+) mobilization relative to a standard control, thereby detecting dysfunctional NK cells in said leukemia patient.

P Embodiment 12. The method of P embodiment 11, wherein the cytokine is IFN-γ, TNF, or GM-CSF.

P Embodiment 13. The method of P embodiment 11 or 12, wherein the subject was previously treated for leukemia.

P Embodiment 14. The method of P embodiment 13, wherein the subject was non-responsive or relapsed from the previous treatment.

P Embodiment 15. The method of any of P embodiments 10-14, wherein the leukemia is acute lymphoblastic leukemia (ALL).

P Embodiment 16. A method of determining the probability of survival or relapse in a leukemia patient, the method comprising determining the proportion of dysfunctional NK cells in a biological sample obtained from the leukemia patient, wherein >50% dysfunctional NK cells in the biological sample indicates that the patient has a decreased probability of survival or an increased probability of relapse.

### Embodiments

Embodiment 1. A method of identifying dysfunctional natural killer (NK) cells in a population of NK cells obtained from a subject having leukemia, wherein the method comprises detecting in said population of NK cells: (a) an elevated expression level of CD56 relative to a standard control; (b) an elevated expression level of CD94 relative to the standard control; (c) an elevated ratio of CD11b⁻CD27− NK cells to CD11b⁺CD27− NK cells relative to the standard control; (d) the presence of CD69; (e) the presence of a cytokine; or (f) calcium (Ca²¹) mobilization; thereby identifying the dysfunctional NK cells.

Embodiment 2. The method of embodiment 1, wherein the cytokine is Interferon gamma (IFN-γ), Tumor necrosis factor (TNF), Granulocyte-macrophage colony-stimulating factor (GM-CSF), Macrophage inflammatory protein 1 beta (MIP-10), Interleukin-2 (IL-2), or combinations thereof.

Embodiment 3. The method of embodiment 1 or 2, wherein the cytokine is IFN-γ, TNF, GM-CSF, or combinations thereof.

Embodiment 4. The method of any one of embodiments 1-3, further comprising detecting the absence of DNAX Accessory Molecule-1 (DNAM 1), Killer cell immunoglobulin-like receptor 2DL1 (KIR2DL1), CD57, Siglec-7, or combinations thereof in said population of NK cells.

Embodiment 5. The method of any one of embodiments 1-4, further comprising detecting the presence of cytotoxic granules in said population of NK cells.

Embodiment 6. The method of embodiment 5, wherein detecting the presence of cytotoxic granules comprises detecting the presence of perforin (PRF) or granzyme B (GZMB) in said population of NK cells.

Embodiment 7. The method of any one of embodiments 1-6, further comprising detecting the presence of one or more activation markers in said population of NK cells.

Embodiment 8. The method of embodiment 7, wherein the activation marker is CD107a.

Embodiment 9. The method of any one of embodiments 1-8, further comprising detecting the presence of one or more checkpoint markers in said population of NK cells.

Embodiment 10. The method of embodiment 9, wherein the checkpoint marker is Lymphocyte-activation gene 3 (LAG-3), Killer cell lectin-like receptor subfamily G member 1 (KLRG1), Programmed cell death 1 ligand 2 (PD-L2), or combinations thereof.

Embodiment 11. The method of any one of embodiments 1-10, further comprising detecting the presence of C-X-C chemokine receptor type 4 (CXCR4) in said population of NK cells.

Embodiment 12. The method of any one of embodiments 1-11, wherein the population of NK cells are enriched from bone marrow mononuclear cells (BMMC) obtained from said subject or peripheral blood mononuclear cells (PBMC) obtained from said subject.

Embodiment 13. The method of any one of embodiments 1-12, wherein the leukemia is acute lymphoblastic leukemia (ALL).

Embodiment 14. The method of embodiment 13, wherein the ALL is T-cell ALL (T-ALL) or B-cell ALL (B-ALL).

Embodiment 15. The method of any one of embodiments 1-14, wherein the detecting comprises a cytometric method or measuring RNA transcript levels.

Embodiment 16. The method of any one of embodiments 1-14, wherein the detecting comprises a cytometric method.

Embodiment 17. The method of any one of embodiments 1-16, wherein 50% or more of the population of NK cells are dysfunctional NK cells.

Embodiment 18. The method of embodiment 17, further comprising administering to the subject an effective amount of allogeneic NK cells.

Embodiment 19. The method of embodiment 18, wherein the allogeneic NK cells are obtained from a subject who does not have leukemia.

Embodiment 20. The method of embodiment 18 or 19, wherein the allogeneic NK cells are not dysfunctional NK cells.

Embodiment 21. A method of treating leukemia in a subject in need thereof, comprising administering to the subject an effective amount of allogeneic NK cells, wherein 50% or more of a population of NK cells obtained from the subject are dysfunctional NK cells.

Embodiment 22. The method of embodiment 21, comprising obtaining the population of NK cells from the subject and identifying the dysfunctional NK cells prior to administering the effective amount of allogeneic NK cells.

Embodiment 23. The method of embodiment 22, wherein identifying the dysfunctional NK cells comprising detecting in said population of NK cells: (a) an elevated expression level of CD56 relative to a standard control; (b) an elevated expression level of CD94 relative to the standard control; (c) an elevated ratio of CD11b⁻CD27− NK cells to CD11b⁺CD27− NK cells relative to the standard control; (d) the presence of CD69; (e) the presence of a cytokine; or (f) calcium (Ca²¹) mobilization.

Embodiment 24. The method of embodiment 23, wherein the cytokine is Interferon gamma (IFN-γ), Tumor necrosis factor (TNF), Granulocyte-macrophage colony-stimulating factor (GM-CSF), Macrophage inflammatory protein 1 beta (MIP-10), Interleukin-2 (IL-2), or combinations thereof.

Embodiment 25. The method of embodiment 23 or 24, wherein the cytokine is IFN-7, TNF, GM-CSF, or combinations thereof.

Embodiment 26. The method of any one of embodiments 23-25, further comprising detecting the absence of DNAX Accessory Molecule-1 (DNAM 1), Killer cell immunoglobulin-like receptor 2DL1 (KIR2DL1), CD57, Siglec-7, or combinations thereof in said population of NK cells.

Embodiment 27. The method of any one of embodiments 23-26, further comprising detecting the presence of cytotoxic granules in said population of NK cells.

Embodiment 28. The method of embodiment 27, wherein detecting the presence of cytotoxic granules comprises detecting the presence of perforin (PRF) or granzyme B (GZMB) in said population of NK cells.

Embodiment 29. The method of any one of embodiments 23-28, further comprising detecting the presence of one or more activation markers in said population of NK cells.

Embodiment 30. The method of embodiment 29, wherein the activation marker is CD107a.

Embodiment 31. The method of any one of embodiments 23-30, further comprising detecting the presence of one or more checkpoint markers in said population of NK cells.

Embodiment 32. The method of embodiment 31, wherein the checkpoint marker is Lymphocyte-activation gene 3 (LAG-3), Killer cell lectin-like receptor subfamily G member 1 (KLRG1), Programmed cell death 1 ligand 2 (PD-L2), or combinations thereof.

Embodiment 33. The method of any one of embodiments 23-32, further comprising detecting the presence of C-X-C chemokine receptor type 4 (CXCR4) in said population of NK cells.

Embodiment 34. The method of any one of embodiments 21-33, wherein the population of NK cells are enriched from bone marrow mononuclear cells (BMMC) obtained from said subject or peripheral blood mononuclear cells (PBMC) obtained from said subject.

Embodiment 35. The method of any one of embodiments 21-34, wherein the leukemia is acute lymphoblastic leukemia (ALL).

Embodiment 36. The method of any one of embodiments 21-36, wherein the detecting comprises a cytometric method or measuring RNA transcript levels.

Embodiment 37. The method of any one of embodiments 21-36, wherein the subject previously received treatment for leukemia.

Embodiment 38. The method of embodiment 37, wherein the subject was non-responsive to the previous treatment.

Embodiment 39. The method of embodiment 37, wherein the subject has relapsed.

Embodiment 40. The method of any one of embodiments 21-39, wherein the allogeneic NK cells are obtained from a subject who does not have leukemia.

Embodiment 41. The method of any one of embodiments 21-40, wherein the allogeneic NK cells are not dysfunctional NK cells.

Embodiment 42. A method of treating leukemia in a subject in need thereof, comprising: a) identifying dysfunctional natural killer (NK) cells in a population of NK cells obtained from the subject; and b) administering to the subject an effective amount of allogeneic NK cells; wherein 50% or more of the population of NK cells are dysfunctional NK cells.

Embodiment 43. The method of embodiment 42, wherein identifying the dysfunctional natural killer (NK) cells comprises detecting in said population of NK cells: (a) an elevated expression level of CD56 relative to a standard control; (b) an elevated expression level of CD94 relative to the standard control; (c) an elevated ratio of CD11b-CD27− NK cells to CD11b⁺CD27− NK cells relative to the standard control; (d) the presence of CD69; (e) the presence of a cytokine; or (f) calcium (Ca²¹) mobilization; thereby identifying the dysfunctional NK cells.

Embodiment 44. The method of embodiment 43, wherein the cytokine is Interferon gamma (IFN-γ), Tumor necrosis factor (TNF), Granulocyte-macrophage colony-stimulating factor (GM-CSF), Macrophage inflammatory protein 1 beta (MIP-10), Interleukin-2 (IL-2), or combinations thereof.

Embodiment 45. The method of embodiment 43 or 44, wherein the cytokine is IFN-7, TNF, GM-CSF, or combinations thereof.

Embodiment 46. The method of any one of embodiments 43-45, further comprising detecting the absence of DNAX Accessory Molecule-1 (DNAM 1), Killer cell immunoglobulin-like receptor 2DL1 (KIR2DL1), CD57, Siglec-7, or combinations thereof in said population of NK cells.

Embodiment 47. The method of any one of embodiments 43-46, further comprising detecting the presence of cytotoxic granules in said population of NK cells.

Embodiment 48. The method of any one of embodiments 47, wherein detecting the presence of cytotoxic granules comprises detecting the presence of perforin (PRF) or granzyme B (GZMB) in said population of NK cells.

Embodiment 49. The method of any one of embodiments 43-48, further comprising detecting the presence of one or more activation markers in said population of NK cells.

Embodiment 50. The method of embodiment 49, wherein the activation marker is CD107a.

Embodiment 51. The method of any one of embodiments 43-50, further comprising detecting the presence of one or more checkpoint markers in said population of NK cells in said population of NK cells.

Embodiment 52. The method of embodiment 51, wherein the checkpoint marker is Lymphocyte-activation gene 3 (LAG-3), Killer cell lectin-like receptor subfamily G member 1 (KLRG1), Programmed cell death 1 ligand 2 (PD-L2), or combinations thereof.

Embodiment 53. The method of any one of embodiments 43-52, further comprising detecting the presence of C-X-C chemokine receptor type 4 (CXCR4) in said population of NK cells.

Embodiment 54. The method of any one of embodiments 42-53, wherein the population of NK cells are enriched from bone marrow mononuclear cells (BMMC) obtained from said subject or peripheral blood mononuclear cells (PBMC) obtained from said subject.

Embodiment 55. The method of any one of embodiments 42-54, wherein the subject was previously treated for leukemia.

Embodiment 56. The method of embodiment 55, wherein the subject was non-responsive or has relapsed.

Embodiment 57. The method of any of embodiments 52-56, wherein the leukemia is acute lymphoblastic leukemia (ALL).

Embodiment 58. The method of any one of embodiments 42-57, wherein the allogeneic NK cells are obtained from a subject who does not have leukemia.

Embodiment 59. The method of any one of embodiments 42-58, wherein the allogeneic NK cells are not dysfunctional NK cells.

Embodiment 60. A method of determining a probablility of survival or relapse in a subject having leukemia, comprising identifying dysfunctional natural killer (NK) cells in a population of NK cells obtained from the subject, wherein 50% or more of the population of NK cells are dysfunctional NK cells indicates that the subject has decreased probability of survival or increased probability of relapse relative to a subject wherein less than 50% of the population of NK cells are dysfunctional NK cells.

Embodiment 61. The method of embodiment 60, wherein identifying dysfunctional NK cells comprises detecting in said population of NK cells: A method of identifying dysfunctional natural killer (NK) cells in a population of NK cells obtained from a subject having leukemia, wherein the method comprises detecting in said population of NK cells: (a) an elevated expression level of CD56 relative to a standard control; (b) an elevated expression level of CD94 relative to the standard control; (c) an elevated ratio of CD11b-CD27− NK cells to CD11b⁺CD27− NK cells relative to the standard control; (d) the presence of CD69; (e) the presence of a cytokine; or (f) calcium (Ca²¹) mobilization.

Embodiment 62. The method of embodiment 61, wherein the cytokine is Interferon gamma (IFN-γ), Tumor necrosis factor (TNF), Granulocyte-macrophage colony-stimulating factor (GM-CSF), Macrophage inflammatory protein 1 beta (MIP-10), Interleukin-2 (IL-2), or combinations thereof.

Embodiment 63. The method of embodiment 61 or 62, wherein the cytokine is IFN-7, TNF, GM-CSF, or combinations thereof.

Embodiment 64. The method of any one of embodiments 61-63, further comprising detecting the absence of DNAX Accessory Molecule-1 (DNAM 1), Killer cell immunoglobulin-like receptor 2DL1 (KIR2DL1), CD57, Siglec-7, or combinations thereof in said population of NK cells.

Embodiment 65. The method of any one of embodiments 61-64, further comprising detecting the presence of cytotoxic granules in said population of NK cells.

Embodiment 66. The method of embodiment 65, wherein detecting the presence of cytotoxic granules comprises detecting the presence of perforin (PRF) or granzyme B (GZMB) in said population of NK cells.

Embodiment 67. The method of any one of embodiments 61-66, further comprising detecting the presence of one or more activation markers in said population of NK cells.

Embodiment 68. The method of embodiment 67, wherein the activation marker is CD107a.

Embodiment 69. The method of any one of embodiments 61-68, further comprising detecting the presence of one or more checkpoint markers in said population of NK cells.

Embodiment 70. The method of embodiment 69, wherein the checkpoint marker is Lymphocyte-activation gene 3 (LAG-3), Killer cell lectin-like receptor subfamily G member 1 (KLRG1), Programmed cell death 1 ligand 2 (PD-L2), or combinations thereof.

Embodiment 71. The method of any one of embodiments 61-70, further comprising detecting the presence of C-X-C chemokine receptor type 4 (CXCR4) in said population of NK cells.

Embodiment 72. The method of any one of embodiments 60-71, wherein the population of NK cells are enriched from bone marrow mononuclear cells (BMMC) obtained from said subject or peripheral blood mononuclear cells (PBMC) obtained from said subject.

Embodiment 73. The method of any one of embodiments 60-72, wherein the leukemia is acute lymphoblastic leukemia (ALL).

Embodiment 74. The method of any one of embodiments 61-73, wherein the detecting is a cytometric method or measuring RNA transcript levels.

Embodiment 75. A method of identifying a subject susceptible to leukemia relapse, comprising identifying dysfunctional natural killer (NK) cells in a population of NK cells obtained from the subject, wherein 50% or more of the population of NK cells are dysfunctional NK cells indicates that the subject is susceptible to leukemia relapse.

Embodiment 76. The method of embodiment 75, wherein identifying dysfunctional NK cells comprises detecting in said population of NK cells: (a) an elevated expression level of CD56 relative to a standard control; (b) an elevated expression level of CD94 relative to the standard control; (c) an elevated ratio of CD11b⁻CD27− NK cells to CD11b⁺CD27− NK cells relative to the standard control; (d) the presence of CD69; (e) the presence of a cytokine; or (f) calcium (Ca²¹) mobilization.

Embodiment 77. The method of embodiment 76, wherein the cytokine is Interferon gamma (IFN-γ), Tumor necrosis factor (TNF), Granulocyte-macrophage colony-stimulating factor (GM-CSF), Macrophage inflammatory protein 1 beta (MIP-10), Interleukin-2 (IL-2), or combinations thereof.

Embodiment 78. The method of embodiment 76 or 77, wherein the cytokine is IFN-7, TNF, GM-CSF, or combinations thereof.

Embodiment 79. The method of any one of embodiments 76-78, further comprising detecting the absence of DNAX Accessory Molecule-1 (DNAM 1), Killer cell immunoglobulin-like receptor 2DL1 (KIR2DL1), CD57, Siglec-7, or combinations thereof in said population of NK cells.

Embodiment 80. The method of any one of embodiments 76-79, further comprising detecting the presence of cytotoxic granules in said population of NK cells.

Embodiment 81. The method of embodiment 80, wherein detecting the presence of cytotoxic granules comprises detecting the presence of perforin (PRF) or granzyme B (GZMB) in said population of NK cells.

Embodiment 82. The method of any one of embodiments 76-81, further comprising detecting the presence of one or more activation markers in said population of NK cells.

Embodiment 83. The method of embodiment 82, wherein the activation marker is CD107a.

Embodiment 84. The method of any one of embodiments 76-83, further comprising detecting the presence of one or more checkpoint markers in said population of NK cells.

Embodiment 85. The method of embodiment 84, wherein the checkpoint marker is Lymphocyte-activation gene 3 (LAG-3), Killer cell lectin-like receptor subfamily G member 1 (KLRG1), Programmed cell death 1 ligand 2 (PD-L2), or combinations thereof.

Embodiment 86. The method of any one of embodiments 76-85, further comprising detecting the presence of C-X-C chemokine receptor type 4 (CXCR4) in said population of NK cells.

Embodiment 87. The method of any one of embodiments 76-86, wherein the population of NK cells are enriched from bone marrow mononuclear cells (BMMC) obtained from said subject or peripheral blood mononuclear cells (PBMC) obtained from said subject.

Embodiment 88. The method of any one of embodiments 75-87, wherein the leukemia is acute lymphoblastic leukemia (ALL).

Embodiment 89. The method of any one of embodiments 75-88, wherein the detecting comprises a cytometric method or measuring RNA transcript levels.

### EXAMPLES

### Example 1: NK Cell Treatment Therapies

As effector cytotoxic lymphocytes, NK cells are attractive immune cell-based therapy candidates. NK cells have been explored for treatment of acute myeloid leukemia (AML) and ALL even prior to CAR-T cells. NK cells have advantages over CAR-T cells: (1) Development of allogeneic CAR-T cell immunotherapies is complex due to required disruption of mechanisms that cause graft-versus-host disease in recipients. However, NK cells, being less haplotype restricted, can be developed as ‘off-the-shelf’ immunotherapies. (2) Treating T-ALL using CAR-T cells is difficult because of reduced healthy T-lymphocytes in patients and on-target effects on normal T cell. (3) B-ALL may become resistant to CAR-T cells by losing target antigen expression. (4) Engineering autologous CAR-T cells to treat infant B-ALL can be challenging because of difficulties in obtaining peripheral blood from these patients. (5) Reduced cytokine release syndrome and neurotoxicity make NK cells safer than CAR-T cells. Therefore, NK therapies are an attractive potential treatment for ALL.

### Example 2: Identification of Dysfunctional NK Cells

### Methods

### Patient Samples

Bone marrow (BMMC) and peripheral blood mononuclear cells (PBMC) were collected and processed from consented B/T-ALL patients and healthy donors according to Institutional Review Board (IRB) policies. Healthy BMMC were purchased from AllCells, California. Healthy PBMC were isolated from buffy coats procured from Stanford Blood Center, City of Hope Michael Amini Transfusion Medicine Center, and influenza vaccine studies⁵¹ of Stanford Biobank. Deidentified B/T-ALL patient specimens were used.

### CyTOF

Samples were processed as previously described. After thawing, cells were divided in 2 samples: ‘unstimulated’ and ‘stimulated’. After overnight rest, ‘stimulated’ cells were incubated with PMA/ionomycin; during incubation, anti-CD107a, Brefeldin A and Monensin were added in all samples. Dead cells were identified using Cell-ID™ Cisplatin-195Pt (Fluidigm) prior to surface staining, then fixed with 2% PFA, followed by intracellular and DNA-staining with CELL-ID™ Intercalator-Ir (Fluidigm) (antibodies in Tables 1-2). Prior to acquisition (CyTOF Helios, Fluidigm), cells were washed with Milli-Q water and resuspended in 1× solution of EQ™ Four Element Calibration Beads (Fluidigm). Data were normalized using MATLAB normalizer prior analysis with Cytobank.

TABLE 1
Antibodies used for CyTOF immunophenotyping (Panel 2)

| | | | | Concen- | |
|---|---|---|---|---|---|
| Metal | | | | tration | Titre |
| label | Target | Clone | Source | (μg/mL) | (μg/mL) |
| 113In | CD57 | HNK1 | Custom, | 265 | 2.5 |
| | | | Biolegend | | |
| 141Pr | HLA-DR | L243 | Custom, | 425 | 2 |
| | | | Biolegend | | |
| 142Nd | CD19 | HIB19 | Fluidigm | 500 | 5 |
| 145Nd | CD4 | RPA-T4 | Fluidigm | 500 | 5 |
| 146Nd | CD8 | RPA-T8 | Fluidigm | 500 | 5 |
| 147Sm | CD20 | 2H7 | Fluidigm | 500 | 5 |
| 149Sm | CTLA-4 | 14D3 | Custom, | 510 | 5 |
| | | | eBioscience | | |
| 150Nd | MIP-1β | D21-1351 | Fluidigm | 500 | 5 |
| 151Eu | CD107a | H4A3 | Fluidigm | 500 | 5 |
| 152Sm | TNF-α | Mab11 | Fluidigm | 500 | 5 |
| 153Eu | CD45RA | HI100 | Fluidigm | 500 | 5 |
| 154Sm | CD3 | UCHT1 | Fluidigm | 500 | 5 |
| 158Gd | CD33 | WM53 | Fluidigm | 500 | 5 |
| 159Tb | GM-CSF | BVD2-21C11 | Fluidigm | 500 | 5 |
| 160Gd | CD14 | M5E2 | Fluidigm | 500 | 5 |
| 161Dy | IFN-γ | 4S.B3 | Custom, | 660 | 2.5 |
| | | | Biolegend | | |
| 162Dy | NKp46 | BAB281 | Fluidigm | 500 | 5 |
| 166Er | IL-2 | MQ1-17h12 | Fluidigm | 500 | 5 |
| 167Er | CD27 | L128 | Fluidigm | 500 | 5 |
| 170Er | PD1 | EH12.1, | Fluidigm | 500 | 2.5 |
| | | BD | | | |
| 171Yb | Granzyme | GB11 | Fluidigm | 500 | 5 |
| | B | | | | |
| 172Yb | PD-L2 | 24F.10C12 | Fluidigm | 500 | 5 |
| 173Yb | Perforin | B-D48 | Custom, | 500 | 2.5 |
| | | | Abcam | | |
| 175Lu | PD-L1 | 29E.2A3 | Fluidigm | 500 | 5 |
| 176Yb | CD56 | NCAM16.2 | Fluidigm | 500 | 5 |
| 209Bi | CD16 | 3G8 | Fluidigm | 500 | 5 |

TABLE 2
Antibodies used for CyTOF immunophenotyping (Panel 3)

| | | | | Concen- | |
|---|---|---|---|---|---|
| Metal | | | | tration | Titre |
| label | Target | Clone | Source | (μg/mL) | (μg/mL) |
| 113In | CD57 | HNK1 | Custom, | 265 | 2.5 |
| | | | Biolegend | | |
| 141Pr | HLA-DR | L243 | Custom, | 425 | 2 |
| | | | Biolegend | | |
| 142Nd | CD19 | HIB19 | Fluidigm | 500 | 5 |
| 145Nd | CD4 | RPA-T4 | Fluidigm | 500 | 5 |
| 146Nd | CD8 | RPA-T8 | Fluidigm | 500 | 5 |
| 147Sm | CD20 | 2H7 | Fluidigm | 500 | 5 |
| 148Nd | ICOS | C398.4A | Fluidigm | 500 | 5 |
| 149Sm | CTLA-4 | 14D3 | Custom, | 510 | 5 |
| | | | eBioscience | | |
| 152Sm | TNF-α | Mab11 | Fluidigm | 500 | 5 |
| 153Eu | TIGIT | MBSA43 | Fluidigm | 500 | 5 |
| 154Sm | CD3 | UCHT1 | Fluidigm | 500 | 5 |
| 157Gd | ILT2 | GHI/75 | Custom, | 460 | 5 |
| | | | Biolengend | | |
| 158Gd | CD33 | WM53 | Fluidigm | 500 | 5 |
| 159Tb | GM-CSF | BVD2-21C11 | Fluidigm | 500 | 5 |
| 160Gd | CD14 | M5E2 | Fluidigm | 500 | 5 |
| 161Dy | IFN-γ | 4S.B3 | Custom, | 660 | 2.5 |
| | | | Biolegend | | |
| 162Dy | NKp46 | BAB281 | Fluidigm | 500 | 5 |
| 163Dy | Siglec-7 | 6-434 | Custom, | 215 | 2 |
| | | | Biolegend | | |
| 164Dy | KLRG1 | SA231A2 | Custom, | 445 | 2 |
| | | | Biolegend | | |
| 165Ho | LAG-3 | 11C3C65 | Fluidigm | 500 | 5 |
| 167Er | CD27 | L128 | Fluidigm | 500 | 5 |
| 168Er | Ki-67 | B56 | Fluidigm | 500 | 5 |
| 169Tm | TIM-3 | F38-2E2 | Fluidigm | 500 | 5 |
| 170Er | PD1 | EH12.1, | Fluidigm | 500 | 2.5 |
| | | BD | | | |
| 172Yb | PD-L2 | 24F.10C12 | Fluidigm | 500 | 5 |
| 175Lu | PD-L1 | 29E.2A3 | Fluidigm | 500 | 5 |
| 176Yb | CD56 | NCAM16.2 | Fluidigm | 500 | 5 |
| 209Bi | CD16 | 3G8 | Fluidigm | 500 | 5 |

### NK Isolation and Cytotoxicity

CD56⁺ cells were enriched using Release CD56-microbeads (Miltenyi) followed by staining with anti-CD3, anti-CD56, and DAPI (Table 3). DAPI-CD3-CD56⁺ NK cells were sorted using BD FACSAria Fusion cytometer and used for cytotoxicity assays. Targets were labelled with 2.5 μM CFSE-violet dye (CellTrace-violet cell proliferation kit, Invitrogen) and co-cultured with NK cells at Effector:Target=10:1 in complete RPMI media (10% FBS, 100 U/mL Penicillin, 100 g/mL Streptomycin). After 5 h, cells were stained with 7-AAD and NK-mediated cytotoxicity was measured on BD Fortessa X20 cytometer. Data were analyzed using FlowJo10.7.1. Specific cytotoxicity=[(7-AAD⁺ target-cell frequency in coculture with effector cells—7-AAD⁺ target-cell frequency alone)/(100−7-AAD⁺ target-cell frequency alone)]×100.

TABLE 3
Reagents and antibodies used for flow cytometry

| | | | Dilution/ | |
|---|---|---|---|---|
| | | | Concen- | |
| Name | Fluorophore | Clone | tration | Source |
| Anti-human | FITC | UCHT1 | 1:100 | Biolegend |
| CD3 | | | | |
| Anti-human | APC | 5.1H11 | 1:100 | Biolegend |
| CD56 | | | | |
| Anti-human | PerCP/FITC | 2D1 | 1:100 | Biolegend |
| CD45 | | | | |
| Anti-human | PECY7 | DX22 | 1:100 | Biolegend |
| CD94 | | | | |
| Anti-human | PE | P44-8 | 1:100 | Biolegend |
| NKp44 | | | | |
| Anti-human | PECY5 | DREG-56 | 1:100 | Biolegend |
| CD62L | | | | |
| Anti-human | BV605 | 11A8 | 1:100 | Biolegend |
| DNAM-1 | | | | |
| Anti-human | APC-R700 | M1/70 | 1:100 | BD |
| CD11b | | | | |
| Anti-human | PE | M-T271 | 3:100 | BD |
| CD27 | | | | |
| Anti-human | BV711 | 3G8 | 1:100 | BD |
| CD16 | | | | |
| Anti-human | BV421 | HP-3E4 | 1:100 | BD |
| CD158a | | | | |
| Anti-human | BV605 | 131411 | 1:100 | BD |
| NKG2A | | | | |
| Anti-human | BV750 | P30-15 | 1:100 | BD |
| NKp30 | | | | |
| Anti-human | BV510 | NCAM16.2 | 1:100 | BD |
| CD56 | | | | |
| Anti-human | BUV805 | UCHT1 | 1:100 | BD |
| CD3 | | | | |
| Anti-human | BUV737 | FN50 | 1:100 | BD |
| CD69 | | | | |
| Anti-human | BUV661 | HIB19 | 1:100 | BD |
| CD19 | | | | |
| Anti-human | BUV395 | M5E2 | 1:100 | BD |
| CD14 | | | | |
| Ghost-Dye | NA | NA | 1:100 | Tonbo |
| UV450 | | | | Biosciences |
| Indo-AM | NA | NA | 1.5 μM | ThermoFisher |
| | | | | Scientific |
| 7AAD | NA | NA | 1:100 | Biolegend |
| DAPI | NA | NA | 300 nM | Biolegend |

### Flow Cytometry

PBMCs were thawed in complete RPMI containing Pierce Universal Nuclease (25U/mL, ThermoFisher Scientific). Cells were stained with fluorochrome-tagged surface antibodies (Table 3) and Ghost-UV450 for 30 min on ice. Cells were fixed with 1% PFA (Biolegend) followed by data acquisition on BD FACSymphony cytometer. FCS files were analyzed using FlowJo10.7.1. Table 4 shows analyzed NK cell numbers per patient.

TABLE 4
Number of NK cell events acquired by CyTOF and flow cytometry (FCM)

| | | | | Unstimulated | Stimulated |
|---|---|---|---|---|---|
| | | ALL | | NK cell | NK cell |
| Sample Name | Tissue | subtype | Assay | events | events |
| 128 | BMMC | B-ALL | CyTOF | 4346 | 3512 |
| 227 | BMMC | B-ALL | CyTOF | 2899 | 2571 |
| 268 | BMMC | B-ALL | CyTOF | 327 | 328 |
| 367 | BMMC | B-ALL | CyTOF | 10242 | 10918 |
| 393 | BMMC | B-ALL | CyTOF | 1847 | 1733 |
| 718 | BMMC | Burkitt's | CyTOF | 989 | 855 |
| | | B cell | | | |
| | | lymphoma | | | |
| 6457 | BMMC | B-ALL | CyTOF | 289 | 282 |
| 18067-HTB19-222 | BMMC | B-ALL | CyTOF | 852 | 2284 |
| 334 | BMMC | T-ALL | CyTOF | 15189 | 11964 |
| 552 | BMMC | T-ALL | CyTOF | 4016 | 4667 |
| 3277 | BMMC | T-ALL | CyTOF | 10001 | 9702 |
| 6043 | BMMC | T-ALL | CyTOF | 1783 | 1463 |
| 6487 | BMMC | T-ALL | CyTOF | 31503 | 36981 |
| 65 | PBMC | B-ALL | CyTOF | 625 | 540 |
| 779 | PBMC | B-ALL | CyTOF | 1511 | 1461 |
| 810 | PBMC | B-ALL | CyTOF | 1356 | 6584 |
| 2142 | PBMC | B-ALL | CyTOF | 3760 | 3149 |
| 3113 | PBMC | B-ALL | CyTOF | 3577 | 3167 |
| 4986 | PBMC | B-ALL | CyTOF | 2150 | 2150 |
| 4988 | PBMC | B-ALL | CyTOF | 2315 | 2249 |
| 18067-LTB18-544 | PBMC | B-ALL | CyTOF | 1500 | 1855 |
| 5921 | PBMC | T-ALL | CyTOF | 2513 | 2812 |
| 6070 | PBMC | T-ALL | CyTOF | 3425 | 3377 |
| 6851 | PBMC | T-ALL | CyTOF | 14617 | 1477 |
| 18067-LTB18-010 | PBMC | B-ALL | FCM | 4487 | NA |
| 18067-HTB19-289 | PBMC | B-ALL | FCM | 195 | NA |
| 18067-LTB18-578 | PBMC | B-ALL | FCM | 4324 | NA |
| 18067-HTB19-001 | PBMC | B-ALL | FCM | 2233 | NA |
| 18067-HTB19-1382 | PBMC | B-ALL | FCM | 3019 | NA |
| 18067-HTB19-048 | PBMC | B-ALL | FCM | 3412 | NA |
| 18067-HTB19-937 | PBMC | B-ALL | FCM | 4881 | NA |
| 810 | PBMC | B-ALL | FCM | 3152 | NA |
| 5385 | PBMC | T-ALL | FCM | 1715 | NA |

### Calcium Mobilization

Samples were stained with anti-CD45, anti-CD3, and anti-CD56 antibodies (Table 4). Cells were resuspended in RPMI-1640 media containing 2% FBS and stained with Indo-1/AM (1.5 μM, ThermoFisher Scientific), a UV light excitable ratiometric calcium indicator for 30 min at 37° C. Cells were stained with 7-AAD and equilibrated at 37° C. for 10 minutes for hydrolysis of AM moieties of Indo-AM dye. Calcium was measured on BD Fortessa-X20. Baseline Indo-1 fluorescence was measured for 22 seconds, followed by ionomycin treatment (1 g/mL) and measurement was continued for 3-4 minutes. Ratios of fluorescence detected at 405/20 BP (calcium-bound) to 515/20 BP (calcium-free) channels over time were calculated using derived parameters in FlowJo10.7.1 followed by normalization of fluorescence kinetics with baseline fluorescence ratio. Area under curve was calculated in GraphPad.

### CIBERSORT

CIBERSORT was carried out using https://cibersort.stanford.edu. LM22 reference was used to estimate resting and activated NK frequencies within total NK cells. To estimate NK cell frequencies with CD56^{bright} and CD56^{dim} molecular signatures by CIBERSORT, Applicant used GSE21774 to construct reference and phenotype classes files.

### Visualization of Differentially Regulated Genes

Relative signal intensities for each probeset/gene were computed by log transforming the data and centering them on the average value calculated for each gene across the immune cell types using Gene Cluster 3.0, and then visualized as heatmaps in Treeview.

### Gene Set Enrichment Analysis (GSEA)

Genes were ranked by maximum enrichment scores to identify significantly differentially regulated gene expression signatures between 100% resting and 100% activated NK groups from the hallmark gene sets provided in the molecular signatures database from Broad Institute.

### Statistics and Reproducibility

Exact p-values are provided: significant (P<0.05), trending towards significance (0.05<P<0.1). Survival was estimated by the Kaplan-Meier method. P-values were calculated using log-rank test for survival, Bonferroni method for survival analyses with multiple comparisons, GSEA method for pathway analysis, and Mann-Whitney test for all other analyses. Sample size was calculated using ‘cpower’ function in R package. Reproducibility in CyTOF was ensured by using minimum eight independent biological samples/group for the ALL cohort and minimum four samples for the healthy controls.

### Results

### NK Frequency and Cytotoxicity are Reduced in B/T-ALL Patients

To understand mechanisms underlying the suppression of NK surveillance in human ALL, using CyTOF, Applicant immunophenotyped NK cells in BMMC and PBMC of 13 B-ALL and 7 T-ALL patients (Table 5) and 22 tissue-matched healthy donors. ALL patients were selected for which therapies targeting the driver oncogene are unavailable, including those driven by rearrangements of KMT2A (5/20), CRLF2 (4/20), WYC (2/20), NOTCH1 point mutations (4/20), or CDKN2A deletions (1/20).

TABLE 5
List of high-risk B-ALL and T-ALL patient samples used in the study

| | | | | | | Translocation/ | | | |
|---|---|---|---|---|---|---|---|---|---|
| Patient | ALL | | | Tissue | | Mutation | Disease | | |
| ID | subtype | Age | Sex | Type | Cytogenetics | status | Status | Assay | Source |
| 128 | B-ALL | 9.5 | M | BMMC | 46, XY, +5, −(9)(p), −13[14]/ | CDKN2A | Diagnosis | CyTOP | Stanford |
| | | | | | 46, XY[6] | homozygous | | | |
| | | | | | | deletion (9p−/−) | | | |
| 227 | B-ALL | 4.9 | M | BMMC | Normal, 46, XY male | Normal | Diagnosis | CyTOF | Stanford |
| | | | | | karyotype | | | | |
| 268 | B-ALL | 12.1 | M | BMMC | 47 48, XY, +5, +mar[cp3]/ | Trisomy 5 | Diagnosis | CyTOF | Stanford |
| | | | | | 46, XY[18] | clone observed | | | |
| 334 | T-ALL | 17.3 | M | BMMC | 46, XY, t(10; 11) | t(10; 11) | Diagnosis | CyTOF | Stanford |
| | | | | | (p12; q14), | KMT2A | | | |
| | | | | | del(12)(p11.2)[22] | translocation | | | |
| 367 | B-ALL | 17.9 | M | BMMC | 46, XY, del(6)(q13q21), | IGH | Diagnosis | CyTOF | Stanford |
| | | | | | del(7)(p13p15)[9]/ | rearrangement | | | |
| | | | | | 46, XY[11] | (t14q32), | | | |
| | | | | | | CRLF2 | | | |
| | | | | | | overexpression, | | | |
| | | | | | | Ph-like | | | |
| 393 | B-ALL | 17.9 | M | BMMC | Normal, 46, XY male | Normal | Diagnosis | CyTOF | Stanford |
| | | | | | karyotype | | | | |
| 552 | T-ALL | 15.8 | F | BMMC | 45, XX, del(1)(p34 36), | Unknown | Diagnosis | CyTOF | Stanford |
| | | | | | del(9)(p12), add(10) | | | | |
| | | | | | (p11.2), −11, | | | | |
| | | | | | del(11)(q), −14, | | | | |
| | | | | | add(16)(p13), +mar[cp20] | | | | |
| 718 | Burkitt's | 14.1 | F | BMMC | 46, XX, del(6)(q21), | t(8; 14) - | Diagnosis | CyTOF | Stanford |
| | B cell | | | | t(8; 14)(q24; q32), | mediated MYC | | | |
| | lymphoma | | | | del(9)(q13q22) | rearrangement | | | |
| | | | | | [14]/46, XX[10] | | | | |
| 3277 | T-ALL | 12.6 | F | BMMC | 45, XX, der(8)t(8; 14)(q24; | der(8)t(8; 14) | Diagnosis | CyTOF | Stanford |
| | | | | | q11.2), −14[2]/46, XX[20] | | | | |
| 6043 | T-ALL | 37 | M | BMMC | Unknown | NOTCH1 | Diagnosis | CyTOF | UPENN |
| | | | | | | mutation | | | |
| 6457 | B-ALL | 62 | F | BMMC | Unknown | KMT2A | Diagnosis | CyTOF | UPENN |
| 6487 | T-ALL | 72 | M | BMMC | 45, X-Y[8]/46, XY[6] | NOTCH1 | Diagnosis | CyTOF | UPENN |
| | | | | | | mutation | | | |
| 18067- | B-ALL | 24 | M | BMMC | Unknown | JAK2(G); JAK2(S) | Diagnosis | CyTOF | City of |
| HTB19-222 | | | | | | | | | Hope |
| 65 | B-ALL | 33 | F | PBMC | 47-48, xx, −4-11, +3-4 | t(4; 11)KMT2A | Diagnosis | CyTOF | UPENN |
| | | | | | probable t(4; 11) | translocation | | | |
| 779 | B-ALL | 48 | F | PBMC | 46, XX, t(1; 11)(p32; q23) | t(1; 11)KMT2A | Diagnosis | CyTOF | UPENN |
| | | | | | [10]/48, idem, +X, +21[10]/ | translocation | | | |
| | | | | | fish for KMT2A | | | | |
| | | | | | split pos 163/200 cells/ | | | | |
| | | | | | fish for bcr-abl neg | | | | |
| 810 | B-ALL | 62 | M | PBMC | 46, XY[25] | Unknown | Diagnosis | CyTOF/Flow | UPENN |
| | | | | | | | | cytometry | |
| 2142 | B-ALL | 30 | M | PBMC | 46, XY, del(9)(p21p21)[6]/ | Ph-like | Diagnosis | CyTOF | UPENN |
| | | | | | 46, XY[24] | | | | |
| 3113 | B-ALL | 44 | F | PBMC | Unknown | KMT2A/AFF1 | Diagnosis | CyTOF | UPENN |
| 4986 | B-ALL | 41 | M | PBMC | 46, XY[5] | Ph-like | Diagnosis | CyTOF | UPENN |
| 4988 | B-ALL | 61 | F | PBMC | 46, XX, del(7)(p11.2)[7]/ | Ph-like | Refractory | CyTOF | UPENN |
| | | | | | 46, XX[13] | | | | |
| 5921 | T-ALL | 32 | M | PBMC | 46, XY[20] | NOTCH1 | Diagnosis | CyTOF | UPENN |
| | | | | | | mutation | | | |
| 6070 | T-ALL | 46 | M | PBMC | Unknown | NOTCH1 | Diagnosis | CyTOF | UPENN |
| | | | | | | mutation | | | |
| 6851 | T-ALL | 50 | F | PBMC | 46, XX[7].ish | Unknown | Diagnosis | CyTOP | UPENN |
| | | | | | (ABL1amp, BCRx2) [1] | | | | |
| 18067- | B-ALL | 24 | M | PBMC | Unknown | JAK2(G); JAK2(S) | Diagnosis | CyTOF | UPENN |
| LTB18-544 | | | | | | | | | |
| 5385 | T-ALL | 79 | F | PBMC | 46, XX, ins(22; ?)(p11.2; | NOTCH1 | Diagnosis | Flow | UPENN |
| | | | | | ?) [14]/46, XX[6] | mutation | | Cytometry | |
| 18067- | B-ALL | 57 | F | PBMC | Unknown | JAK2(G); JAK2(S) | Diagnosis | NK | City of |
| HTB18-029 | | | | | | (Ph-like) | | Cytotoxicity | Hope |
| 18067- | B-ALL | 24 | M | PBMC | Normal | EZH2; ETV6; | Diagnosis | Flow | City of |
| HTB19-1382 | | | | | | KMT2D | | Cytometry | Hope |
| 18067- | B-ALL | 44 | F | PBMC | Normal | KMT2D | Diagnosis | Flow | City of |
| LTB18-578 | | | | | | | | Cytometry | Hope |
| 18067- | B-ALL | 20 | F | PBMC | 47, XX, +22[6] | JAK2; JAK1 | Diagnosis | Nk | City of |
| HTB19-048 | | | | | | (Ph-like) | | cytotoxicity, | Hope |
| | | | | | | | | Calcium | |
| | | | | | | | | Flux, Flow | |
| | | | | | | | | cytometry | |
| 18067- | B-ALL | 41 | F | PBMC | 46, XX[16].ish t(X; 14) | IKZF1; JAK2(G); | Diagnosis | Calcium | City of |
| HTB19-937 | | | | | (p22.33; q32.33) | JAK2(S); PAX5 | | Flux, Flow | Hope |
| | | | | | (5′IGH+; 3′IGH+)[2] | (Ph-like) | | cytometry | |
| 18067- | B-ALL | 35 | F | PBMC | 47, XX, +X[9] | JAK2; NRAS | Diagnosis | Calcium | City of |
| HTB19-001 | | | | | | (Ph-like) | | Flux, Flow | Hope |
| | | | | | | | | cytometry | |
| 18067- | B-ALL | 30 | F | PBMC | Unknown | KMT2D | Diagnosis | Calcium | City of |
| HTB19-376 | | | | | | | | Flux | Hope |
| 18067- | B-ALL | 20 | F | PBMC | 50, XX, +X, +2, +4, t(9; | Hi Risk, no | Diagnosis | Flow | City of |
| LTB18-010 | | | | | 22) (q34.1; q11.2), +der | Mutations | | cytometry | Hope |
| | | | | | (22)t(9; 22)[4] | | | | |
| 18067- | B-ALL | 43 | F | PBMC | 47, XX, −2, t(3; 15)(p23; | KRAS; KMT2D; | Diagnosis | Flow | City of |
| HTB19-289 | | | | | q15), del(5)(q22q3?3), | PAX5 | | cytometry | Hope |
| | | | | | del(7)(p13p15), +del(9) | | | | |
| | | | | | (p21.2), der(9)del(9) | | | | |
| | | | | | (p13p22)del(9)(q22)x2, | | | | |
| | | | | | der(10)t(2; 10)(q21; q26), | | | | |
| | | | | | del(12)(p11.2p13.3), | | | | |
| | | | | | add(17) | | | | |
| | | | | | (q25)x2, −20, +21, +mar | | | | |
| | | | | | [17] | | | | |
| 18067- | B-ALL | 24 | M | PBMC | 47, XY, +X[6] | JAK2(G); JAK2(S) | Diagnosis | Calcium | City of |
| LTB18-544 | | | | | | (Ph-like) | | Flux | Hope |
| 18067- | B-ALL | 54 | F | PBMC | 46, XX, t(9; 22)(q34.1; | KMT2C | Diagnosis | NK | City of |
| HTB19-1420 | | | | | q11.2)[6]; 48, | | | cytotoxicity | Hope |
| | | | | | sl, +4, −16, +21, | | | | |
| | | | | | der(22)t(9; 22)add | | | | |
| | | | | | (9)(q34.3), +der(22)t(9; | | | | |
| | | | | | 22)add (9)[11]47, sdl1, | | | | |
| | | | | | t(5; 12) (q33; q13), −21[3] | | | | |

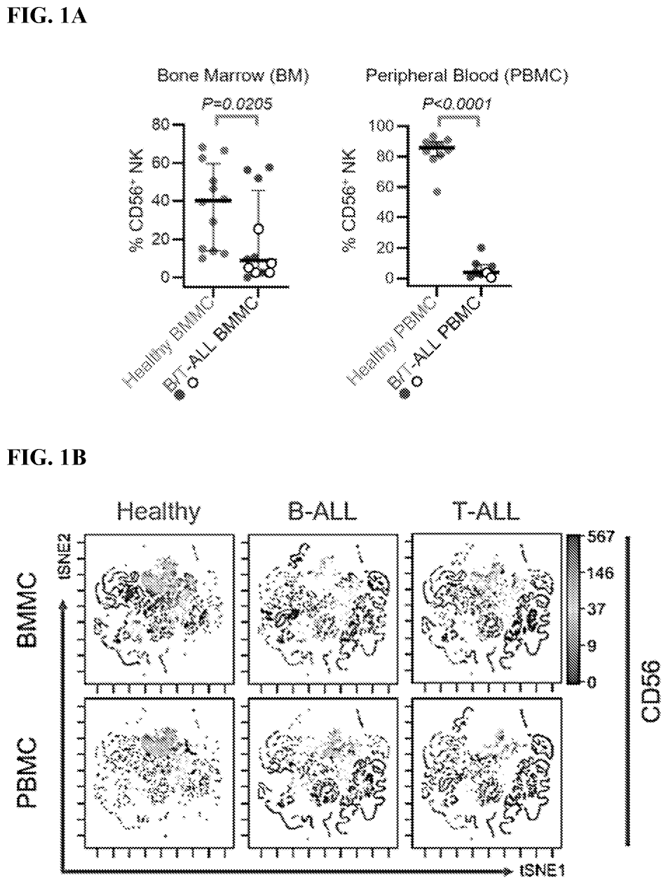

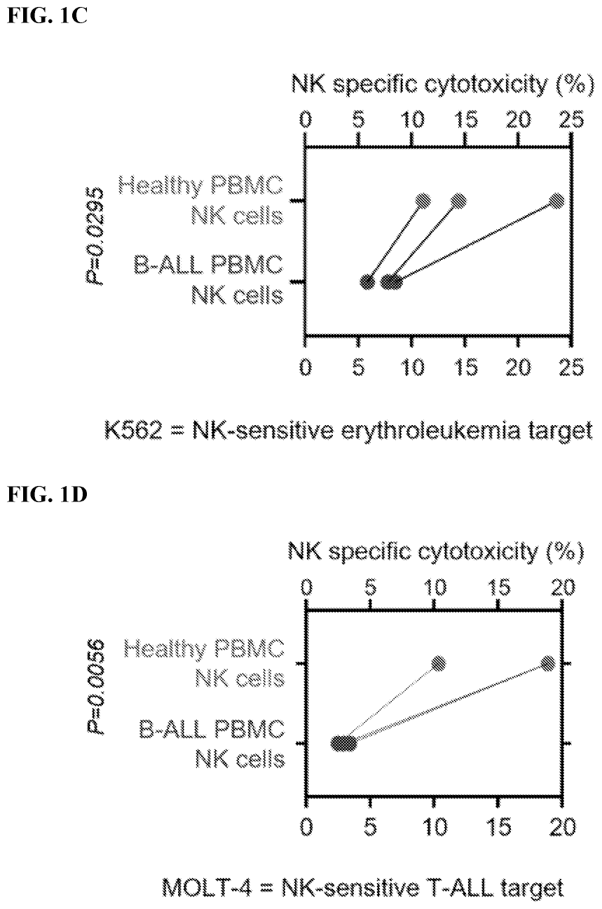

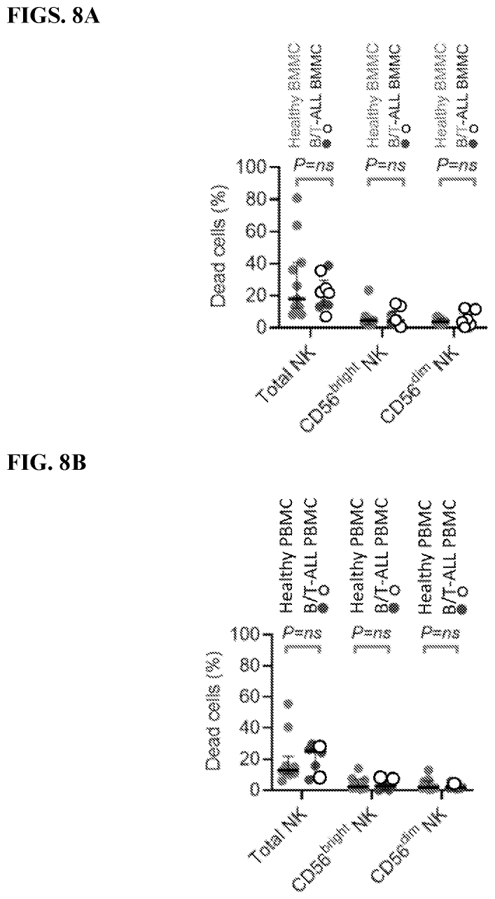

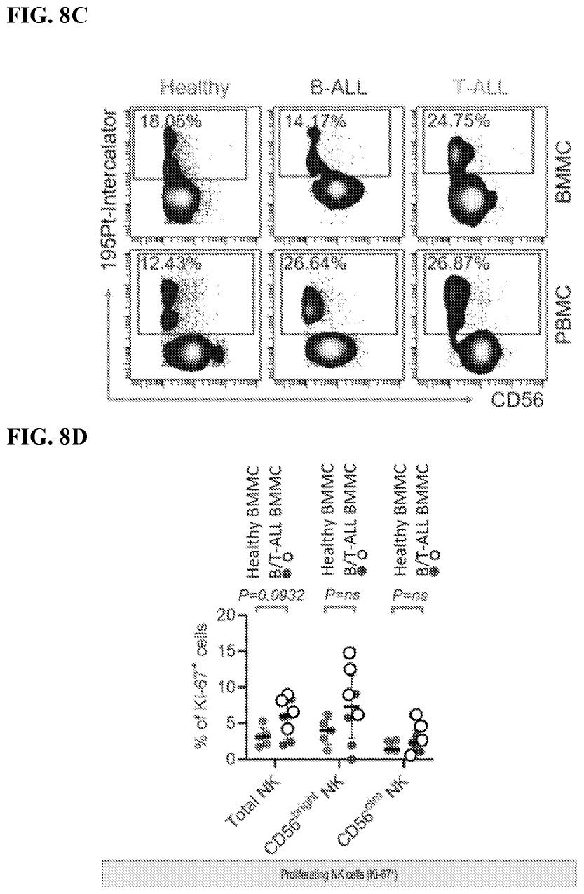

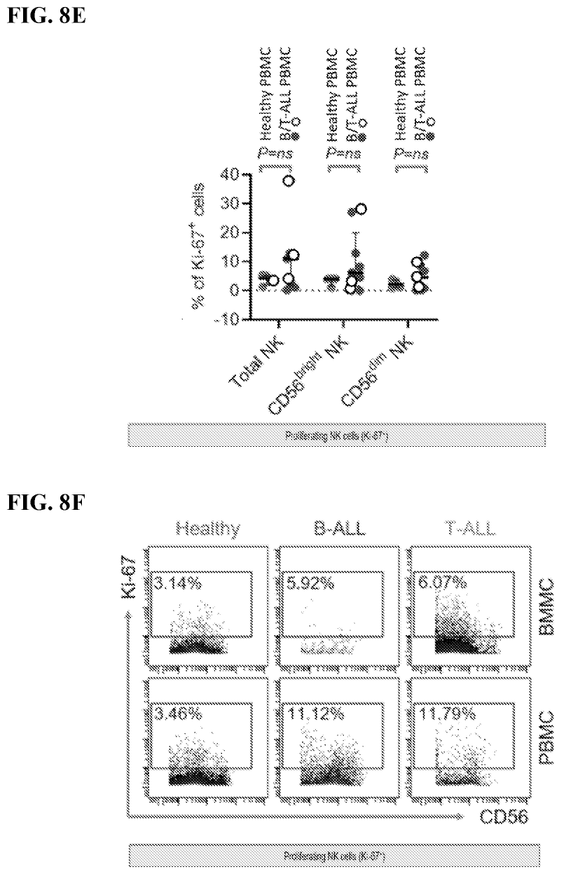

Frequencies of CD56⁺ NK cells were first compared between B/T-ALL patients and healthy donors after gating out CD14⁺ and/or CD33⁺ myeloid cells, CD3⁺ T/NKT cells, and CD19⁺ and/or CD20⁺ B cells. Consistent with reduced NK numbers previously reported in murine ALL, NK frequencies were significantly reduced within the non-malignant immune fraction of ALL patients compared to healthy donors (FIGS. 1A-1B). Because no differences were observed in NK viability and proliferation between patients and healthy donors (FIGS. 8A-8F), it was concluded that NK frequencies in B/T-ALL are likely reduced by other mechanisms.

Applicant then examined whether cytotoxicity of residual NK cells is impaired in ALL patients by comparing the abilities of sorted PBMC NK cells from B-ALL patients and healthy donors to lyse allogeneic NK-sensitive erythroleukemia and T-ALL targets in vitro. ALL NK cells exhibited significantly lower specific cytotoxicity towards leukemia targets compared to healthy donor NK cells. Therefore, reduced NK cells and suppressed NK cytotoxicity block NK surveillance in ALL patients.

NK Cells with Less Cytotoxic CD56^{bright} Molecular Signature are Expanded in B/T-ALL

Reduced cytotoxicity of ALL NK cells suggests that production of cytolytic NK effectors is perturbed in ALL patients. Human NK cells mature from less cytotoxic CD56^{bright}CD16⁻ to highly cytotoxic CD56^{dim} CD16⁺ stages. It was postulated that frequencies of CD56^{bright} and CD56^{dim} NK cells are perturbed in ALL.

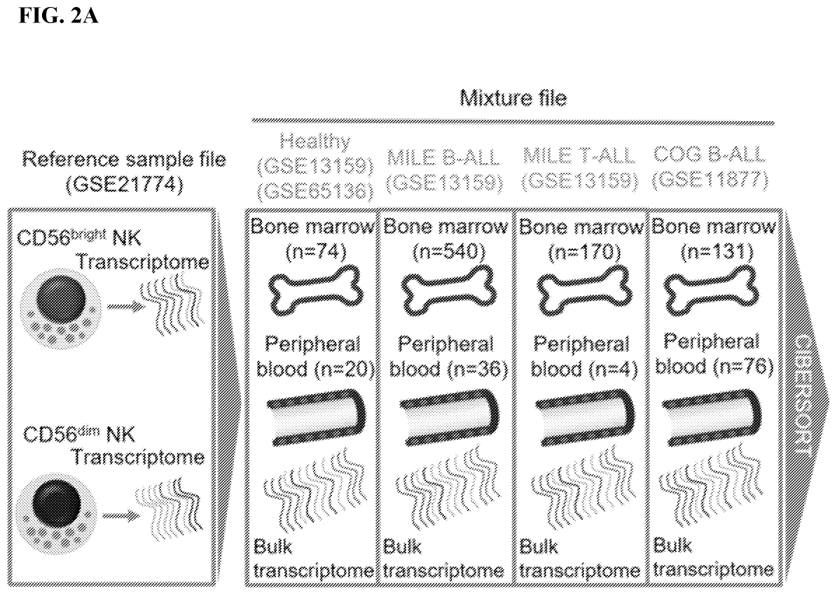

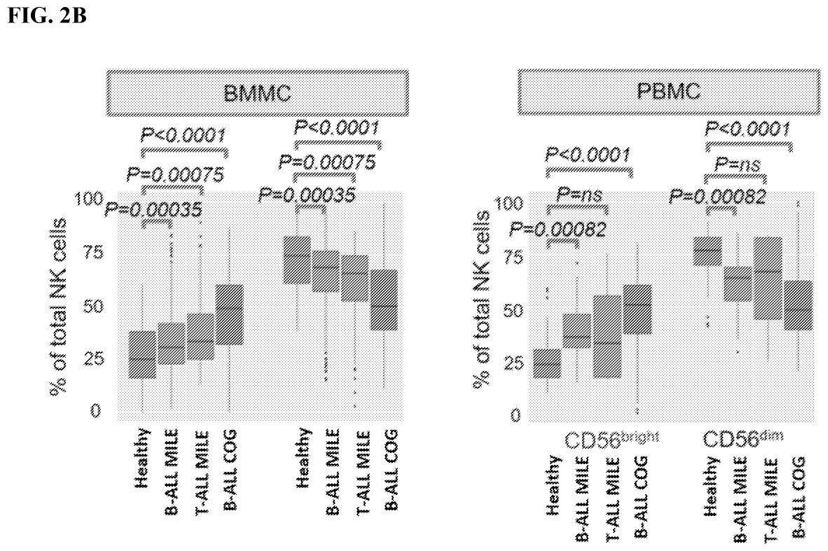

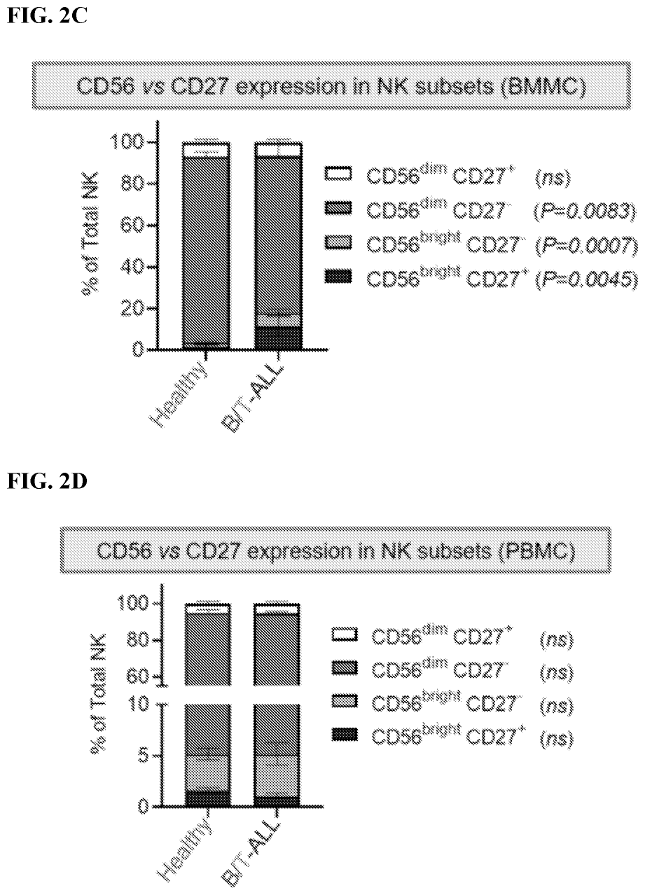

Using CIBERSORT, relative frequencies were compared of NK cells with CD56^{bright} and CD56^{dim} molecular signatures (GSE21774) in 94 healthy donors (GSE65136, GSE13159) against 207 B-ALL from P9906 Children's Oncology Group (COG) clinical trial (GSE11877), and 576 B-ALL and 174 T-ALL from Microarray Innovations in Leukemia (MILE) (GSE13159) banking study (FIG. 2A). Transcriptomes of patient samples in COG and MILE were measured at diagnosis (pre-treatment). Concordant with reduced NK cytotoxicity in ALL, it was found that frequencies of NK cells with CD56^{bright} molecular signature were increased while those with CD56^{dim} signature were reduced in B/T-ALL (FIG. 2B).

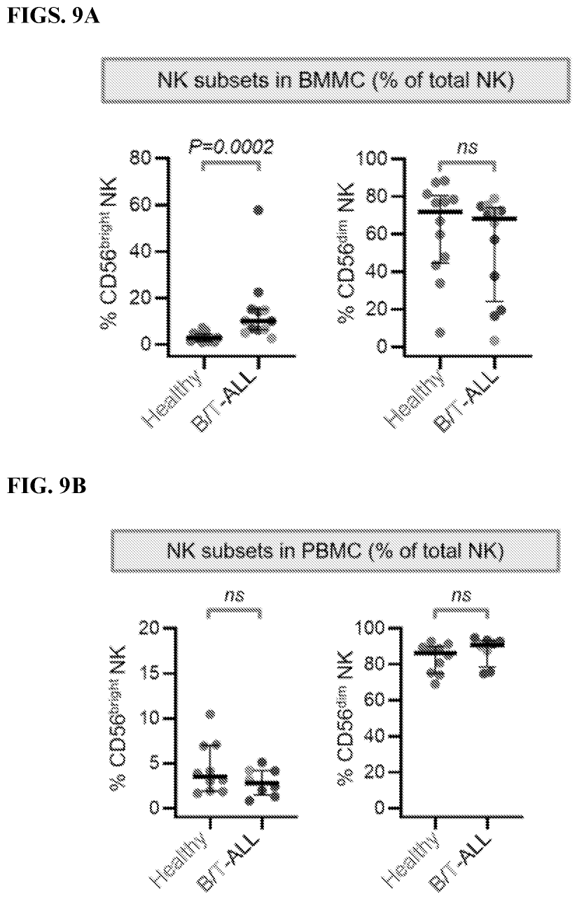

Next, using CyTOF, Applicant compared CD56^{bright} and CD56^{dim} NK frequencies in B/T-ALL and healthy donors. Consistent with CIBERSORT, a significant increase was observed in CD56^{bright} NK fraction in BMMC of B/T-ALL patients (FIG. 9A). Surprisingly, CD56^{bright} and CD56^{dim} NK frequencies were unchanged in PBMC of B/T-ALL patients compared to healthy donors (FIG. 9B), albeit CIBERSORT showed that cells with CD56^{bright} NK transcriptome were enriched in ALL (FIG. 2B). While CIBERSORT considers a comprehensive molecular signature of the CD56^{bright} and CD56^{dim} NK fractions, CyTOF discriminates CD56^{bright} and CD56^{dim} subsets based on CD56 expression, which, without being bound to any theory, could explain the discrepancy between the two methods. It was inferred that ALL NK cells, irrespective of their surface CD56 expression, have the molecular makeup of less mature and less cytotoxic CD56^{bright} NK cells. Proliferation and viability of CD56^{bright} and CD56^{dim} NK subsets are unaffected in ALL (FIGS. 8A-8F). Hence, their perturbed turnover does not lead to the expansion of NK cells with the CD56^{bright} molecular phenotype in ALL patients.

Next, co-expression of CD27 with CD56 was examined. Majority of CD56^{bright} NK cells are CD27⁺ and differentiation into CD56^{dim} cytotoxic effectors results in CD27 loss. A significant increase in the least cytotoxic CD56^{bright}CD27⁺NK fraction and a concomitant decrease in the cytotoxic CD56^{dim}CD27− NK subset in BMMC but not PBMC of patients was observed (FIGS. 2C-2D).

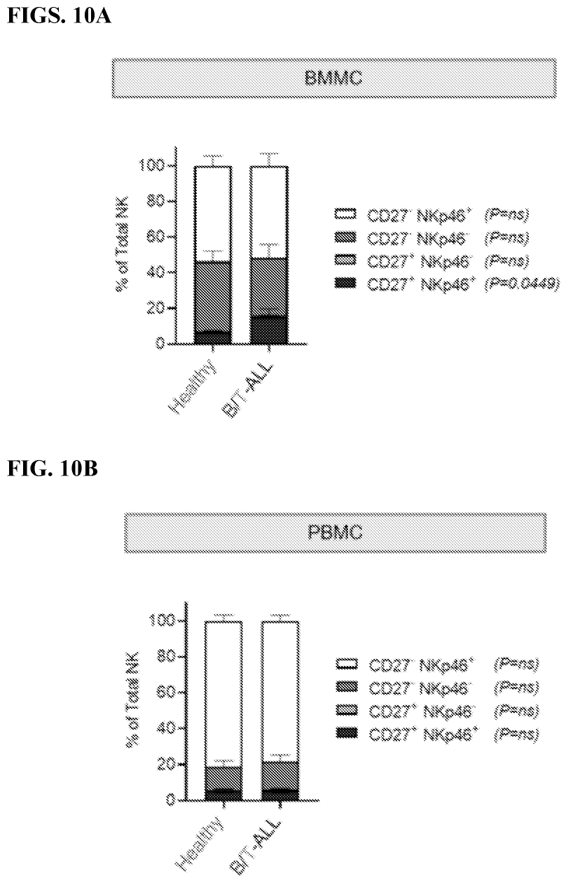

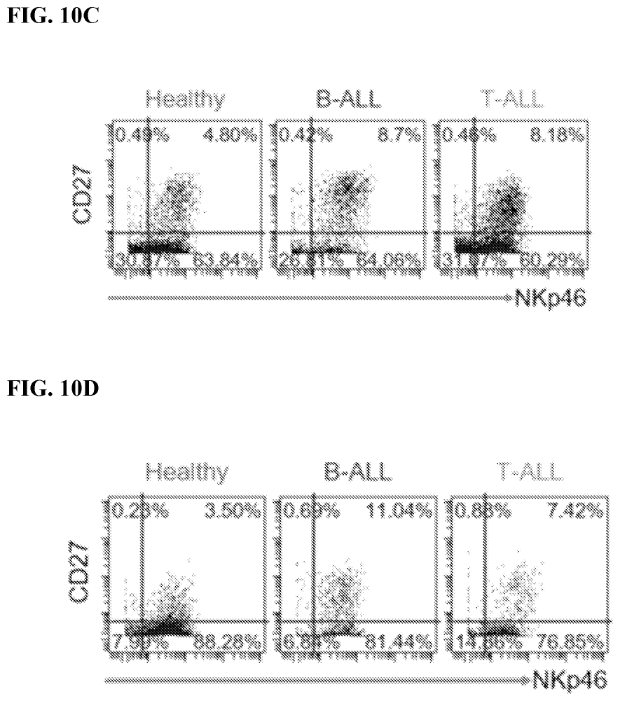

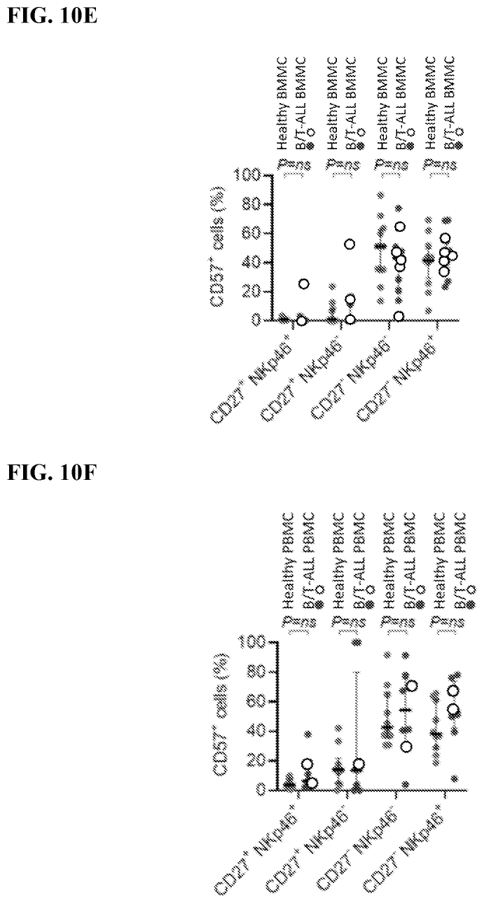

Finally, expression of the natural cytotoxicity receptor NKp46 were compared, which reduces during NK transition from CD56^{bright} to CD56^{dim} stages between patients and healthy donors. Less cytolytic CD56^{bright}CD27⁺NK cells express more NKp46 than their CD56^{dim} CD27− counterparts. Significantly increased frequencies of CD27⁺NKp46⁺ NK cells in BMMC of ALL patients compared to healthy donors were observed, while no significant changes were observed in the PBMC (FIGS. 10A-10D). Low levels of the NK maturation marker CD57 confirmed that CD27⁺NKp46⁺ NK cells were immature (FIGS. 10E-10F).

Increase in immature CD56^{bright}CD27⁺ and CD27⁺NKp46⁺ NK cells in BMMC corroborated the expansion of less cytotoxic CD56^{bright} NK cells in BMMC of ALL patients (FIGS. 2C, 9A-9B, 10A, 10C). For PBMC, despite no perturbations in CD56, CD27, and NKp46 in ALL NK cells (FIGS. 2D, 9B, 10B, 10D), it was observed that NK cytotoxicity was reduced and NK cells with CD56^{bright} transcriptome were enriched (FIG. 2B). Therefore, it was inferred that NK cells in PBMC of ALL patients exhibit CD56^{bright}-like characteristics.

Maturation of NK Cells into Cytotoxic Effectors is Perturbed in PBMC of B/T-ALL Patients

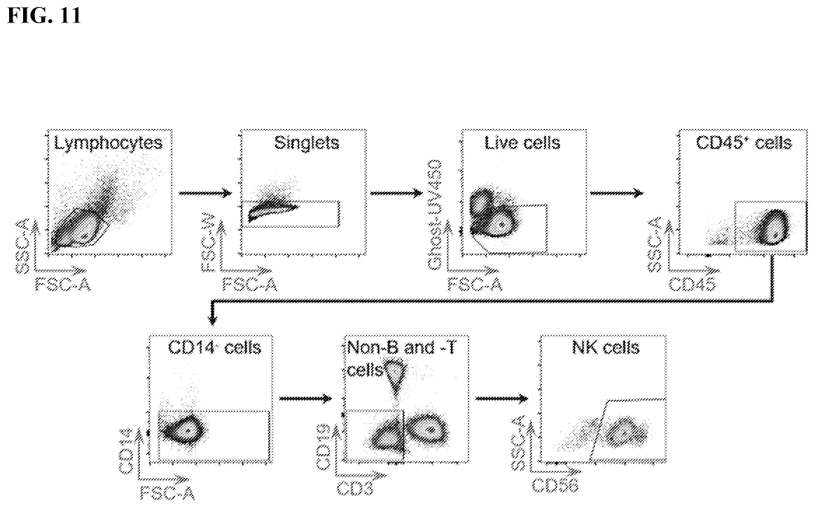

To understand why NK cells in PBMC of ALL patients are less cytotoxic despite the absence of perturbations in CD56 and CD27, the CD56^{bright} to CD56^{dim} NK transition was characterized by comparing CD94, KIR2DL1, NKG2A, and CD62L on NK cells in PBMC of 9 healthy donors and 1 T- and 8 B-ALL patients by flow cytometry (FIG. 11).

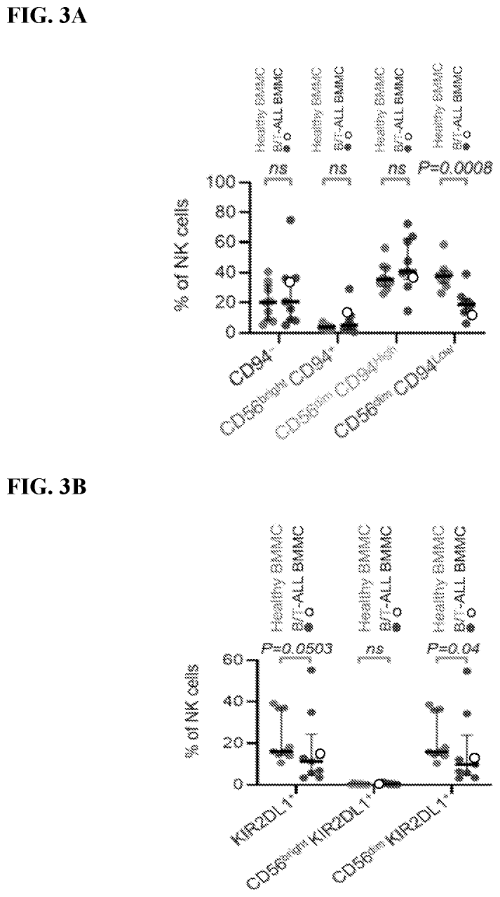

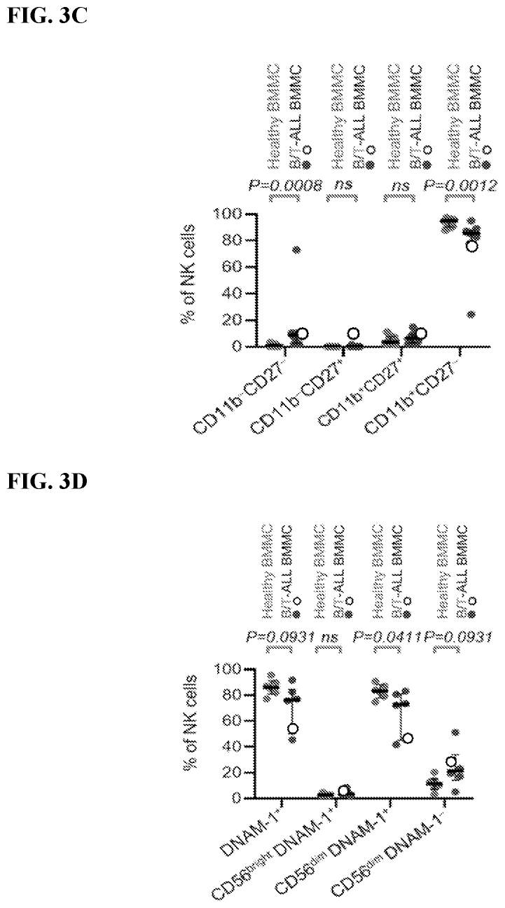

Surface density of CD94 is reduced in a stepwise fashion as NK cells differentiate from CD56^{bright} to CD56^{dim} stages: less cytotoxic CD56^{bright}CD94^{High} cells transition into a more cytotoxic CD56^{dim}CD94^{High} intermediary before finally maturing into the most cytotoxic CD56^{dim} CD94^{Low} subset. Significantly decreased frequencies of the most cytotoxic CD56^{dim} CD94^{Low} subset in B/T-ALL patients was observed (FIG. 3A). The inhibitory receptor KIR2DL1, expressed more by CD56^{dim} than CD56^{bright} NK cells, was also reduced in total and CD56^{dim} ALL NK cells (FIG. 3B). No changes were observed for NKG2A and CD62L between ALL and healthy PBMC (not shown).

The data suggested that the pathway producing cytotoxic NK effectors is perturbed in ALL. NK effector maturation is a 4-stage process with progressive acquisition of cytotoxicity: CD11b⁻CD27− double negative (DN)→CD11b⁻CD27⁺ single positive (SP)→CD11b⁺CD27⁺ double positive (DP)→CD11b⁺CD27− (SP). Significantly increased frequencies of immature and least cytotoxic CD11b⁻CD27− DN NK cells and reduced frequencies of cytotoxic CD11b⁺CD27− SP NK effectors was observed (FIG. 3C). The CD11b⁺CD27− fraction resembles the CD56^{dim} NK subset while the other fractions resemble CD56^{bright} NK cells, further supporting our observations that NK cells with the CD56^{bright} molecular phenotype are enriched in ALL patients.

Finally, the expression of the cytotoxicity-inducing activating receptor DNAM-1 in ALL patients and healthy donors was observed. A trend toward reduced DNAM-1⁺ total NK, significantly reduced frequencies of the most cytotoxic CD56^{dim} DNAM1⁺NK was observed, and a trend toward increased CD56^{dim} DNAM-1⁻ less cytotoxic fraction in patients compared to healthy donors (FIG. 3D). Concordant with studies showing that the CD56^{dim} DNAM-1⁺ NK cell subset has maximal effector function, the findings demonstrate defective NK effector maturation in ALL patients.

It was concluded that perturbations in NK effector maturation from the CD56^{bright} to CD56^{dim} NK subset result in accumulation of dysfunctional NK cells in B/T-ALL patients.

Stimulated ALL NK Cells Produce More Cytokines than their Healthy Counterparts

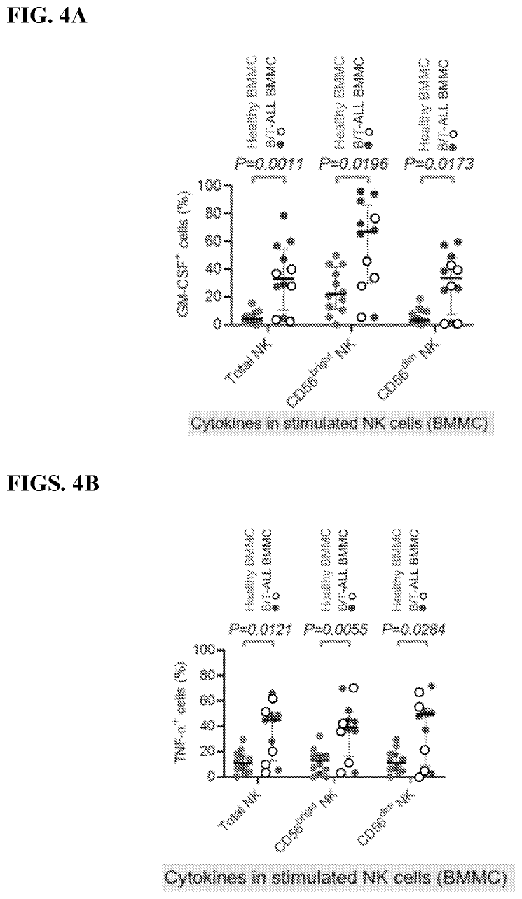

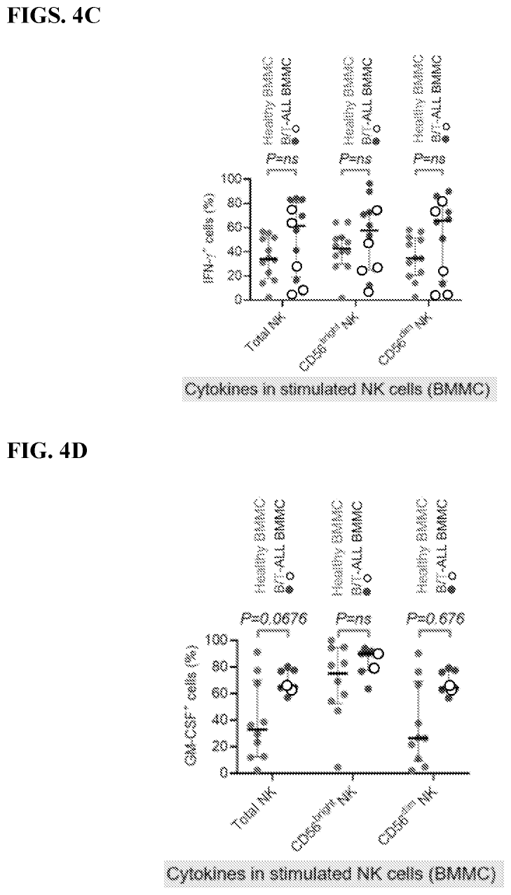

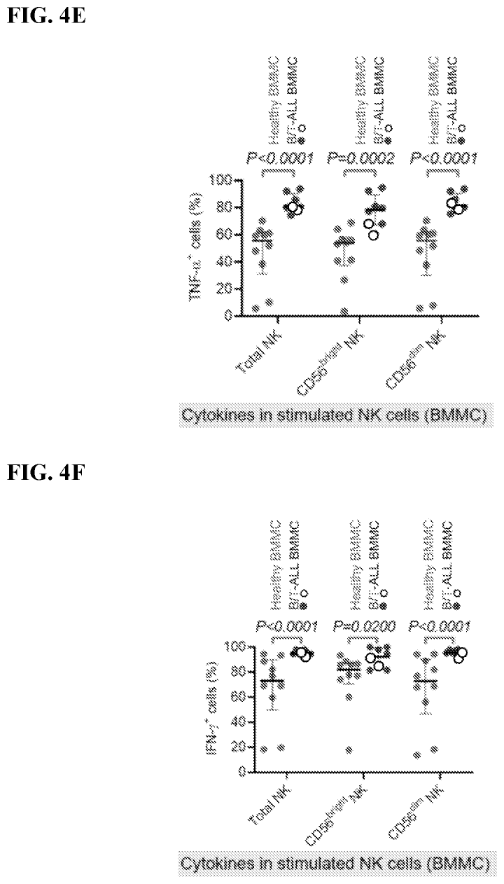

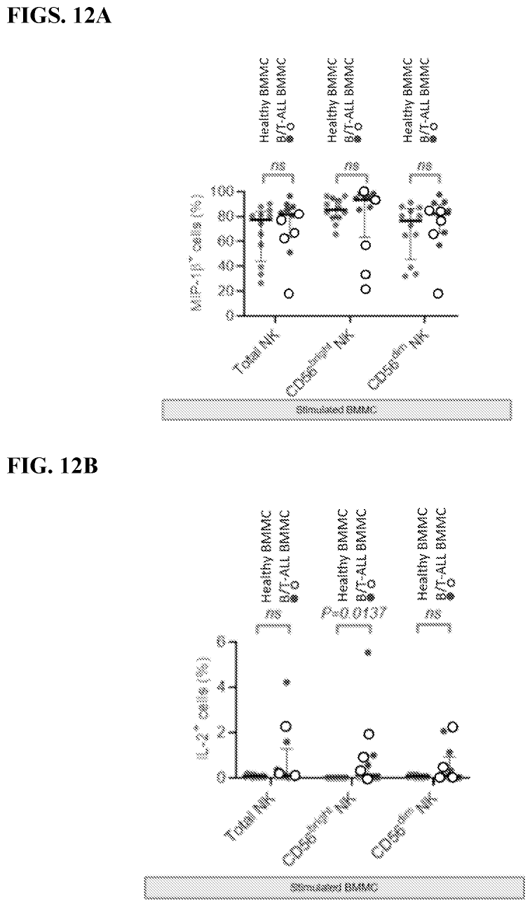

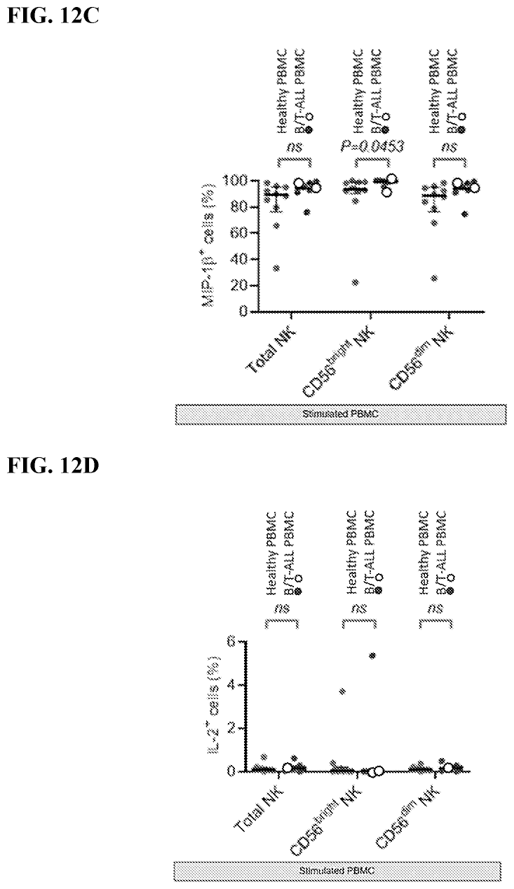

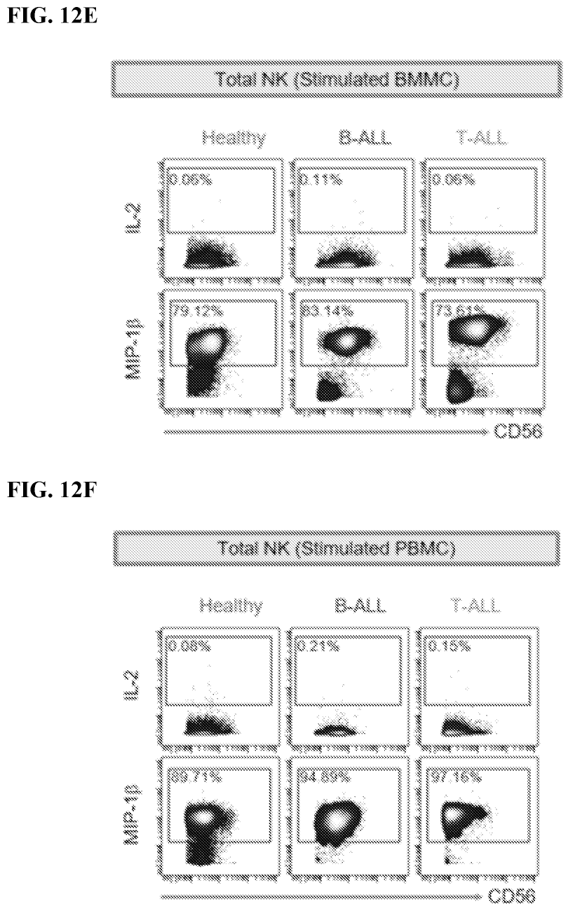

Upon stimulation with PMA+ionomycin or monokines, CD56^{bright} NK cells express more cytokines including IFN-γ, TNF, and GM-CSF than their CD56^{dim} counterparts. Because NK cells with CD56^{bright} signature are increased in B/T-ALL (FIGS. 2A-2D, 3A-3D, 9A-9B, 10A-10F), Applicant predicted that stimulated ALL NK cells will produce more cytokines than their normal counterparts. To test this, frequencies of PMA+ionomycin-stimulated NK subsets expressing intracellular cytokines in B/T-ALL patients and healthy controls was compared. It was found that increases in frequencies of GM-CSF⁺ and TNF-α+NK cells in BMMC and PBMC of B/T-ALL patients compared to healthy donors. IFN-γ⁺ NK cells were significantly increased in PBMC but unchanged in BMMC of B/T-ALL patients compared to healthy individuals (FIGS. 4A-4F). It was also found that significant increases in MIP-1β⁺ and IL-2⁺ cells within CD56^{bright} NK fraction of PBMC and BMMC respectively, in ALL patients (FIG. 12). Hence, stimulated ALL NK cells produce more cytokines than their healthy counterparts, which further corroborates the expansion of immature, cytokine-producing, and poorly cytotoxic CD56^{bright}-like NK cells in ALL patients.

Peripheral Blood ALL NK Cells Degranulate More than Healthy NK Cells

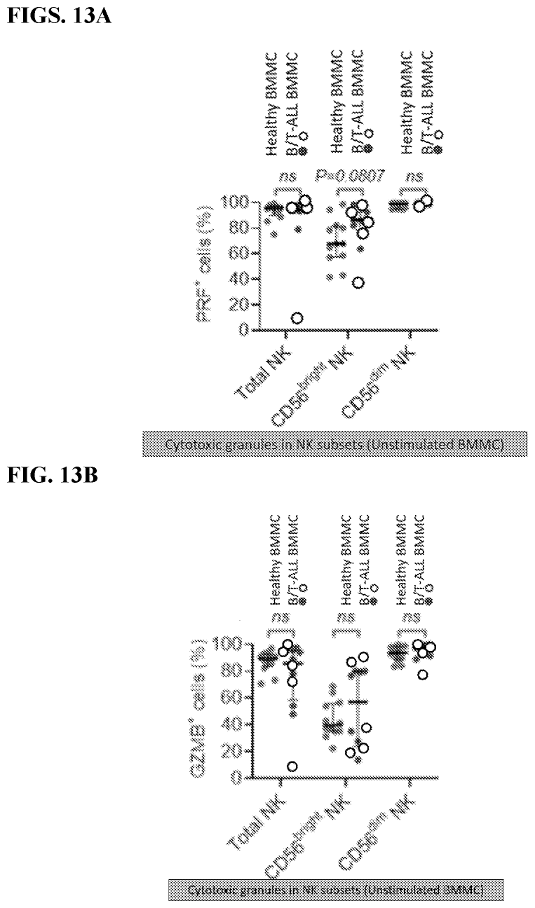

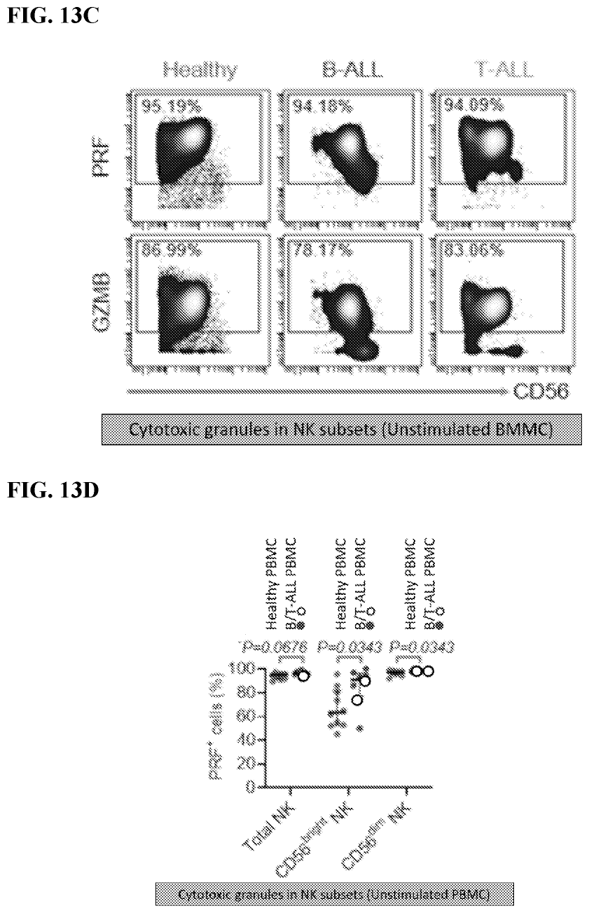

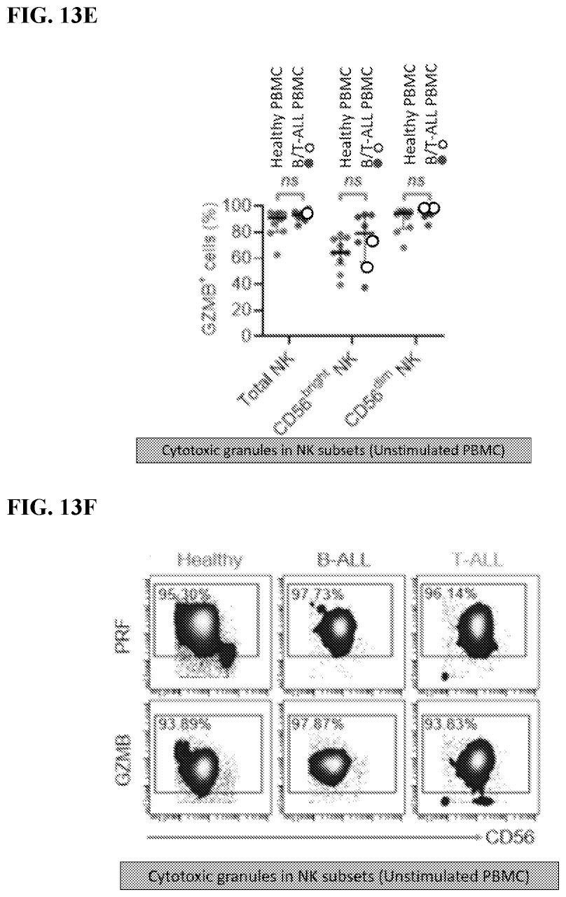

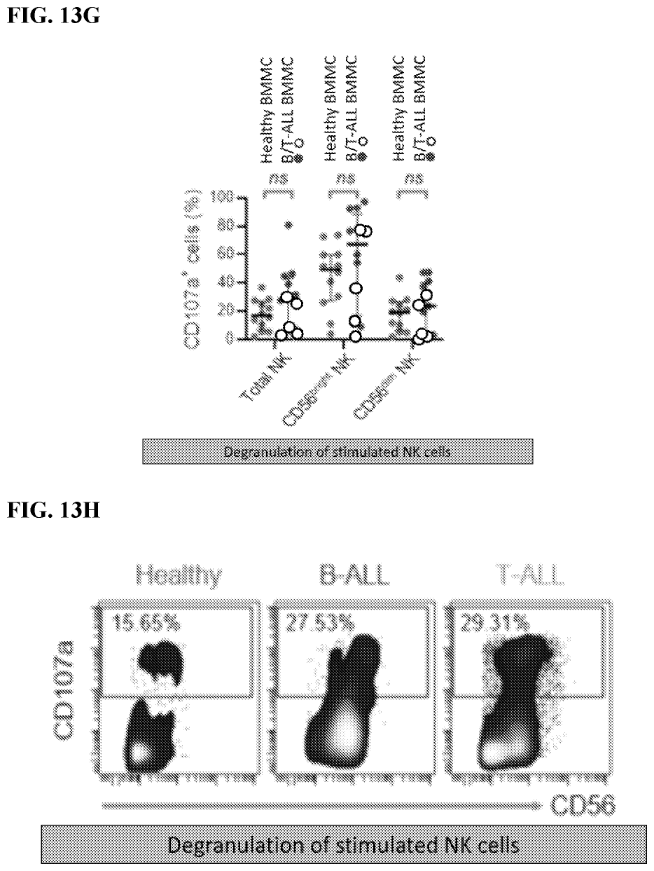

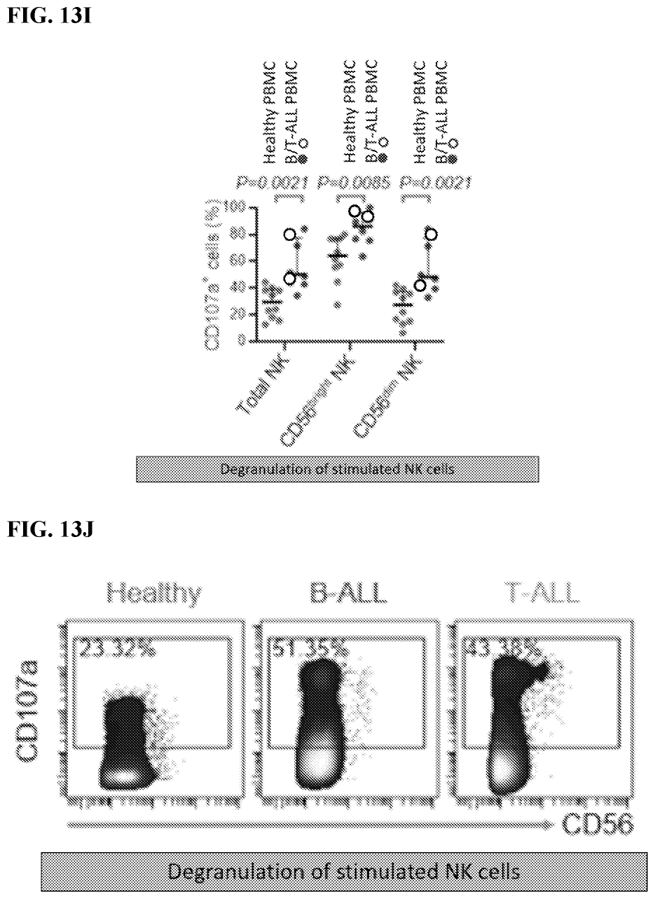

Because CD56^{bright} NK cells express less cytotoxic granules than their CD56^{dim} counterparts, expression of cytotoxic granules (PRF, GZMB) between ALL and healthy NK cells was compared. Surprisingly, a trend of increased PRF⁺CD56^{bright} NK in BMMC and PRF⁺ total NK in PBMCs was observed, and significant increases in frequencies of PRF⁺ NK subsets in ALL PBMC (FIGS. 13A-13F).

Stimulated CD56^{bright} NK cells degranulate more and express more CD107a than their CD56^{dim} counterparts. The frequencies of CD107a⁺ stimulated NK cells between ALL patients and healthy donors was observed. Although Applicant observed no significant changes in frequencies of CD107a⁺ BMMC NK cells, a striking increase in percentages of CD107a⁺ cells in stimulated PBMC NK fractions of ALL patients compared to healthy donors was measured (FIGS. 13G-13J).

### Hyperactivated and Exhausted NK Cells Accumulate in Peripheral Blood of ALL Patients

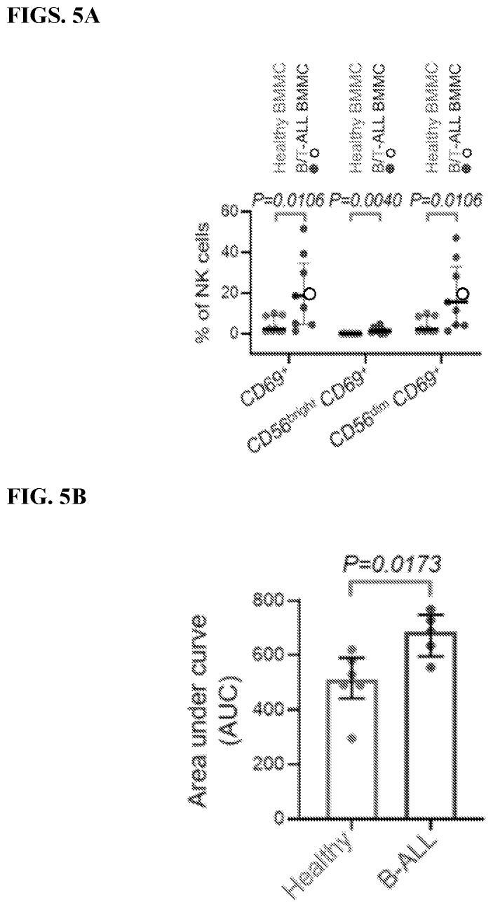

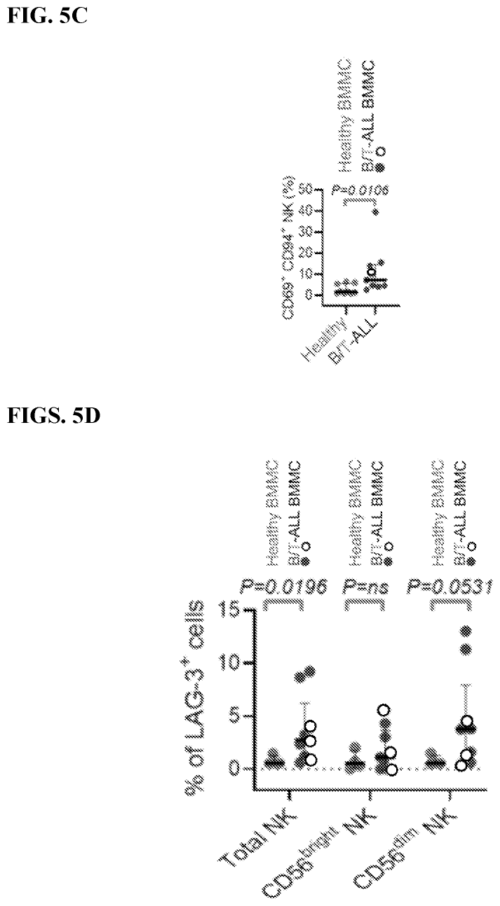

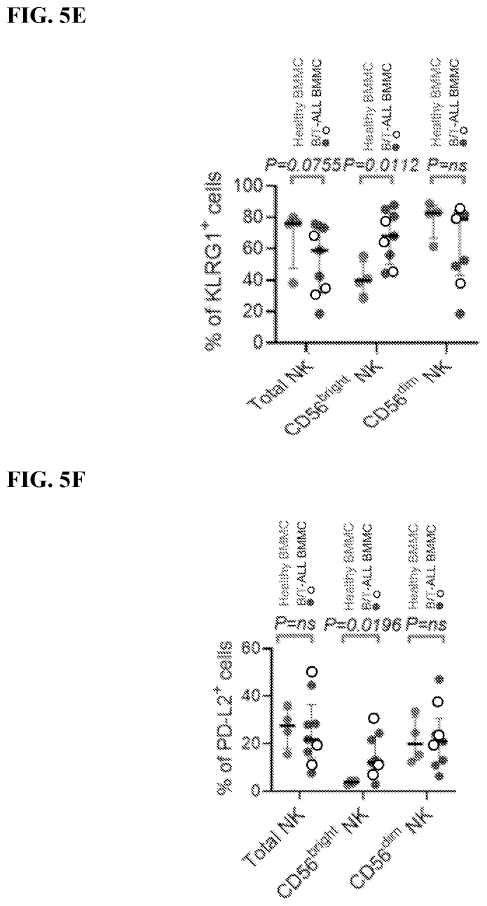

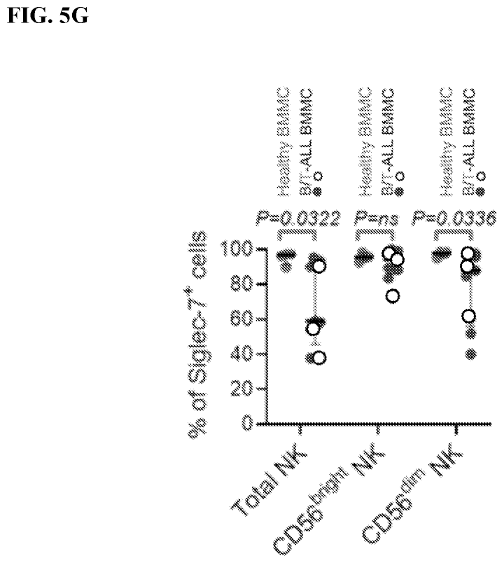

Increased expression of cytokines, lytic granules and CD107a upon stimulation (FIGS. 4A-4F, 13A-13J) suggest that PBMC NK cells in ALL patients are hyperactivated. This was tested by comparing the levels of the activation marker CD69 and calcium (Ca²⁺) mobilization in NK cells of ALL patients and healthy donors by flow cytometry. A significant increase in CD69⁺ cells in total NK and NK subsets of ALL patients was observed (FIGS. 5A-5F) and found that ALL NK cells exhibit higher calcium flux (FIG. 5B). ALL NK cells are therefore more activated than their healthy counterparts.

Despite their increased activation, PBMC ALL NK cells cannot lyse NK-sensitive leukemia targets. Applicant investigated two mechanisms that could explain this dysfunctionality of hyperactivated ALL NK cells.

First, the expression of CD94 on CD69⁺NK cells in ALL patients and healthy donors was compared because CD94 inhibits CD69-mediated NK cytotoxicity, and CD94 is perturbed in ALL NK cells (FIG. 3A). A significant increase in CD69⁺CD94⁺NK cells in ALL patients was observed (FIG. 5C). These observations, together with others' studies showing that excessive Ca²⁺ signaling impedes NK-mediated lysis of cancers, explain at least partly why NK cells in ALL, despite being highly activated, are dysfunctional.

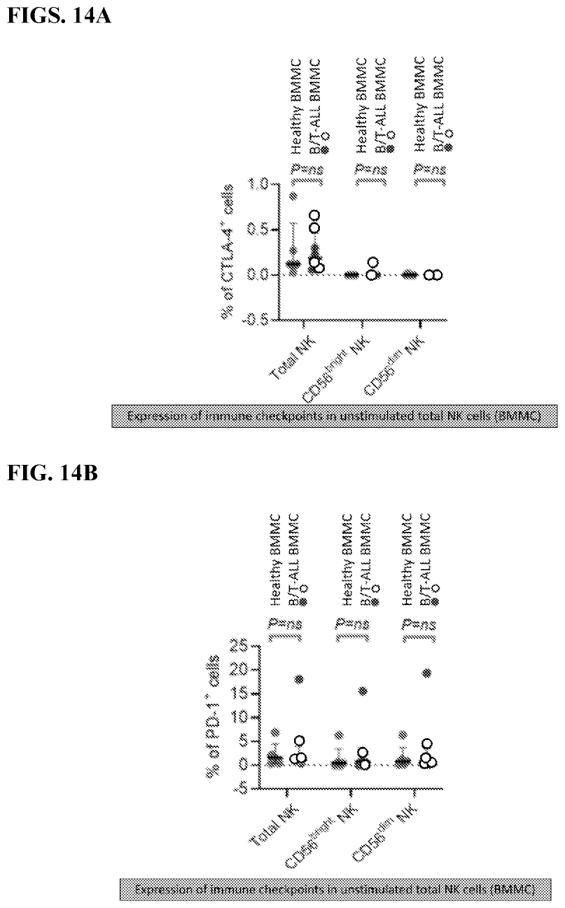

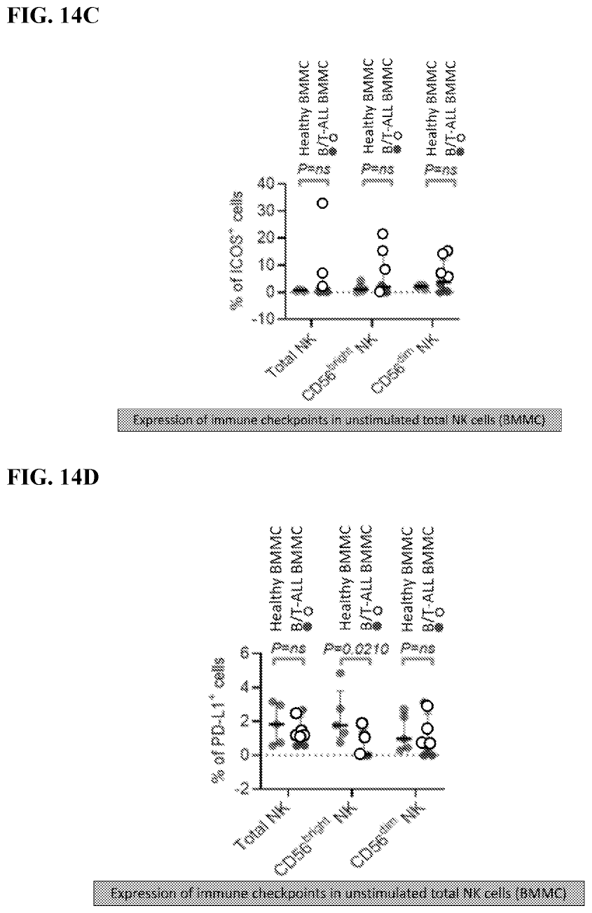

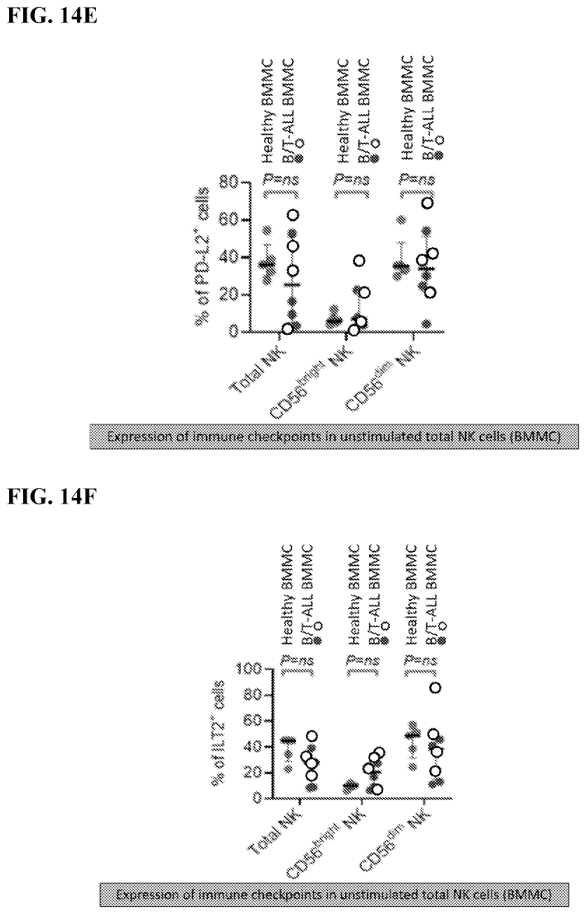

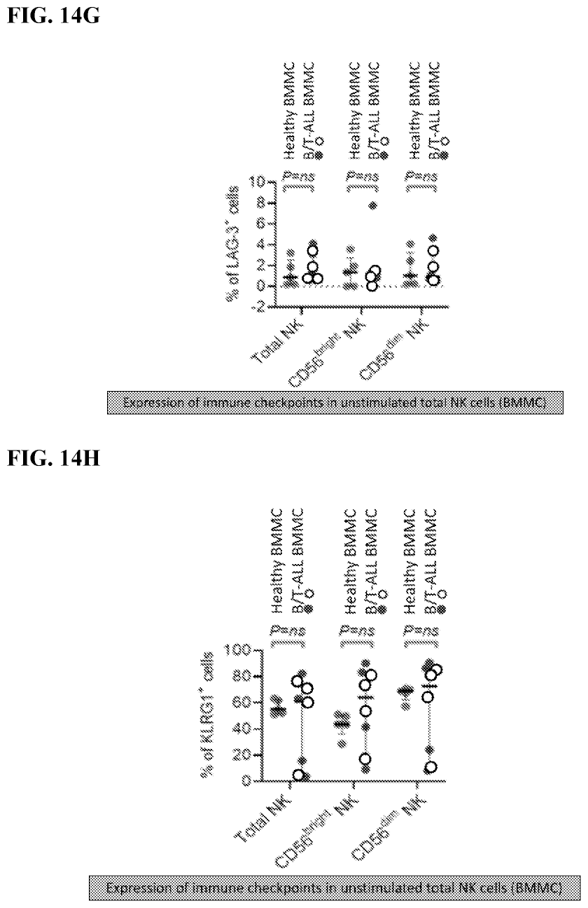

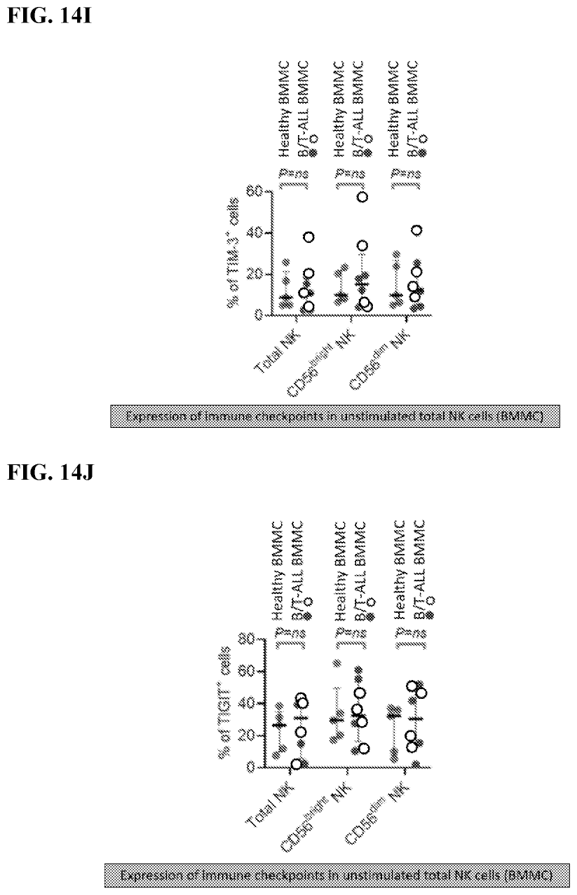

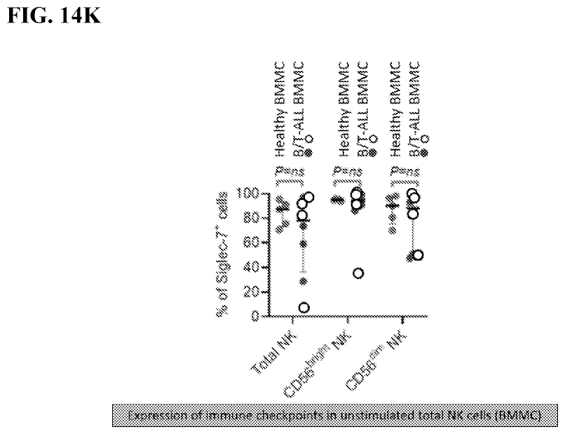

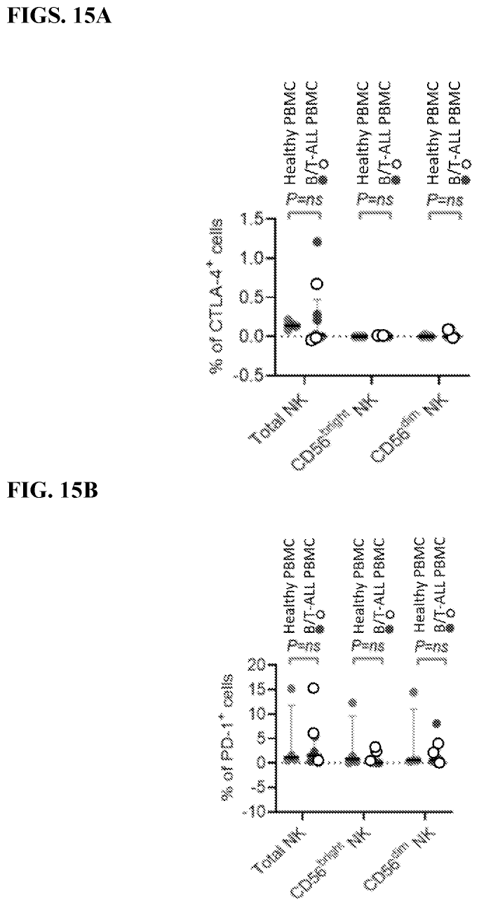

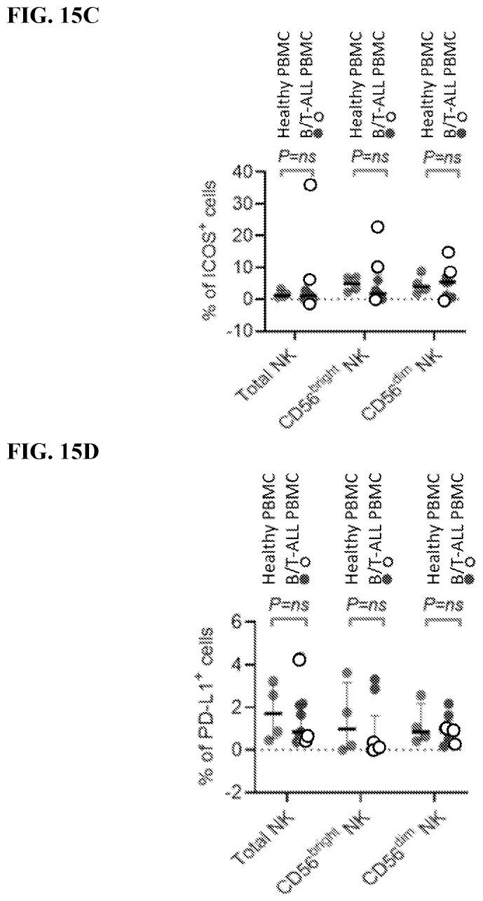

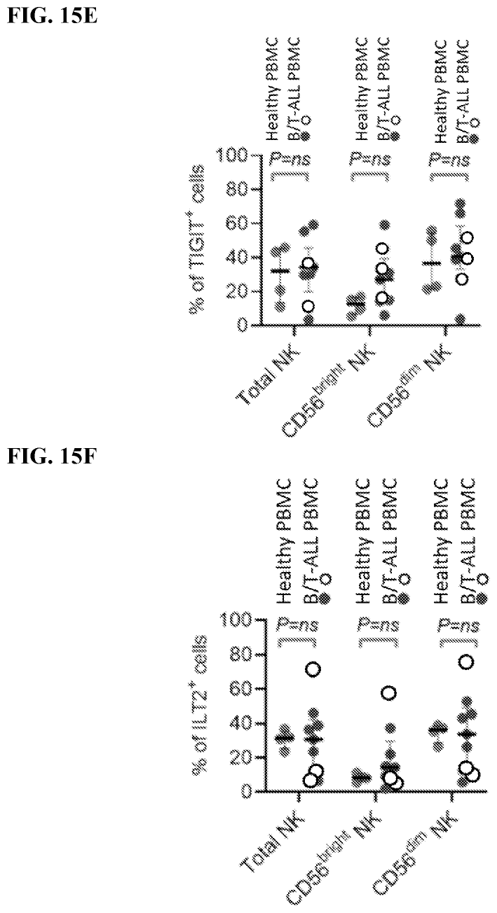

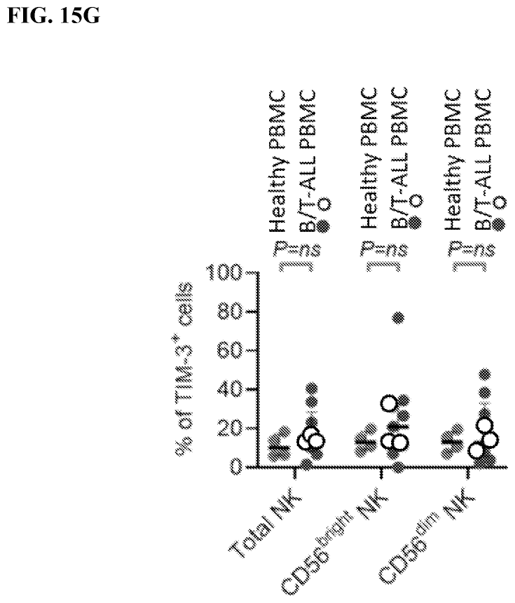

Next, using CyTOF, it was investigated whether chronic activation and impaired cytotoxicity of ALL NK cells are associated with upregulation of checkpoint markers that indicate NK exhaustion, including CTLA-4, PD-1, PD-L1, PD-L2, LAG-3, TIM-3, TIGIT, KLRG1, and ILT2. Expression of immune checkpoints was unaffected in BMMC except for decrease of PD-L1 in the CD56^{bright} subset (FIG. 14). In PBMC, significant increases in LAG-3⁺ total and CD56^{dim} NK cells was observed, and significant increases in KLRG1⁺ and PD-L2⁺ CD56^{bright} NK cells of ALL patients (FIG. 5D). It was inferred that chronic NK activation in ALL could lead to their exhaustion and further impair their functions.

Among other checkpoints, significantly reduced Siglec-7 in total and CD56^{dim} PBMC NK subsets of ALL patients was found (FIG. 5D). Reduced Siglec-7 on NK cells is associated with the loss of NK effector functions. Siglec-7 increases during NK effector maturation, with the highest frequency of Siglec-7⁺NK cells occurring in the most cytotoxic CD11b⁺CD27⁻ NK fraction and the lowest frequency of Siglec-7⁺NK cells in the least cytotoxic and most immature CD11b⁻CD27⁻ subset. Reduced Siglec-7 in PBMC NK cells of ALL patients validates our observations of increased frequencies of CD11b⁻CD27⁻ immature NK and reduced frequencies of mature cytotoxic CD11b⁺CD27⁻ NK cells in ALL (FIG. 3C). Overall, it was demonstrated the accumulation of dysfunctional and exhausted NK cells with an activated phenotype in B/T-ALL.

### High Frequencies of Activated NK Cells Predict Poor Clinical Prognosis in High-Risk B-ALL

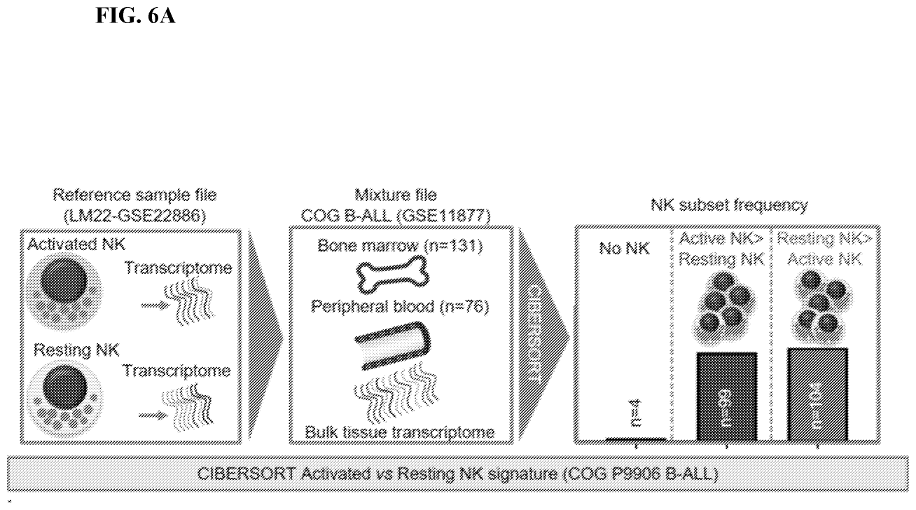

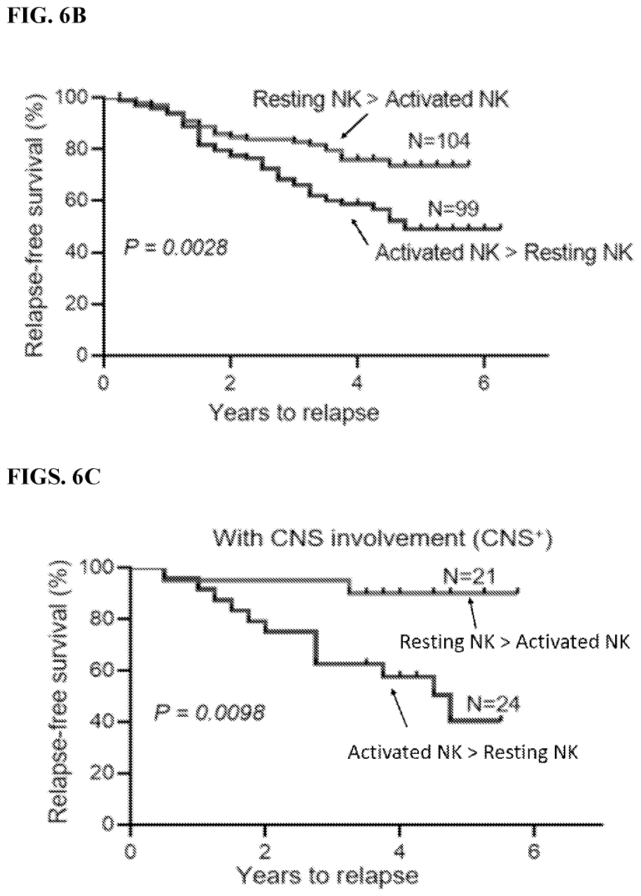

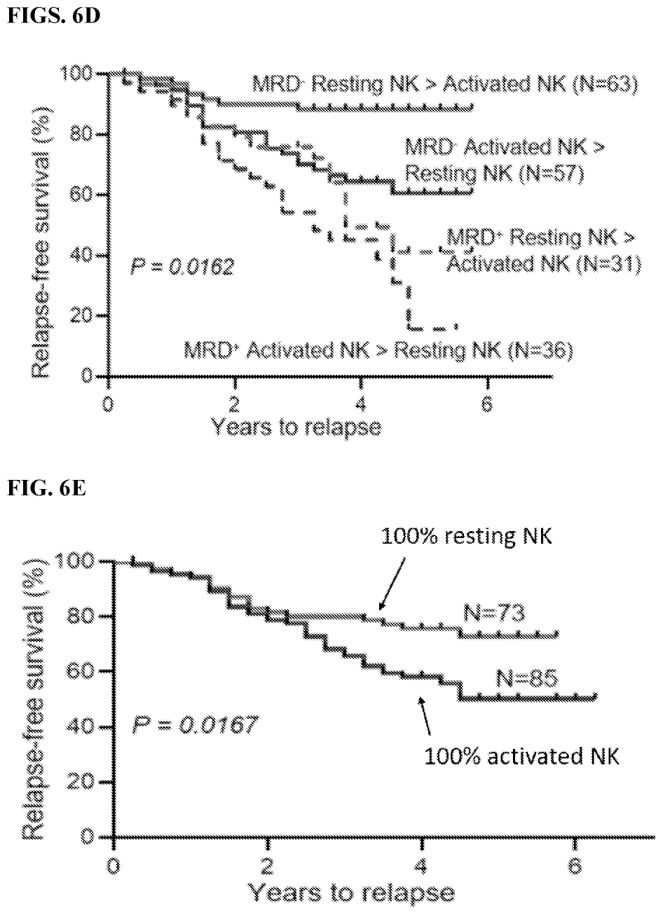

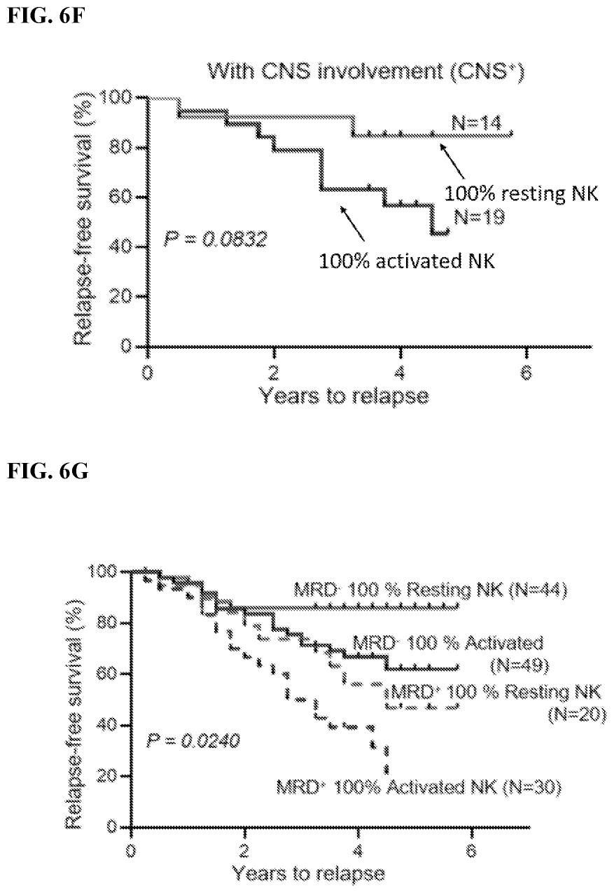

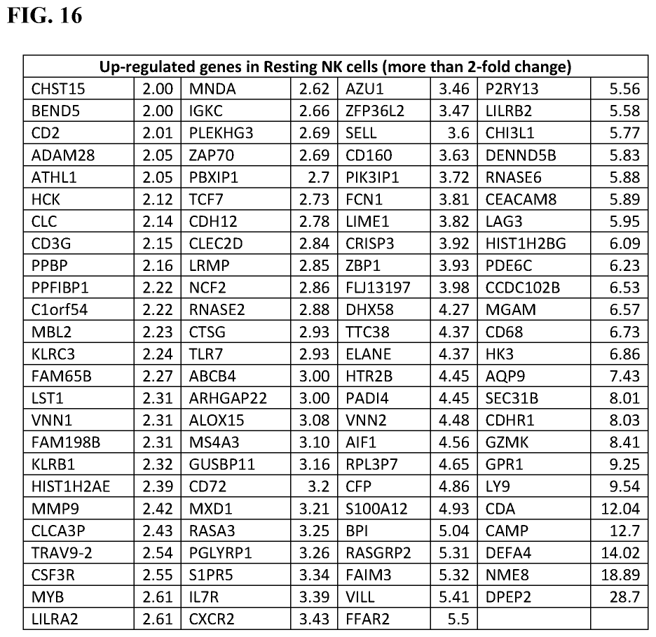

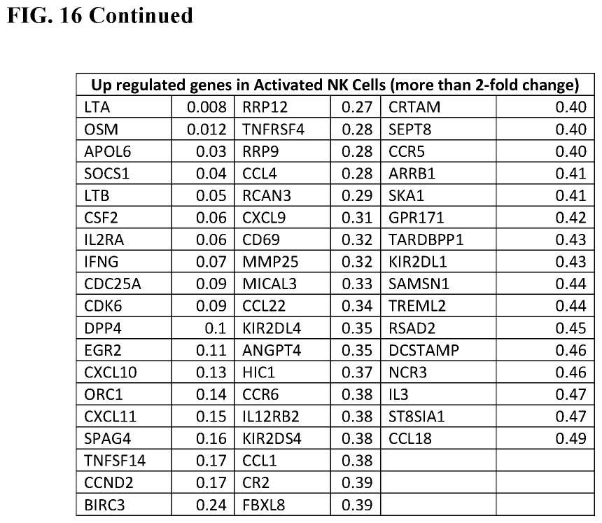

Applicant investigated whether frequencies of activated NK cells predict clinical prognosis in ALL patients. Therefore, using CIBERSORT, Applicant estimated the relative frequencies of activated and resting NK cells in 207 children with B-ALL enrolled in the COG P9906 trial (GSE11877). To estimate activated and resting NK frequencies, the CIBERSORT LM22 reference fil was used, which assigns pan-NK cells into ‘resting NK’ and IL-2/IL-15-‘activated NK’ subsets (FIGS. 6A, 16). It was confirmed that activated and resting NK signatures designated in CIBERSORT include classic NK activation markers and that CIBERSORT is comparable to flow and CyTOF because it uses the expression of lineage markers to distinguish NK cells from other related immune subsets including T cells (FIG. 16).

After excluding 4 B-ALL patients with no detectable NK cells, the remaining patients were assigned to 2 groups based on their relative proportions of resting and activated cells within the total NK fraction as ‘Resting NK>Activated NK’ (n=104) and ‘Activated NK>Resting NK’ (n=99) and compared relapse-free survival (RFS) probabilities between these cohorts. It was observed that patients who had more activated than resting NK cells had shorter RFS (FIG. 6B).

Analyzing poorly prognostic B-ALL with central nervous system involvement (CNS⁺, CNS2 or CNS3), it was found that higher activated NK frequencies significantly shortened RFS (FIG. 6C). Applicant then investigated whether frequencies of activated and resting NK cells can independently predict clinical outcome in patients classified based on minimal residual disease (MRD) as MRD⁺ or MRD⁻ at the end of induction therapy (day 29). Higher relative proportion of activated NK cells predicts significantly worse outcome and shortens RFS in poorly prognostic MRD and better prognostic MRD⁻ patients was observed (FIG. 6D).

Comparing patients with only activated NK and no resting NK (‘100% activated NK’, n=85), against those with no activated and only resting NK (‘100% resting NK’, n=73), patients with 100% activated NK cells had worse prognosis than those with 100% resting NK (FIG. 6E). Finally, Applicant interrogated whether absence of activated or resting NK cells can independently predict clinical outcome in patients classified based on CNS involvement and MRD. It was found that absence of activated NK cells independently predicts favorable clinical outcome in CNS⁺, MRD⁻ and MRD⁺ B-ALL patients (FIGS. 6F-6G). Therefore, enrichment of ‘activated NK’ molecular signature reliably predicts poor clinical outcome in ALL, independently of CNS involvement and MRD status.

### CD56, CD69, Calcium Signaling, and Cytokines are Increased in Poorly Prognostic ALL Patients

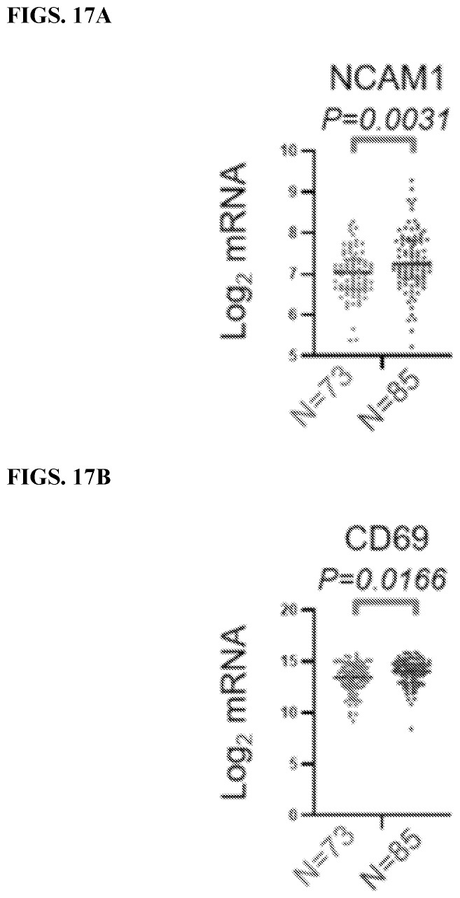

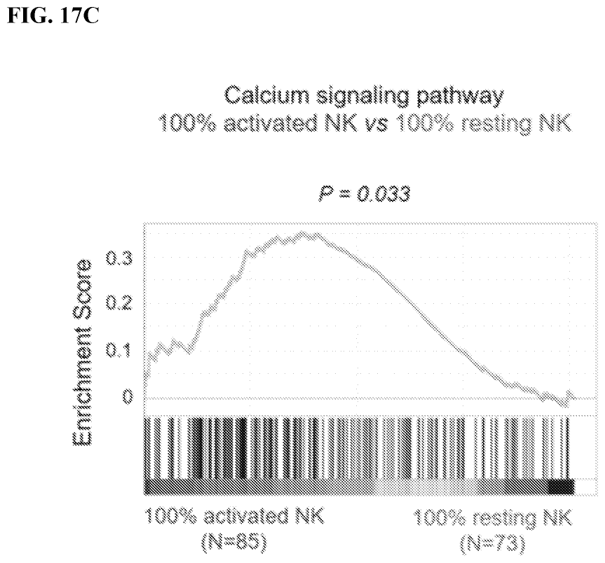

It was confirmed that COG B-ALL patients with only activated NK cells express higher levels of NK activation markers, that was found increased in ALL, compared to their better prognostic 100% resting NK counterparts. First, Applicant showed significantly in patients with 100% activated NK cells than in patients with 100% resting NK cells (FIGS. 17A-17B). Next, using GSEA, Applicant showed that Ca²⁺ signaling is significantly upregulated in the ‘100% activated NK’ group (FIG. 17C). It was predicted that poor prognosis in ALL patients with 100% activated NK cells could stem from the repeated failure of immature chronically activated NK cells to lyse lymphoblasts (FIG. 3-5).

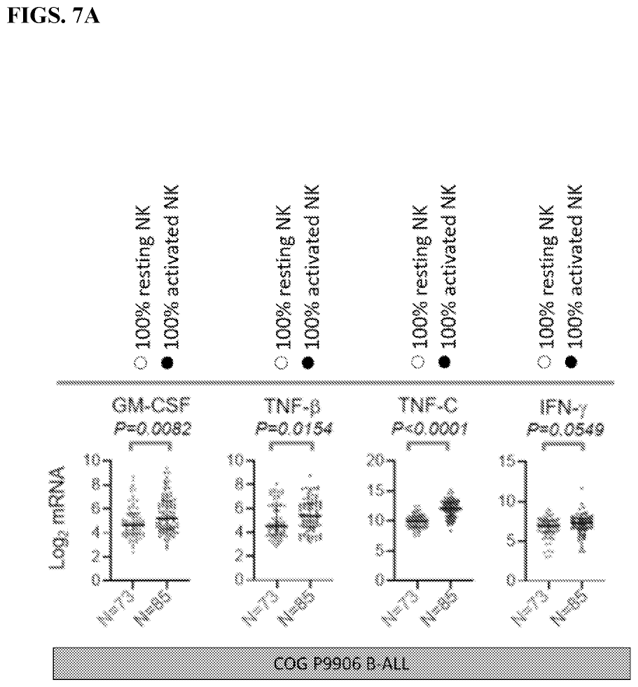

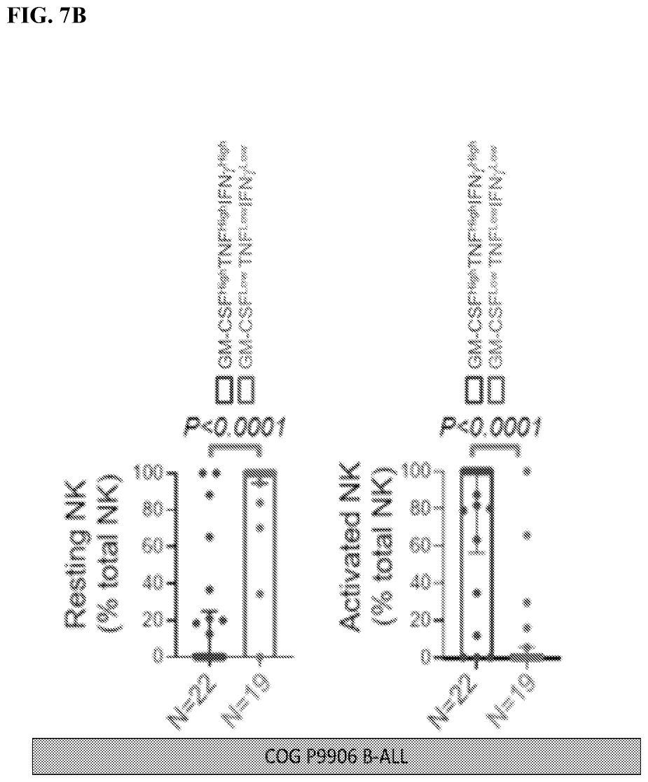

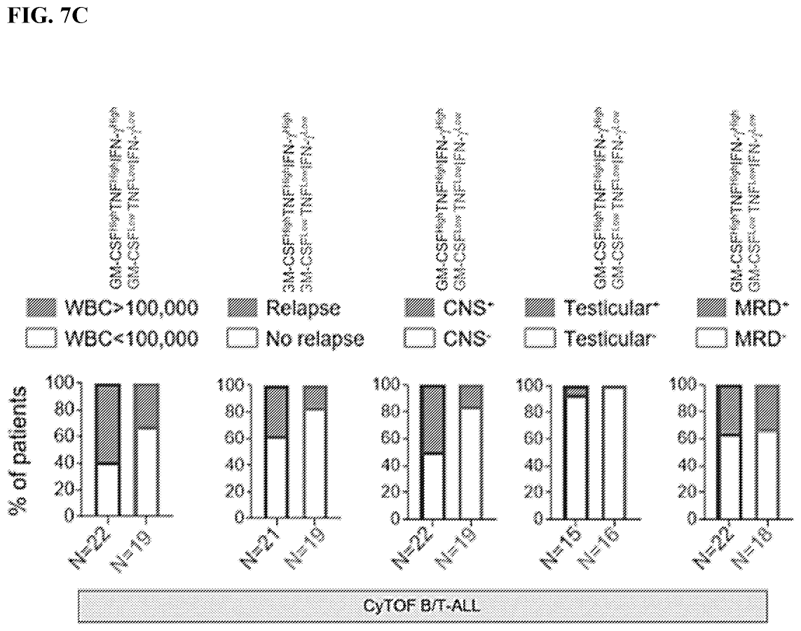

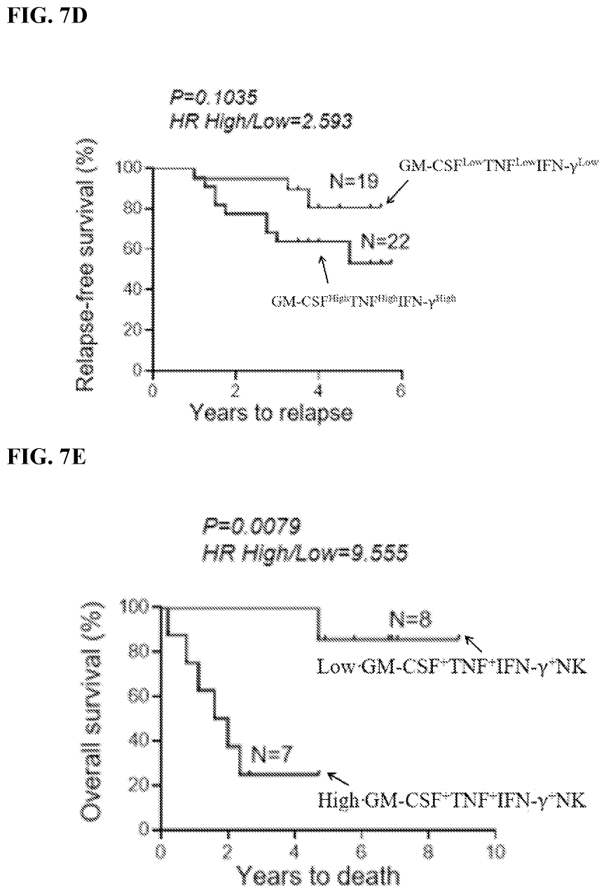

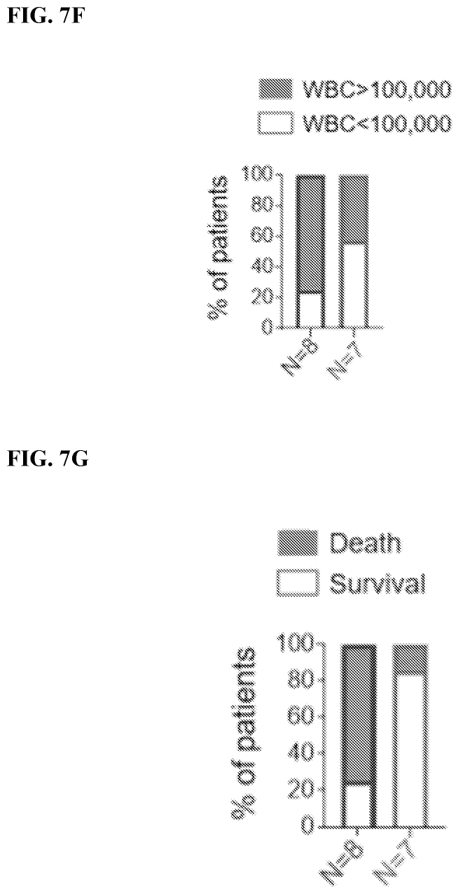

Because ALL NK can produce more cytokines upon stimulation (FIGS. 4A-4F), it was hypothesized that high expression of GM-CSF, TNF, and IFN-γ coincides with the molecular signature of ‘activated NK’ cells in CIBERSORT. To test this: (1) transcript levels of GM-CSF/CSF2, TNF-β/LTA, TNF-C/LTB, and IFN-γ/IFNG in COG B-ALL patients were compared with 100% activated NK or 100% resting NK cells. Since expression of TNF-α was not available in COG, Applicant used TNF-0 and TNF-C, which are also induced in stimulated CD56^{bright} NK cells. Applicant observed significant increases in GM-CSF and TNF mRNA, and a trend towards increased IFN-γ, in 100% activated NK compared to 100% resting NK B-ALL cohort (FIG. 7A). (2) Applicant divided COG B-ALL patients into two groups based on median mRNA expression of GM-CSF, TNF, and IFN-γ, as ‘GM-CSF^{High}TNF^{High}IFN-γ^{High}’ (n=22) and ‘GM-CSF^{Low}INF^{Low}IFN-γ^{Low}’ (n=19), and estimated relative frequencies of activated and resting NK cells in these groups by CIBERSORT. Patients in GM-CSF^{High}TNF^{High}IFN-γ^{High} group had higher frequencies of activated NK cells compared those in GM-CSF^{Low} TNF^{Low} IFN-γ^{Low} group (FIG. 7B). Therefore, the molecular signature of activated NK cells in CIBERSORT coincides with classic NK activation markers, including increased production of cytokines.

### High Frequency of Cytokine-Producing NK Cells Predicts Poor ALL Prognosis

Applicant investigated whether increased cytokine production may at least partly be responsible for the poor clinical outcome of COG B-ALL patients with more activated than resting NK cells. Among markers of NK activation, Applicant chose cytokines to perform clinical correlations because (1) only cytokines were identically regulated in BMMC and PBMC of ALL patients in in vitro studies (FIGS. 4A-4F), and (2) cytokines were differentially regulated between COG B-ALL patients with 100% resting and 100% activated NK cells (FIGS. 7A-7B).

First, Applicant compared proportions of COG B-ALL patients in ‘GM-CSF^{High}TNF^{High}IFN-γ^{High}’ and ‘GM-CSF^{Low} TNF^{Low} IFN-γ^{Low}’ groups with 5 high-risk pediatric ALL features: WBC count at diagnosis>100,000, relapse, CNS⁺, testicular involvement and MRD. Applicant found that ‘GM-CSF^{High}TNF^{High}IFN-γ^{High}’ patients were more likely to have all 5 high-risk features compared to their ‘GM-CSF^{Low}TNF^{Low}IFN-γ^{Low}’ counterparts (FIG. 7C). Furthermore, B-ALL patients in ‘GM-CSF^{High}NF^{High}IFN-γ^{High}’ cohort trended towards shorter RFS as compared to those in ‘GM-CSF^{Low}TNF^{Low}IFN-γ^{Low}’ cohort (FIG. 7D).

Finally, Applicant determined whether high cytokine production specifically by NK cells correlates with poor clinical prognosis. To this end, Applicant interrogated whether high frequencies of GM-CSF⁺ TNF⁺ IFN-γ⁺ NK cells predict poor prognosis in CyTOF B/T-ALL patients (Table 1). Applicant divided ALL patients into ‘High GM-CSF⁺ TNF⁺ IFN-γ⁺ NK’ (n=8) and ‘Low GM-CSF⁺ TNF⁺ IFN-γ⁺ NK’ (n=7) groups based on the median frequencies of stimulated NK cells expressing these proteins. Applicant found that patients in ‘High GM-CSF⁺ TNF⁺ IFN-γ⁺ NK’ group had lower overall survival probability than those in ‘Low GM-CSF⁺ TNF⁺ IFN-γ⁺ NK’ group (FIG. 7E). Applicant also observed that higher proportion of patients in ‘High GM-CSF⁺ TNF⁺ IFN-γ⁺ NK’ group had WBC counts>100,000 and did not survive post induction therapy compared to patients in ‘Low GM-CSF⁺ TNF⁺ IFN-γ⁺ NK’ group (FIGS. 7F-7G). Therefore, accumulation of cytokine-producing NK cells at least partly contributes to increased ALL severity and poor outcome of patients with high activated NK frequencies.

### Discussion

Studies showing that NK cells promote ALL regression in bone marrow transplant recipients underscore the role of NK cells in anti-leukemia immune surveillance. However, the mechanisms by which NK surveillance is perturbed in human ALL, and whether these perturbations in NK surveillance predict clinical outcome in patients were unknown. Applicant addressed these clinically relevant questions.

To delineate how NK surveillance is subverted in human ALL, Applicant immunophenotyped NK cells in B/T-ALL patients using CyTOF and flow cytometry. Applicant found that ALL NK cells are less cytotoxic but exhibit a more activated and immature CD56^{bright}-like signature than their healthy counterparts. Because Applicant observed identical perturbations in NK homeostasis in B- and T-ALL patients, Applicant inferred that identical molecular mechanisms suppress NK surveillance in both ALL lineages.

Applicant found that, despite their increased activation and high cytokine production, ALL NK cells do not lyse NK-sensitive targets as efficiently as those from healthy counterparts. Applicant showed that decreased production of cytotoxic NK effectors in ALL patients may reduce NK cytotoxicity. Reports show that initial failed NK-mediated lysis of target cells leads to continuous engagement of activation receptors on NK cells and promotes Ca²⁺ signaling, ultimately causing NK exhaustion. Therefore, the observations demonstrating increased frequencies of CD94⁺CD69⁺NK cells, reduced frequencies of KIR2DL1⁺NK cells, enhanced Ca²⁺ signaling, and increased NK exhaustion markers in patients prove that dysfunctional hyperactivated NK cells accumulate in ALL. Other reasons for ALL NK dysfunctionality which have not been interrogated here include perturbations in homeostasis of lytic granules.

NK suppression in cancer is caused by resistance of target cancer cells to NK-mediated lysis, and/or defects in NK homeostasis. The findings are clinically relevant: hyperactivated cytokine-producing NK cells predict poor clinical outcome in B/T-ALL patients independent of prognostic factors including MRD and CNS involvement. The results provided herein suggest that activated cytokine-producing NK cells predict poor clinical outcome in both childhood and adult ALL.

The results provided herein studies underscore the therapeutic potential of allogeneic-NK infusions sustaining ALL regression: First, reduced NK frequencies in patients make it challenging to obtain sufficient autologous NK cells for engineering therapies. Second, the inability of NK cells from B/T-ALL patients to lyse NK-sensitive targets may partly explain why autologous hematopoietic transplants fail to induce ALL regression as effectively as allogeneic haploidentical transplants. Therefore, engineering NK therapies from dysfunctional autologous NK cells derived from ALL patients would be a tedious process.

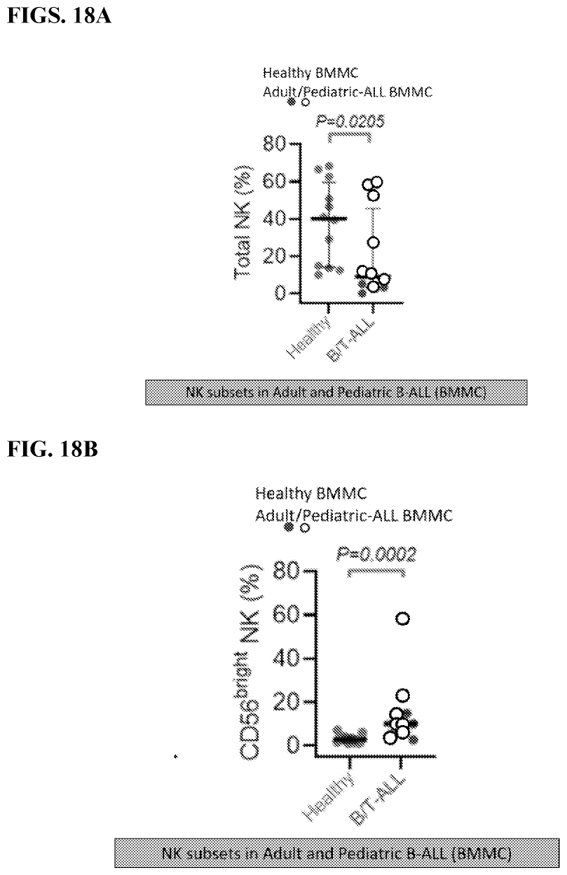

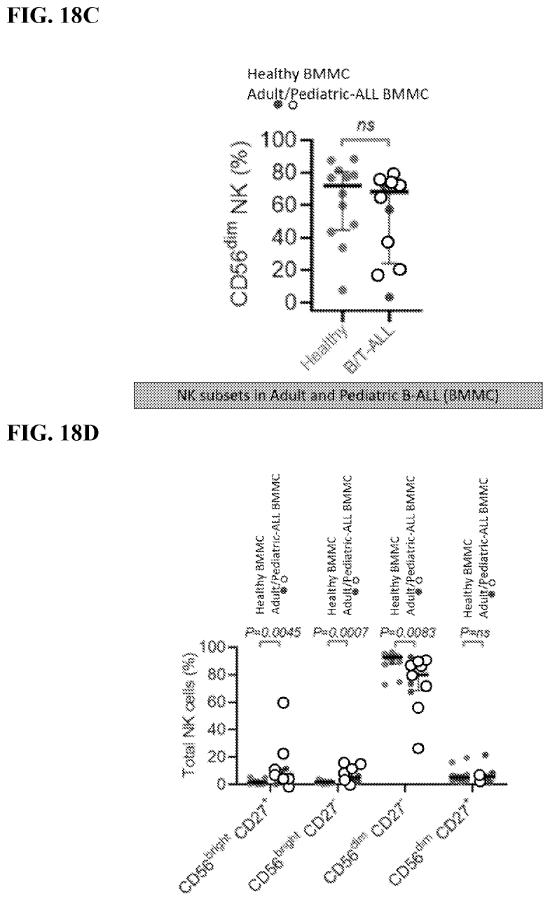

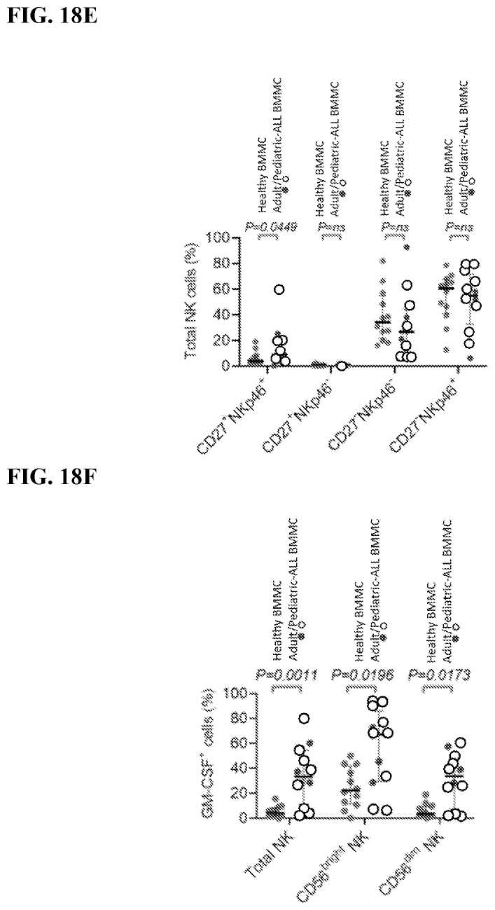

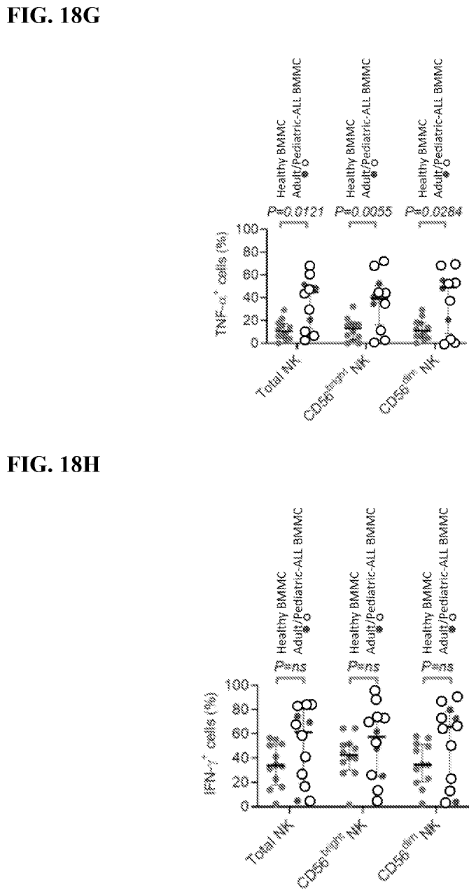

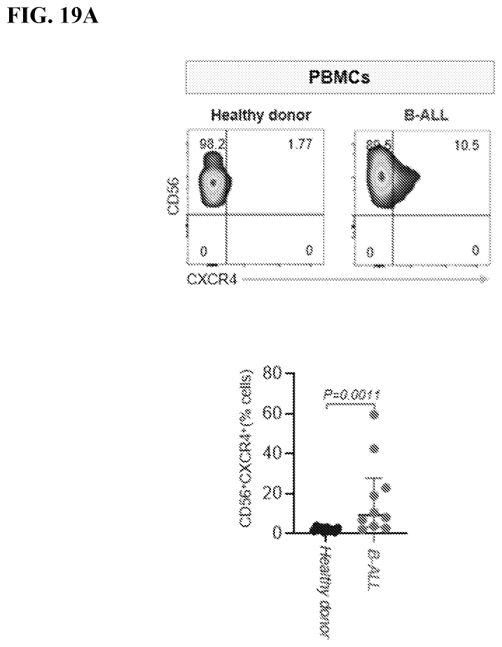

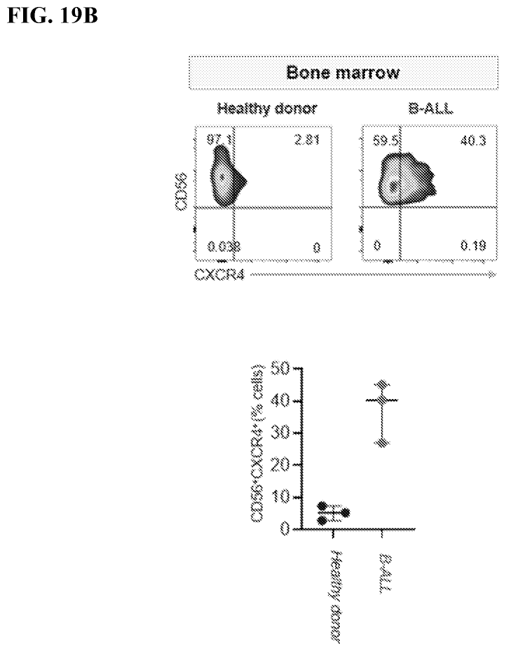

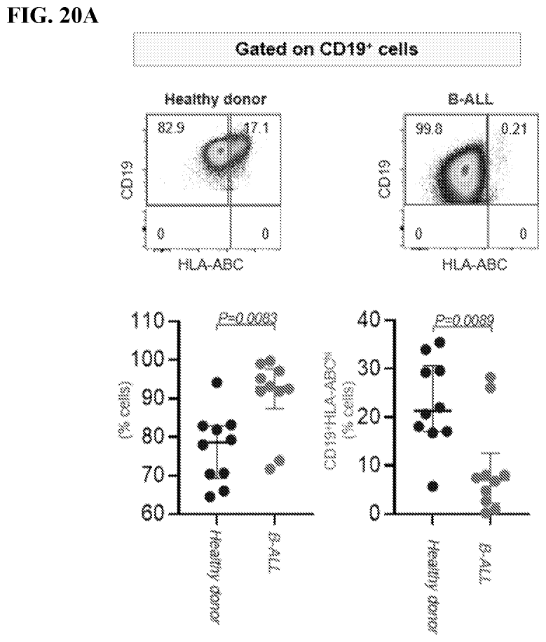

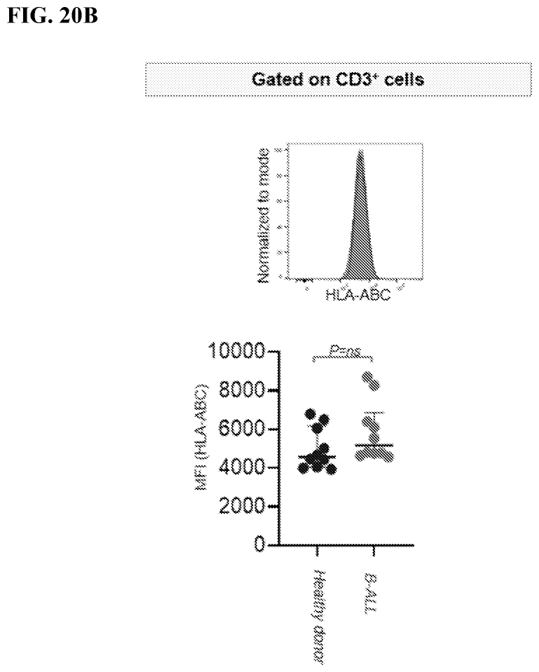

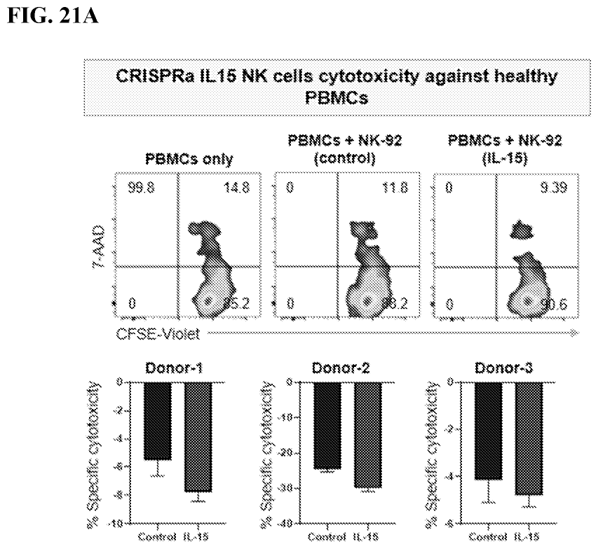

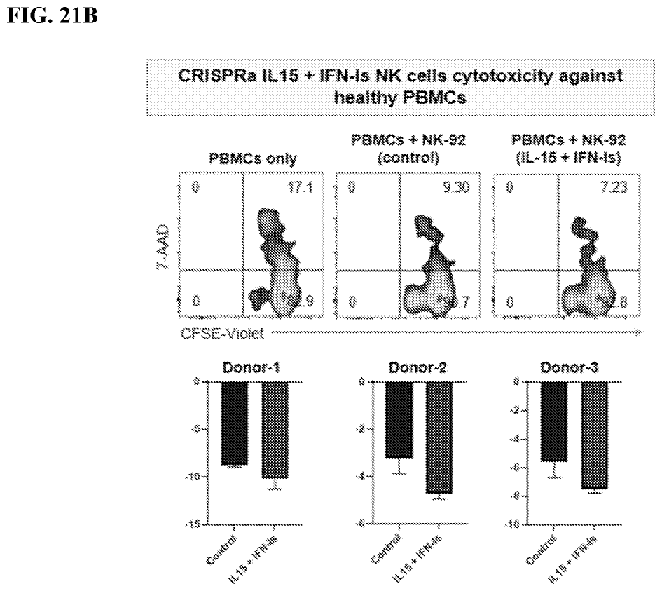

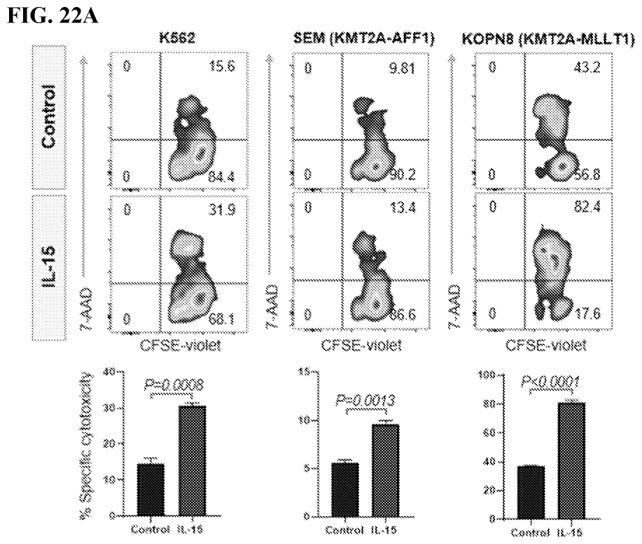

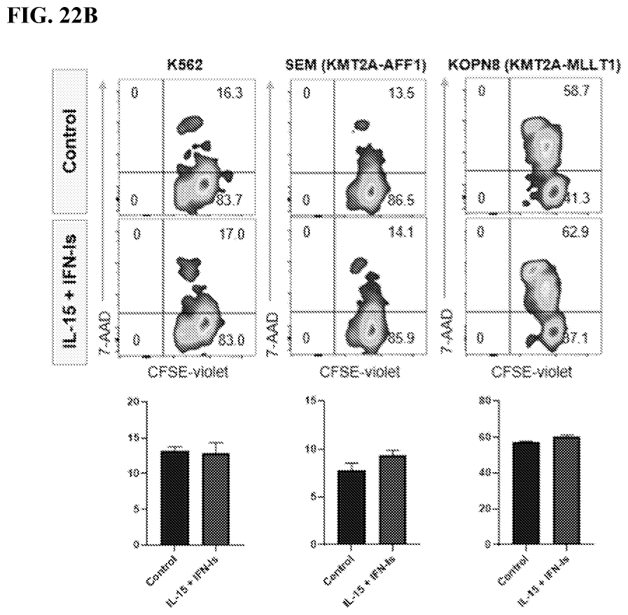

The present results have two limitations: (1) 75% of BMMC B/T-ALL samples used in this study are from pediatric subjects as it is difficult to obtain healthy pediatric bone marrow controls. Despite this apparent pitfall, Applicant found that both pediatric and adult ALL have the same perturbations in NK surveillance compared to healthy donors (FIG. 18). Hence, Applicant conclude that ages of the ALL patients do not determine the presence or absence of NK dysfunction. However, the extent of NK dysfunction in ALL may be associated with age because adult ALL has a worse clinical prognosis than childhood ALL. (2) Reduced NK frequencies in ALL patients and difficulty in manipulating NK cells using viral vectors precluded us from conducting knockdown or rescue experiments showing that modulating CD56^{bright} to CD56^{dim} NK transition restores cytotoxicity of NK cells from ALL patients. Applicant hope to conduct these studies in the future using humanized mice.

Finally, it is important to conduct single cell RNA sequencing to delineate the molecular events leading to the suppression of NK effector maturation in ALL patients, determine whether leukemia inside-out signaling suppresses NK surveillance, and identify the mechanisms of lymphoblast resistance that block NK cytotoxicity in ALL patients.

### REFERENCES

1. Terwilliger T, Abdul-Hay M. Acute lymphoblastic leukemia: a comprehensive review and 2017 update. Blood cancer journal. 2017; 7(6).
2. Bhojwani D, Howard S, Pui C. High-risk childhood acute lymphoblastic leukemia. Clinical lymphoma & myeloma. 2009; 9 Suppl 3(Suppl 3).
3. Bhojwani D, Pui C. Relapsed childhood acute lymphoblastic leukaemia. The Lancet Oncology. 2013; 14(6).
4. Bethesda M. SEER Cancer Stat Facts: Acute Lymphocytic Leukemia; 2020.
5. Paul S, Kantarjian H, Jabbour E. Adult Acute Lymphoblastic Leukemia. Mayo Clinic proceedings. 2016; 91(11).
6. Druker B, Sawyers C, Kantarjian H, et al. Activity of a specific inhibitor of the BCR-ABL tyrosine kinase in the blast crisis of chronic myeloid leukemia and acute lymphoblastic leukemia with the Philadelphia chromosome. The New England journal of medicine. 2001; 344(14).
7. Krivtsov A, Evans K, Gadrey J, et al. A Menin-MLL Inhibitor Induces Specific Chromatin Changes and Eradicates Disease in Models of MLL-Rearranged Leukemia. Cancer cell. 2019; 36(6).
8. Daigle S, Olhava E, Therkelsen C, et al. Potent inhibition of DOT1L as treatment of MLL-fusion leukemia. Blood. 2013; 122(6).
9. Maude S, Frey N, Shaw P, et al. Chimeric antigen receptor T cells for sustained remissions in leukemia. The New England journal of medicine. 2014; 371(16).
10. Lee D, Kochenderfer J, Stetler-Stevenson M, et al. T cells expressing CD19 chimeric antigen receptors for acute lymphoblastic leukaemia in children and young adults: a phase 1 dose-escalation trial. Lancet (London, England). 2015; 385(9967).
11. Brentjens R, Davila M, Riviere I, et al. CD19-targeted T cells rapidly induce molecular remissions in adults with chemotherapy-refractory acute lymphoblastic leukemia. Science translational medicine. 2013; 5(177).
12. Davila M, Riviere I, Wang X, et al. Efficacy and toxicity management of 19-28z CAR T cell therapy in B cell acute lymphoblastic leukemia. Science translational medicine. 2014; 6(224).
13. Liu D, Zhao J, Song Y, Luo X, Yang T. Clinical trial update on bispecific antibodies, antibody-drug conjugates, and antibody-containing regimens for acute lymphoblastic leukemia. Journal of hematology & oncology. 2019; 12(1).
14. Einsele H, Rasche L, Topp M, Martin Kortum K, Duell J. The use of bispecific antibodies to optimize the outcome of patients with acute leukemia, lymphoma and multiple myeloma after SCT. Bone marrow transplantation. 2019; 54(Suppl 2).
15. Salgia R, Kulkarni P. The Genetic/Non-genetic Duality of Drug ‘Resistance’ in Cancer. Trends in cancer. 2018; 4(2).
16. Marine J, Dawson S, Dawson M. Non-genetic mechanisms of therapeutic resistance in cancer. Nature reviews Cancer. 2020.
17. @NCICancerStats. Acute Lymphocytic Leukemia—Cancer Stat Facts: @NCICancerStats; 2021.
18. Witkowski M, Dolgalev I, Evensen N, et al. Extensive Remodeling of the Immune Microenvironment in B Cell Acute Lymphoblastic Leukemia. Cancer cell. 2020; 37(6).
19. Curran E, Godfrey J, Kline J. Mechanisms of Immune Tolerance in Leukemia and Lymphoma. Trends in immunology. 2017; 38(7).
20. Swaminathan S, Hansen A, Heftdal L, et al. MYC functions as a switch for natural killer cell-mediated immune surveillance of lymphoid malignancies. Nature communications. 2020; 11(1).
21. Ortaldo J, Herberman R. Heterogeneity of natural killer cells. Annual review of immunology. 1984; 2.
22. Vivier E, Tomasello E, Baratin M, Walzer T, Ugolini S. Functions of natural killer cells. Nature immunology. 2008; 9(5).
23. Locatelli F, Moretta F, Brescia L, Merli P. Natural killer cells in the treatment of high-risk acute leukaemia. Seminars in immunology. 2014; 26(2).
24. Hu W, Wang G, Huang D, Sui M, Xu Y. Cancer Immunotherapy Based on Natural Killer Cells: Current Progress and New Opportunities. Frontiers in immunology. 2019; 10.
25. Chu J, Deng Y, Benson D, et al. CS1-specific chimeric antigen receptor (CAR)-engineered natural killer cells enhance in vitro and in vivo antitumor activity against human multiple myeloma. Leukemia. 2014; 28(4).
26. Tang X, Yang L, Li Z, et al. First-in-man clinical trial of CAR NK-92 cells: safety test of CD33-CAR NK-92 cells in patients with relapsed and refractory acute myeloid leukemia. American journal of cancer research. 2018; 8(6).
27. Geller M, Miller J. Use of allogeneic NK cells for cancer immunotherapy. Immunotherapy. 2011; 3(12).
28. Veluchamy J, Kok N, van der Vliet H, Verheul H, de Gruijl T, Spanholtz J. The Rise of Allogeneic Natural Killer Cells As a Platform for Cancer Immunotherapy: Recent Innovations and Future Developments. Frontiers in immunology. 2017; 8.
29. Ruggeri L, Capanni M, Urbani E, et al. Effectiveness of donor natural killer cell alloreactivity in mismatched hematopoietic transplants. Science (New York, NY). 2002; 295(5562).
30. Burns L, Weisdorf D, DeFor T, et al. IL-2-based immunotherapy after autologous transplantation for lymphoma and breast cancer induces immune activation and cytokine release: a phase I/II trial. Bone marrow transplantation. 2003; 32(2).
31. Cooley S, Gada P, McKenna D, et al. Successful Haploidentical Hematopoietic Cell Engraftment Using a Non-Myeloablative Preparative Regimen Including Natural Killer (NK) Cells. In: Blood ed. ASH annual meeting. Vol. 112; 2008.
32. Curti A, Ruggeri L, D'Addio A, et al. Successful transfer of alloreactive haploidentical KIR ligand-mismatched natural killer cells after infusion in elderly high risk acute myeloid leukemia patients. Blood. 2011; 118(12).
33. Rubnitz J, Inaba H, Ribeiro R, et al. NKAML: a pilot study to determine the safety and feasibility of haploidentical natural killer cell transplantation in childhood acute myeloid leukemia. Journal of clinical oncology: official journal of the American Society of Clinical Oncology. 2010; 28(6).
34. Fleischer L, Spencer H, Raikar S. Targeting T cell malignancies using CAR-based immunotherapy: challenges and potential solutions. Journal of hematology & oncology. 2019; 12(1).
35. Hartmann J, Schüßller-Lenz M, Bondanza A, Buchholz C. Clinical development of CAR T cells-challenges and opportunities in translating innovative treatment concepts. EMBO molecular medicine. 2017; 9(9).
36. Lin C, Zhang J. Reformation in chimeric antigen receptor based cancer immunotherapy: Redirecting natural killer cell. Biochimica et biophysica acta Reviews on cancer. 2018; 1869(2).
37. Benjamin J, Gill S, Negrin R. Biology and clinical effects of natural killer cells in allogeneic transplantation. Current opinion in oncology. 2010; 22(2).
38. Aversa F, Tabilio A, Velardi A, et al. Treatment of high-risk acute leukemia with T-cell-depleted stem cells from related donors with one fully mismatched HLA haplotype. The New England journal of medicine. 1998; 339(17).
39. Liu E, Tong Y, Dotti G, et al. Cord blood NK cells engineered to express IL-15 and a CD19-targeted CAR show long-term persistence and potent antitumor activity. Leukemia. 2018; 32(2).
40. Rezvani K, Rouce R, Liu E, Shpall E. Engineering Natural Killer Cells for Cancer Immunotherapy. Molecular therapy: the journal of the American Society of Gene Therapy. 2017; 25(8).
41. Rezvani K, Rouce R. The Application of Natural Killer Cell Immunotherapy for the Treatment of Cancer. Frontiers in immunology. 2015; 6.
42. Rouce R, Sekine T, Weber G, et al. Natural Killer Cells In Pediatric Acute Lymphoblastic Leukemia Patients At Diagnosis Demonstrate An Inhibitory Phenotype and Reduced Cytolytic Capacity. In: Blood ed. ASH Annual Meeting. Vol. 122: Blood; 2013.
43. Shah N, Fry T. Mechanisms of resistance to CAR T cell therapy. Nature reviews Clinical oncology. 2019; 16(6).
44. Sotillo E, Barrett D, Black K, et al. Convergence of Acquired Mutations and Alternative Splicing of CD19 Enables Resistance to CART-19 Immunotherapy. Cancer discovery. 2015; 5(12).
45. Gardner R, Wu D, Cherian S, et al. Acquisition of a CD19-negative myeloid phenotype allows immune escape of MLL-rearranged B-ALL from CD19 CAR-T-cell therapy. Blood. 2016; 127(20).
46. Hamieh M, Dobrin A, Cabriolu A, et al. CAR T cell trogocytosis and cooperative killing regulate tumour antigen escape. Nature. 2019; 568(7750).
47. Xie G, Dong H, Liang Y, Ham J, Rizwan R, Chen J. CAR-NK cells: A promising cellular immunotherapy for cancer. EBioMedicine. 2020; 59.
48. Parker K, Migliorini D, Perkey E, et al. Single-Cell Analyses Identify Brain Mural Cells Expressing CD19 as Potential Off-Tumor Targets for CAR-T Immunotherapies. Cell. 2020; 183(1).
49. Bendall S, Simonds E, Qiu P, et al. Single-cell mass cytometry of differential immune and drug responses across a human hematopoietic continuum. Science (New York, NY). 2011; 332(6030).
50. Newman A, Liu C, Green M, et al. Robust enumeration of cell subsets from tissue expression profiles. Nature methods. 2015; 12(5).
51. Tomic A, Tomic I, Dekker C, Maecker H, Davis M. The FluPRINT dataset, a multidimensional analysis of the influenza vaccine imprint on the immune system. Scientific data. 2019; 6(1).
52. Lin D, Gupta S, Maecker H. Intracellular Cytokine Staining on PBMCs Using CyTOF™ Mass Cytometry. Bio-protocol. 2015; 5(1).
53. Kotecha N, Krutzik P, Irish J. Web-based analysis and publication of flow cytometry experiments. Current protocols in cytometry. 2010; Chapter 10.
54. Juelke K, Killig M, Luetke-Eversloh M, et al. CD62L expression identifies a unique subset of polyfunctional CD56dim NK cells. Blood. 2010; 116(8).
55. Menssen A, Edinger G, Grin J, et al. SiPaGene: A new repository for instant online retrieval, sharing and meta-analyses of GeneChip expression data. BMC genomics. 2009; 10.
56. Mootha V K, Lindgren C M, Eriksson K F, et al. PGC-1alpha-responsive genes involved in oxidative phosphorylation are coordinately downregulated in human diabetes. Nat Genet. 2003; 34(3):267-273.
57. Bild A, Febbo PG. Application of a priori established gene sets to discover biologically important differential expression in microarray data. Proc Natl Acad Sci USA. 2005; 102(43):15278-15279.
58. Subramanian A, Tamayo P, Mootha V K, et al. Gene set enrichment analysis: a knowledge-based approach for interpreting genome-wide expression profiles. Proc Natl Acad Sci USA. 2005; 102(43):15545-15550.
59. Freud A, Yu J, Caligiuri M. Human natural killer cell development in secondary lymphoid tissues. Seminars in immunology. 2014; 26(2).
60. Michel T, Poli A, Cuapio A, et al. Human CD56^{bright} NK Cells: An Update. Journal of immunology (Baltimore, Md: 1950). 2016; 196(7).
61. Cooper M, Fehniger T, Turner S, et al. Human natural killer cells: a unique innate immunoregulatory role for the CD56(bright) subset. Blood. 2001; 97(10).
62. Crinier A, Milpied P, Escaliere B, et al. High-Dimensional Single-Cell Analysis Identifies Organ-Specific Signatures and Conserved NK Cell Subsets in Humans and Mice. Immunity. 2018; 49(5).
63. Yang C, Siebert J, Burns R, et al. Heterogeneity of human bone marrow and blood natural killer cells defined by single-cell transcriptome. Nature communications. 2019; 10(1).
64. Kohlmann A, Kipps T, Rassenti L, et al. An international standardization programme towards the application of gene expression profiling in routine leukaemia diagnostics: the Microarray Innovations in LEukemia study prephase. British Journal of Haematology. 2008; 142(5).
65. Kang H, Chen I, Wilson C, et al. Gene expression classifiers for relapse-free survival and minimal residual disease improve risk classification and outcome prediction in pediatric B-precursor acute lymphoblastic leukemia. Blood. 2010; 115(7).
66. Harvey R, Mullighan C, Wang X, et al. Identification of novel cluster groups in pediatric high-risk B-precursor acute lymphoblastic leukemia with gene expression profiling: correlation with genome-wide DNA copy number alterations, clinical characteristics, and outcome. Blood. 2010; 116(23).
67. Harvey R, Mullighan C, Chen I, et al. Rearrangement of CRLF2 is associated with mutation of JAK kinases, alteration of IKZF1, Hispanic/Latino ethnicity, and a poor outcome in pediatric B-progenitor acute lymphoblastic leukemia. Blood. 2010; 115(26).
68. Haferlach T, Kohlmann A, Wieczorek L, et al. Clinical utility of microarray-based gene expression profiling in the diagnosis and subclassification of leukemia: report from the International Microarray Innovations in Leukemia Study Group. Journal of clinical oncology: official journal of the American Society of Clinical Oncology. 2010; 28(15).
69. Silva A, Andrews D, Brooks A, Smyth M, Hayakawa Y. Application of CD27 as a marker for distinguishing human NK cell subsets. International immunology. 2008; 20(4).
70. Lopez-Verges S, Milush J, Pandey S, et al. CD57 defines a functionally distinct population of mature NK cells in the human CD56dimCD16+ NK-cell subset. Blood. 2010; 116(19).
71. Yu J, Mao H, Wei M, et al. CD94 surface density identifies a functional intermediary between the CD56bright and CD56^{dim} human NK-cell subsets. Blood. 2010; 115(2).
72. Fu B, Wang F, Sun R, Ling B, Tian Z, Wei H. CD11b and CD27 reflect distinct population and functional specialization in human natural killer cells. Immunology. 2011; 133(3):350-359.
73. de Andrade L, Smyth M, Martinet L. DNAM-1 control of natural killer cells functions through nectin and nectin-like proteins. Immunology and cell biology. 2014; 92(3).
74. Stannard K, Lemoine S, Waterhouse N, et al. Human peripheral blood DNAM-1 neg NK cells are a terminally differentiated subset with limited effector functions. Blood advances. 2019; 3(11).
75. Wagner J A, Rosario M, Romee R, et al. CD56^{bright} NK cells exhibit potent antitumor responses following IL-15 priming. J Clin Invest. 2017; 127(11):4042-4058.
76. Jacobs R, Hintzen G, Kemper A, et al. CD56^{bright} cells differ in their KIR repertoire and cytotoxic features from CD56^{dim} NK cells. European journal of immunology. 2001; 31(10).
77. Borrego F, Robertson M, Ritz J, Pena J, Solana R. CD69 is a stimulatory receptor for natural killer cell and its cytotoxic effect is blocked by CD94 inhibitory receptor. Immunology. 1999; 97(1).
78. Zhou X, Friedmann K, Lyrmann H, et al. A calcium optimum for cytotoxic T lymphocyte and natural killer cell cytotoxicity. The Journal of physiology. 2018; 596(14).
79. Khan M, Arooj S, Wang H. NK Cell-Based Immune Checkpoint Inhibition. Frontiers in immunology. 2020; 11.
80. Montes-Casado M, Ojeda G, Aragoneses-Fenoll L, et al. ICOS deficiency hampers the homeostasis, development and function of NK cells. PloS one. 2019; 14(7).
81. Merino A, Zhang B, Dougherty P, et al. Chronic stimulation drives human NK cell dysfunction and epigenetic reprograming. The Journal of clinical investigation. 2019; 129(9).
82. Tao L, Wang S, Yang L, Jiang L, Li J, Wang X. Reduced Siglec-7 expression on NK cells predicts NK cell dysfunction in primary hepatocellular carcinoma. Clinical and experimental immunology. 2020; 201(2).
83. Brunetta E, Fogli M, Varchetta S, et al. The decreased expression of Siglec-7 represents an early marker of dysfunctional natural killer-cell subsets associated with high levels of HIV-1 viremia. Blood. 2009; 114(18).
84. Zheng Y, Ma X, Su D, et al. The Roles of Siglec7 and Siglec9 on Natural Killer Cells in Virus Infection and Tumour Progression. Journal of immunology research. 2020; 2020.
85. Shao J, Yin W, Zhang Q, et al. Siglec-7 Defines a Highly Functional Natural Killer Cell Subset and Inhibits Cell-Mediated Activities. Scandinavian journal of immunology. 2016; 84(3).
86. Abel A, Yang C, Thakar M, Malarkannan S. Natural Killer Cells: Development, Maturation, and Clinical Utilization. Frontiers in immunology. 2018; 9.
87. Anft M, Netter P, Urlaub D, Prager I, Schaffner S, Watzl C. NK cell detachment from target cells is regulated by successful cytotoxicity and influences cytokine production. Cellular & molecular immunology. 2020; 17(4).
88. Messai Y, Noman M, Hasmim M, et al. ITPR1 protects renal cancer cells against natural killer cells by inducing autophagy. Cancer research. 2014; 74(23).
89. Messai Y, Noman M, Janji B, Hasmim M, Escudier B, Chouaib S. The autophagy sensor ITPR1 protects renal carcinoma cells from NK-mediated killing. Autophagy. 2015.
90. Vaeth M, Kahlfuss S, Feske S. CRAC Channels and Calcium Signaling in T Cell-Mediated Immunity. Trends in immunology. 2020; 41(10).
91. Krzewski K, Coligan J. Human NK cell lytic granules and regulation of their exocytosis. Frontiers in immunology. 2012; 3.
92. Lehmann C, Zeis M, Schmitz N, Uharek L. Impaired binding of perforin on the surface of tumor cells is a cause of target cell resistance against cytotoxic effector cells. Blood. 2000; 96(2).
93. Parkhurst M R, Riley J P, Dudley M E, Rosenberg S A. Adoptive transfer of autologous natural killer cells leads to high levels of circulating natural killer cells but does not mediate tumor regression. Clin Cancer Res. 2011; 17(19):6287-6297.
94. Geller M A , Cooley S, Judson P L, et al. A phase II study of allogeneic natural killer cell therapy to treat patients with recurrent ovarian and breast cancer. Cytotherapy. 2011; 13(1):98-107.
95. Nguyen A, Khoo W, Moran I, Croucher P, Phan T. Single Cell RNA Sequencing of Rare Immune Cell Populations. Frontiers in immunology. 2018; 9.

1. A method of identifying dysfunctional natural killer (NK) cells in a population of NK cells obtained from a subject having leukemia, wherein the method comprises detecting in said population of NK cells:
(a) an elevated expression level of CD56 relative to a standard control;
(b) an elevated expression level of CD94 relative to the standard control;
(c) an elevated ratio of CD11b⁻CD27⁻ NK cells to CD11b⁺CD27⁻ NK cells relative to the standard control;
(d) the presence of CD69;
(e) the presence of a cytokine; or
(f) calcium (Ca²⁺) mobilization;
thereby identifying the dysfunctional NK cells.

2. The method of claim 1, wherein the cytokine is Interferon gamma (IFN-γ), Tumor necrosis factor (TNF), Granulocyte-macrophage colony-stimulating factor (GM-CSF), Macrophage inflammatory protein 1 beta (MIP-1β), Interleukin-2 (IL-2), or combinations thereof.

3. (canceled)

4. The method of claim 1, further comprising detecting the absence of DNAX Accessory Molecule-1 (DNAM 1), Killer cell immunoglobulin-like receptor 2DL1 (KIR2DL1), CD57, Siglec-7, or combinations thereof in said population of NK cells.

5. The method of claim 1, further comprising detecting the presence of cytotoxic granules in said population of NK cells.

6. (canceled)

7. The method of claim 1, further comprising detecting the presence of one or more activation markers in said population of NK cells.

8. (canceled)

9. The method of claim 1, further comprising detecting the presence of one or more checkpoint markers in said population of NK cells.

10. (canceled)

11. The method of claim 1, further comprising detecting the presence of C-X-C chemokine receptor type 4 (CXCR4) in said population of NK cells.

12. (canceled)

13. The method of claim 1, wherein the leukemia is acute lymphoblastic leukemia (ALL).

14. (canceled)

15. The method of claim 1, wherein the detecting comprises a cytometric method or measuring RNA transcript levels.

16. (canceled)

17. The method of claim 1, wherein 50% or more of the population of NK cells are dysfunctional NK cells.

18. The method of claim 17, further comprising administering to the subject an effective amount of allogeneic NK cells.

19. (canceled)

20. (canceled)

21. A method of treating leukemia in a subject in need thereof, comprising administering to the subject an effective amount of allogeneic NK cells, wherein 50% or more of a population of NK cells obtained from the subject are dysfunctional NK cells.

22. The method of claim 21, comprising obtaining the population of NK cells from the subject and identifying the dysfunctional NK cells prior to administering the effective amount of allogeneic NK cells.

23. The method of claim 22, wherein identifying the dysfunctional NK cells comprising detecting in said population of NK cells:
(a) an elevated expression level of CD56 relative to a standard control;
(b) an elevated expression level of CD94 relative to the standard control;
(c) an elevated ratio of CD11b⁻CD27⁻ cells to CD11b⁺CD27⁻ cells relative to the standard control;
(d) the presence of CD69;
(e) the presence of a cytokine; or
(f) calcium (Ca2+) mobilization.

24.-36. (canceled)

37. The method of claim 21, wherein the subject previously received treatment for leukemia.

38. (canceled)

39. The method of claim 37, wherein the subject has relapsed.

40. (canceled)

41. (canceled)

42. A method of treating leukemia in a subject in need thereof, comprising:
a) identifying dysfunctional natural killer (NK) cells in a population of NK cells obtained from the subject; and
b) administering to the subject an effective amount of allogeneic NK cells;
wherein 50% or more of the population of NK cells are dysfunctional NK cells.

43. The method of claim 42, wherein identifying the dysfunctional natural killer (NK) cells comprises detecting in said population of NK cells:
(a) an elevated expression level of CD56 relative to a standard control;
(b) an elevated expression level of CD94 relative to the standard control;
(c) an elevated ratio of CD11b⁻CD27⁻ NK cells to CD11b⁺CD27⁻ NK cells relative to the standard control;
(d) the presence of CD69;
(e) the presence of a cytokine; or
(f) calcium (Ca2+) mobilization;
thereby identifying the dysfunctional NK cells in the subject.

44.-59. (canceled)

60. A method of determining a probability of survival or relapse in a subject having leukemia, comprising identifying dysfunctional natural killer (NK) cells in a population of NK cells obtained from the subject, wherein 50% or more of the population of NK cells are dysfunctional NK cells indicates that the subject has decreased probability of survival or increased probability of relapse relative to a subject wherein less than 50% of the population of NK cells are dysfunctional NK cells.

61.-74. (canceled)

75. A method of identifying a subject susceptible to leukemia relapse, comprising identifying dysfunctional natural killer (NK) cells in a population of NK cells obtained from the subject, wherein 50% or more of the population of NK cells are dysfunctional NK cells indicates that the subject is susceptible to leukemia relapse.

76.-89. (canceled)
